(19) United States
(12) Patent Application Publication
(10) Pub. No.: US 2014/0134173 A1
(43) Pub. Date: May 15, 2014

(54) COMPOSITION AND METHODS OF USE FOR BINDING MOLECULES TO DICKKOPF-1 OR DICKKOPF-4 OR BOTH

(71) Applicants: Edgar BRAENDLE, Sparta, NJ (US); Patricia RAE, Berkely Heights, NJ (US); Shamina M. RANGWALA, Brookline, MA (US); David Raymond STOVER, Lexington, MA (US); Ann Taylor, North Kingstown, MA (US)

(72) Inventors: Edgar BRAENDLE, Sparta, NJ (US); Patricia RAE, Berkely Heights, NJ (US); Shamina M. RANGWALA, Brookline, MA (US); David Raymond STOVER, Lexington, MA (US); Ann Taylor, North Kingstown, MA (US)

(73) Assignee: NOVARTIS AG, BASEL (CH)

(21) Appl. No.: 13/915,723

(22) Filed: Jun. 12, 2013

Related U.S. Application Data
(63) Continuation of application No. 13/265,900, filed on Oct. 24, 2011.
(60) Provisional application No. 61/176,317, filed on May 7, 2009.

(51) Int. Cl. C07K 16/18; A61K 38/19; A61K 39/395; A61K 45/06

(52) U.S. Cl. CPC C07K 16/18; A61K 45/06; A61K 38/19; A61K 39/39533
USPC 424/139.1

(57) ABSTRACT

Methods of using binding molecules and fragments thereof that bind to the protein target Dickkopf-1 (DKK1), Dickkopf-4 (DKK4) or both (wherein specificity to DKK1 or DKK4 or both is herein denoted as “DKK1/4”) are provided.

### BACKGROUND OF THE INVENTION

The Wnt signaling pathway is involved in the control of embryonic development and neoplastic processes. Extracellular Wnt proteins are responsible for the growth and differentiation of many cell types during embryogenesis, and contribute to the development of many cancers.

There are at least two families of proteins that inhibit Wnt signaling, namely the secreted frizzled-related family and the Dickkopf (DKK) family. The DKK family currently contains four family members, namely DKK1 (human DNA accno. NM_{—}012242; PRT accno. 094907), DKK2 (human accno. NM_{—}014421; PRT accno. NP_{—}055236), DKK3 (human accno. NM_{—}015881; PRT accno. AAQ88744), and DKK4 (human accno. NM_{—}014420; PRT accno. NP_{—}055235).

Dickkopf-1 (DKK1) is a secreted inhibitor of the Wnt/β-catenin signaling pathway. See, e.g., PCT publications WO9922000 to Niehrs; WO9846755 to McCarthy, WO2007/084344 to Shulok et al. DKK1 possesses the ability to inhibit Wnt-induced axis duplication, and genetic analysis indicates that DKK1 acts upstream to inhibit Wnt signaling. DKK1 interacts antagonistically with LRP6, blocking Wnt-mediated signal activation. See e.g., Mao et al. 2001 Nature 411: 321. DKK1 also plays a role in adipogenesis, chondrogenesis, proliferation of the gastrointestinal epithelial proliferation, bone loss associated with rheumatisms, and initiation of hair follicle placode formation. See Online Mendelian Inheritance in Man (“OMIM”) accno. 605189.

Dickkopf-4 (DKK4) is less well characterized but is likewise a secreted inhibitor of the Wnt pathway. DKK4 has been shown to be deposited in plaques in patients with Alzheimer's disease and is expressed in muscle, cerebellum, T-cell, esophagus and lung. See OMIM accno. 605417.

There is a need for compositions and methods to treat cancers, bone density abnormalities, and metabolic disorders, including such agents that interfere or neutralize DKK1 and/or DKK4 mediated antagonism of Wnt signaling.

Wnt proteins play a major role in cell development and are known for regulating adipogenesis. Wnt10b overexpressing ob/ob and agouti mice have significantly less adipose tissue and are more glucose tolerant and insulin sensitive.

### SUMMARY OF THE INVENTION

The present invention relates to compositions and methods of use for binding molecules specific to Dickkopf-1 (“DKK1”), Dickkopf-4 (“DKK4”), or both (wherein specificity to DKK1 or DKK4 or both is herein denoted as “DKK1/4”), for treating DKK1/4-related abnormalities of bone, bone density, metabolism, diabetes, cancers, and the like.

An embodiment of the invention herein provides a binding molecule or an antigen binding portion thereof that selectively binds to and neutralizes a DKK1 and/or a DKK4 polypeptide or a fragment thereof, and its use in treating diseases.

In certain embodiments, the invention provides a method for treating a disorder or condition associated with DKK1 and/or DKK4 (DKK1/4) expression. DKK1- or DKK4-associated diseases include, but are not limited to, myeloma (including multiple myeloma, monoclonal gammopathy of unknown significance (MGUS) or benign monoclonal gammopathy, plateau and smoldering myeloma), malignant fibrous histiocytosis (MFH) (also known as high grade undifferentiated pleomorphic sarcoma), neuroblastoma, beta thalassemia, inflammatory bowel disease, and bone disorders. Further diseases or disorders include, but are not limited to, e.g., bone disorders, including, but not limited to, bone fracture healing, osteolytic lesions—especially osteolytic lesions and metastases associated with a myeloma (especially a multiple myeloma, MGUS, plateau and smoldering myeloma), or with cancers of the bone, breast, colon, melanocytes, hepatocytes, epithelium, esophagus, brain, lung, prostate or pancreas or metastasis thereof; bone loss associated with transplantation; osteopenia, osteoporosis, bone density abnormality, osteosarcoma, and osteolysis. Further diseases or disorders include, but are not limited to, e.g., cancer, various muscle and metabolic diseases, Alzheimer's disease, rheumatism, colitis and/or unwanted hair loss. Also included are disorders of adipogenesis, chondrogenesis, and pigmentation. Additional disorders include, but are not limited to, cardiovascular diseases, e.g., coronary artery disease, vascular calcification, claudication, atherosclerosis, arteriosclerosis, acute heart failure, congestive heart failure, cardiomyopathy, myocardial infarction, angina pectoris, hypertension, hypotension, stroke, ischemia, ischemic reperfusion injury, aneurysm, restenosis, and vascular stenosis. DKK1 and Wnt pathway genes are known to have altered expression in many of these diseases, including MFH (also known as high grade undifferentiated pleomorphic sarcoma) (Matushanasky et al. 2007 J. Clin. Invest. 117: 3248-3257); inflammatory bowel disease (You et al. 2008 Dig. Dis. Sci. 53:1013-1019); osteosarcoma (Lee et al. 2007 Brit. J. Cancer 97: 1552-1559; Gregory et al. 2003 J. Biol. Chem. 278:28067-28078); bone marrow (skeletal) metastases (Granchi et al. 2008 Int. J. Cancer 123:1526-1535); and lung cancer and esophageal squamous cell carcinomas (ESCC) (Yamabuki et al. 2007 Cancer Res. 67:2517-2525). Specific muscle and metabolic diseases associated with DKK1 or DKK4 include: insulin resistance, non-insulin-dependent diabetes mellitus (NIDDM), hypoinsulinemia, diabetes (especially type 2 diabetes mellitus, or glucocorticoid or other drug associated diabetes), obesity, weight loss, weight loss maintenance, anorexia nervosa, bulimia, cachexia, syndrome X, metabolic syndrome, post-prandial hyperglycemia, post prandial hyperlipidemia and/or hypertriglyceridemia, hypoglycemia, hyperglycemia, hyperuricemia, hyperinsulinemia, hypercholesterolemia, hyperlipidemia, dyslipidemia, mixed dyslipidemia, hypertriglyceridemia, pancreatitis, nonalcoholic fatty liver disease, and muscle trauma, atrophy, wasting, degeneration, repair, regeneration. In a related embodiment, the cancer to be treated is a myeloma, such as MGUS, multiple myeloma or smoldering or plateau myeloma, a cancer of the bone, breast, colon, melanocytes, hepatocytes (e.g., hepatocellular carcinoma (HCC)), epithelium, esophagus, brain, lung, prostate or pancreas or metastasis thereof.

The method involves administering to a subject in need thereof an effective amount of a pharmaceutical composition comprising a binding molecule of the invention.

A neutralizing DKK1/4 binding molecule of the invention is suitable for treating human patients having, or at risk for, a cholesterol-related disorder, including, but not limited to, elevated cholesterol or a condition associated with elevated cholesterol, e.g., a lipid disorder (e.g., hyperlipidemia, type I, type II, type III, type IV, or type V hyperlipidemia, secondary hypertriglyceridemia, hypercholesterolemia, xanthomatosis, cholesterol acetyltransferase deficiency). DKK1/4 binding molecules are also suitable for treating human patients having cardiovascular disease, and patients at risk for this disease, e.g., due to the presence of one or more risk factors (e.g., hypertension, cigarette smoking, diabetes, obesity, or hyperhomocysteinemia).

In certain embodiments, any of the above methods further involve administering a chemotherapeutic or other pharmaceutically active agent. In a related embodiment, the chemotherapeutic agent is an anti-cancer agent. In another related embodiment, the chemotherapeutic agent is an anti-osteoporotic agent. In one embodiment the binding molecule is administered in combination with one or more bone anabolic, weight loss therapy and/or diabetes therapy.

In one embodiment, the binding molecule is a DKK1/4 neutralizing binding molecule (i.e., it specifically neutralizes DKK1 or DKK4 or both). In various embodiments, the antigen-binding portion of the DKK1/4 neutralizing binding molecule does not bind a DKK2 or a DKK3.

In one embodiment the binding molecule or an antigen binding portion thereof is arranged within an immunoglobulin-like scaffold, such as a framework selected from, e.g., a human, humanized, humaneered, shark or camelid scaffold, and/or may additionally be recombinant, chimeric, or CDR grafted antibodies. For instance, technology designed to minimize the Human Anti-murine Antibody response (humaneering technology of Kalobios or humanization technology of PDL) are contemplated within the invention. Further, antigen binding portions specific to DKK1 or DKK4 may be within non-immunoglobulin-like scaffold, including, e.g., arrayed within an adnectin, fibrinogen, ankyrin-derived repeats, etc. type of framework.

In one embodiment, the DKK1 binding molecule is characterized as having an antigen-binding region that is specific for target protein DKK1, and the binding molecule or functional fragment binds to DKK1 or a fragment thereof. In a related embodiment, the DKK4 binding molecule is characterized as having an antigen-binding region that is specific for target protein DKK4, and the binding molecule or functional fragment binds to DKK4 or a fragment thereof. In one embodiment, the binding molecule or antigen-binding portion thereof binds to a DKK1 or a DKK4 polypeptide or both, but not to a DKK2 or DKK3 polypeptide.

In another embodiment the binding molecule or an antigen binding portion thereof is monoclonal. In another embodiment, the antigen-binding portion is polyclonal. In various embodiments, the DKK1 binding molecule or an antigen binding portion thereof binds a peptide consisting of 30 contiguous amino acids of a DKK1 or a DKK4 polypeptide. In one embodiment the binding molecule of the invention binds a DKK1 or DKK4 epitope comprising non-contiguous amino acids.

In a related embodiment, the binding to DKK1 or DKK4 is determined at least by one of the following assays: inhibition of DKK1 or DKK4 antagonism of Wnt-signaled transcription; surface plasmon resonance affinity determination, enzyme-linked immunosorbent assay binding; electrochemiluminescence-based binding analysis; FMAT, SET, SPR, ALP, TopFlash, blood serum concentration of biomarkers such as osteocalcin (OCN), procollagen type 1 nitrogenous propeptide (P1NP) and osteoprotegrin (OPG), and binding to cell surface receptor(s) such as Frizzled (Fz), LRP (LRP5/6) or Kremen (Krm). In certain embodiments, the Dkk1 binding molecule or antigen-binding portion possesses at least one of the following properties: selectivity for DKK1 that is at least 10³-fold, 10⁴-fold or 10⁵-fold greater than for human DKK2 or DKK3; binds to DKK1 or DKK4 with a Kₒₙ of less than 100 nM, 50 nM, 10 nM, 1.0 nM, 500 pM, 100 pM, 50 pM or 10 pM; and has an off-rate for DKK1 of less than 10⁻² per sec, 10⁻³ per sec, 10⁻⁴ per sec, or 10⁻⁵ per sec.

In a related embodiment, a binding molecule of the invention competes with DKK1 and/or DKK4 for binding to LRP5/6. In a related embodiment, a binding molecule of the invention competes with DKK1 and/or DKK4 for binding to Krm.

In another embodiment, the invention provides an isolated antigen-binding region of any of the above binding molecules or functional fragments thereof, and amino acid sequences of these. Thus in certain embodiments, the invention provides isolated amino acid sequences selected from the group of SEQ ID NOs: 2-20 and SEQ ID NOs: 40-72 and conservative or humaneered variants of these sequences.

In another embodiment, the invention provides nucleotide sequences and polypeptide sequences for the binding molecules of the invention, including especially those for DKK1/4 antibodies, the CDR1, CDR2, CDR3 regions of the heavy and light chains, as well as for the various framework regions and the scaffolds.

In one embodiment, sequences are optimized for expression, for production and clinical use. Characteristics to be optimized for clinical use include but are not limited to, e.g., half-life, pharmacokinetics (PK), antigenicity, effector function, FcRn clearance, and patient response including antibody dependent cell cytotoxicity (ADCC) or complement dependent cytotoxicity (CDC) activities.

In other embodiments, the invention provides an amino acid sequence having at least 60, 70, 80, 90, 95, 96, 97, 98 or 99% identity with any one or more of the shaded CDR regions (SEQ ID NOs: 49-98) depicted in Table 18, wherein Table 18 provides the heavy variable regions (SEQ ID NOs: 2-20) and light chain variable regions (SEQ ID NOs: 21-39) of the inventive antibodies. In one embodiment, the invention provides an amino acid sequence having at least 60, 70, 80, 90, 95, 96, 97, 98 or 99% identity with a CDR consensus sequence of a V_{H} chain subgroup as provided in any one or more of SEQ ID NOs: 40-48, and/or with a CDR consensus sequence of a V_{L} chain subgroup as provided in any one or more of SEQ ID NOs: 113-118. The cloning scaffold sequences from Table 18 are as shown in SEQ ID NOs: 125-130.

Table 18 provides the heavy chain and light chain variable regions of SEQ ID NOs: 2-39. Sequence for optimized LC and HC variants of the inventive antibodies are provided as SEQ ID NOs: 99, 101, 103, 105, 107, 109 and 111 for DNA, and as SEQ ID NOs: 100, 102, 104, 106, 108, 110 and 112 for the encoded polypeptide, respectively. In one embodiment, the invention provides an amino acid sequence having at least 60, 70, 80, 90, 95, 96, 97, 98 or 99% identity with any one or more of the sequences depicted in SEQ ID NOs: 2-39 and 100, 102, 104, 106, 108, 110 and 112. In one embodiment, the invention provides a nucleotide sequence having at least 60, 70, 80, 90, 95, 96, 97, 98 or 99% identity with any one or more of the sequences depicted in SEQ ID NOs: 99, 101, 103, 105, 107, 109 and 111.

The sequences for the optimized V_{L} chain, more specifically its DNA sense strand, its corresponding antisense strand and its encoded polypeptide, are provided as SEQ ID NOs: 119-121, respectively. The sequences for the optimized V_{H} chain, more specifically its DNA sense strand, its corresponding antisense strand and its encoded polypeptide, are provided as SEQ ID NOs: 122-124, respectively. In one embodiment, the invention provides an amino acid sequence having at least 60, 70, 80, 90, 95, 96, 97, 98 or 99% identity with a sequence depicted in SEQ ID NOs:121 or 124. In one related embodiment, the invention provides, a nucleotide sequence having at least 60, 70, 80, 90, 95, 96, 97, 98 or 99% identity with a sequence depicted in SEQ ID NOs: 119-120 and 122-123.

In a certain embodiment, any of the above isolated antibodies is an IgG. In a related embodiment, any of the above isolated antibodies is an IgG1, an IgG2, an IgG3 or an IgG4. In another embodiment, the antibody is an IgE, an IgM, an IgD or an IgA. In a related embodiment, the invention is selected from a monoclonal or a polyclonal antibody composition. In further embodiments, the antibody is chimeric, humanized, humaneered, recombinant, etc.

Functional fragments include Fv and Fab fragments (including single chain versions such as scFv), as well other antigen-binding regions of an antibody of the invention, including those that are linked to a non-immunoglobulin scaffold and heavy chain antibodies such as camelid and shark antibodies and nanobodies. In a related embodiment, the isolated antibody as described above is an IgG. In another related embodiment, the isolated antibody as described above is an IgG1, an IgG2, IgG3 or an IgG4. In another embodiment, the antibody is an IgE, an IgM or an IgA. In a related embodiment, the invention is a polyclonal antibody composition.

In one embodiment, the invention provides an isolated human or humanized binding molecule or functional fragment of it, having an antigen-binding region that is specific for an epitope of DKK1, and the binding molecule or functional fragment binds to DKK1 or DKK4, or otherwise blocks binding of DKK1 or DKK4 to a cell surface receptor (e.g., receptors such as LRP5/6, Kremen, Frizzled). In certain embodiments the binding molecule or fragment of it prevents, treats, or ameliorates development of osteolytic lesions. In other embodiments, the anti-DKK composition of the invention prevents, treats, or ameliorates a DKK1- or DKK4-associated cancer or disease.

In one embodiment, the invention provides an isolated human or humanized binding molecule or functional fragment of it, having an antigen-binding region that is specific for an epitope of target DKK1 or DKK4, and the epitope contains six or more amino acid residues from a polypeptide fragment comprising the CYS1-linker-CYS2 domains of DKK1 and/or DKK4. In a related embodiment, the epitope is a conformational epitope. In one embodiment, the epitope resides within the CYS2 domain. In a particular embodiment, the epitope comprises a modified amino acid residue. In a related embodiment, the epitope contains at least one glycosylated amino acid residue.

In another embodiment, the invention provides a pharmaceutical composition having at least one of any or more of the above binding molecules or functional fragments or conservative variants, and a pharmaceutically acceptable carrier or excipient of it.

In another embodiment, any of the above human or humanized binding molecules or fragments thereof are synthetic.

In another embodiment, the invention provides a pharmaceutical composition of any of the above binding molecules or functional fragments thereof and an additional therapeutic agent. The additional therapeutic agent can be selected from the group consisting of an anti-cancer agent; an anti-osteoporotic agent; an antibiotic; an antimetabolic agent; an antidiabetic agent; an anti-inflammatory agent; an anti-angiogenic agent; a growth factor; a bone anabolic, a weight loss therapy, an antidiabetic agent, a hypylipidemic agent, and anti-obesity agent, an anti-hypertensive agent, and/or an agonist of peroxisome proliferators-activator receptors (PPARs) and a cytokine.

The invention further relates to a method of preventing or treating a DKK1-, DKK4- or DKK1/4-associated disease or disorder in a mammal, particularly a human, with a combination of pharmaceutical agents that comprises:

(a) a DKK1/4 binding molecule of the invention; and

(b) one or more pharmaceutically active agents; and optionally

(c) a pharmaceutically acceptable carrier;

wherein at least one pharmaceutically active agent is an anti-cancer therapeutic.

The invention further relates to pharmaceutical compositions comprising:

(a) a DKK1/4 neutralizing agent; and

(b) a pharmaceutically active agent; and optionally

(c) a pharmaceutically acceptable carrier;

wherein at least one pharmaceutically active agent is a bone anabolic, a weight loss therapeutic or a diabetes therapeutic.

The present invention further relates to a commercial package or product comprising:

(a) a pharmaceutical formulation of a DKK1/4 neutralizing binding molecule; and

(b) a pharmaceutical formulation of a pharmaceutically active agent for simultaneous, concurrent, separate or sequential use;

wherein at least one pharmaceutically active agent is an anti-cancer therapeutic, a bone anabolic, a weight loss therapeutic or a diabetes therapeutic.

### BRIEF DESCRIPTION OF THE FIGURES

FIG. 1 shows that Anti-DKK1/4 antibody has high affinity for human DKK1 (2 pM) with binding kinetics typical for an antibody of this affinity.

FIG. 2A shows a schematic representation of full-length and truncated DKK1.

FIG. 2B depicts binding of a neutralizing anti-DKK1/4 antibody and DKK1 proteins.

FIG. 3 shows that anti-DKK1/4 antibody competitively inhibits DKK1 binding to LRP6.

FIG. 4 shows that Anti-DKK1/4 antibody reactivates DKK1 suppressed Wnt signaling with an apparent EC50 of 0.16 nM.

FIG. 5 shows an in vitro assay established to measure Wnt-mediated osteoblast differentiation of the pluripotent mouse cell line C3H10T1/2 (10T1/2).

FIG. 6 shows the effects of 3 doses of anti-DKK1/4 antibody on tumor growth.

FIG. 7 shows the percent calcified bone in animals treated with PBS, IgG, and anti-DKK1/4.

FIG. 8 shows that anti-DKK1/4 antibody demonstrates equivalent anti-osteolytic activity as Zometa.

FIG. 9 shows that an anti-DKK1/4 antibody's anabolic bone efficacy is dose dependent with minimal efficacious dose between 20 and 60 μg/mouse 3×/week.

FIG. 10A and FIG. 10B show the effect of Wnt1 and DKK1 on RNA expression of differentiation markers where GLUT4 protein expression is increased with Wnt3a and DKK1.

FIG. 11 is a graphic representation of expression levels of differentiation markers PPARγ, C/EBP2 and AP2 from cells treated with Wnt3a, DKK1 and MOR4910 (“BHQ880”).

FIG. 12 depicts GLUT4 levels analyzed with Western blotting.

### DETAILED DESCRIPTION OF THE INVENTION

The present invention relates to uses of isolated DKK1/4 binding molecules, particularly human antibodies, that bind specifically to DKK1 or DKK4 and that inhibit functional properties of DKK1 or DKK4. In one embodiment, the DKK1/4 binding molecule (a molecule binding to DKK1 and/or DKK4) does not specifically bind to DKK2 or DKK3.

As used herein a “DKK1-associated disease or disorder” or a “DKK4-associated disease or disorder” or alternatively a “DKK1/4-associated disease or disorder” (a disease or disorder associated with DKK1 and/or DKK4) includes, but is not limited to, myeloma (including multiple myeloma, MGUS, plateau and smoldering myeloma), malignant fibrous histiocytosis or histiocytoma (MFH), neuroblastoma, beta thalassemia, irritable bowel syndrome, inflammatory bowel disease, and bone disorders. As used herein, reference to DKK1 or DKK4 or both is denoted as “DKK1/4” Further such diseases or disorders include but are not limited to, e.g., bone disorders, including, but not limited to, bone fracture healing, osteolytic lesions and metastases; bone loss associated with transplantation; osteopenia, osteroporosis, bone density abnormality, osteosarcoma, and osteolysis. Further such diseases or disorders include but are not limited to, e.g., cancer, various muscle and metabolic diseases, Alzheimer's disease, rheumatism, colitis and/or unwanted hair loss. Also included are disorders of adipogenesis, chondrogenesis, and skin pigmentation. Additional diseases or disorders include, but are not limited to, cardiovascular disease. In a related embodiment, the cancer to be treated is a myeloma (such as multiple myeloma, MGUS, plateau and smoldering myeloma), or a cancer of the bone, breast, colon, melanocytes, hepatocytes, epithelium, esophagus, brain, lung, prostate or pancreas or metastasis thereof. A subject may likewise have a DKK1/4 associated disease or disorder if the subject has, or is at risk for, elevated cholesterol or a condition associated with elevated cholesterol, e.g., a lipid disorder (e.g., hyperlipidemia, type I, type II, type III, type IV, or type V hyperlipidemia, secondary hypertriglyceridemia, hypercholesterolemia, xanthomatosis, cholesterol acetyltransferase deficiency), or if the subject has a cardiovascular disease, or is at risk for this disorder, e.g., due to the presence of one or more risk factors (e.g., hypertension, cigarette smoking, diabetes, obesity, or hyperhomocysteinemia). Data presented in this application show that that treatment of 3T3L1 fibroblasts with DKK1 antibody MOR4910 inhibits differentiation into adipocytes. The inhibition of adipocytes can be applied to metabolic diseases and conditions related to the activity of adipocytes and body fat such as obesity, weight loss maintenance and hyperlipidemia, and the reduction of body fat in cancer patients

The method involves administering to a subject in need thereof an effective amount of a pharmaceutical composition comprising a binding molecule of the invention.

In certain embodiments, the binding molecules of the invention are antibodies derived from particular heavy and light chain sequences and/or comprise particular structural features such as CDR regions comprising particular amino acid sequences. The invention provides isolated antibodies, methods of making such antibodies, immunoconjugates and bispecific molecules comprising such antibodies and pharmaceutical compositions containing the antibodies, immunoconjugates or bispecific molecules of the invention. The invention also relates to methods of using the antibodies to inhibit a disorder or condition associated DKK1 or DKK4, or both, as provided herein.

In order that the present invention may be more readily understood, certain terms are first defined. Additional definitions are set forth throughout the detailed description.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and capable of the transmission of such a signal across the plasma membrane of a cell. An example of a “cell surface receptor” of the present invention is a receptor to which the DKK1 or the DKK4 protein molecule binds. Such cell surface receptors include, but are not limited to, Frizzled (Fz), LRP (LRP5 and LRP6), and Kremen (Krm).

As used herein, the term “binding molecule” refers to immunoglobulins and non-immunoglobulin moieties that specifically recognize and bind epitopes of a target molecule.

As used herein a “DKK1/4 binding molecule” is a polypeptide that specifically binds DKK1 or DKK4 or both. In one embodiment, the DKK1/4 binding molecule preferentially binds DKK1 over DKK4 with about a 10-fold to about 1000-fold difference in affinity. In one embodiment, the difference in affinity is 100-fold. In one embodiment, the DKK1/4 binding molecule does not recognize a DKK2 or a DKK3 polypeptide. Examples of a DKK1/4 binding molecule include but are not limited to at least one CDR fragment. Specific CDR fragments of the invention may be in a variety of scaffolds known in the art, including but not limited to, e.g., an antibody or antibody fragment, or an immunoglobulin or non-immunoglobulin moieties that specifically recognize and bind epitopes of the target molecule(s).

As used herein, the term “antibody” refers to immunoglobulins such as polyclonal antibodies, monoclonal antibodies, humanized antibodies, single-chain antibodies, and fragments thereof such as F_{ab}, F_{(ab′)2}, Fᵥ, and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as F_{ab}, F_{(ab′)2}, Fᵥ, and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term “polyclonal antibody” refers to an antibody composition having a heterogeneous antibody population. Polyclonal antibodies are often derived from the pooled serum from immunized animals or from selected humans.

As used herein, the phrase “single chain antibodies” refer to antibodies prepared by determining the binding domains (both heavy and light chains) of a binding antibody, and supplying a linking moiety which permits preservation of the binding function. This forms, in essence, a radically abbreviated antibody, having only that part of the variable domain necessary for binding to the antigen. Determination and construction of single chain antibodies are described in U.S. Pat. No. 4,946,778 to Ladner et al.

A “naturally occurring antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to the protein sequence that binds the target, e.g., one or more CDRs. It includes, e.g., full length antibodies, one or more fragments of an antibody, and/or CDRs on a non-immunoglobulin-related scaffold that retain the ability to specifically bind to an antigen (e.g., DKK1). The antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

As used herein, an “antigen” or an “epitope” interchangeably refer to a polypeptide sequence on a target protein specifically recognized by an antigen-binding portion of an antibody, antibody fragment, a binding molecule or their equivalents. An antigen or epitope comprises at least 6 amino acids, which may be contiguous within a target sequence, or non-contiguous. A conformational epitope may comprise non-contiguous residues, and optionally may contain naturally or synthetically modified amino acid residues. Modifications to residues include, but are not limited to: phosphorylation, glycosylation, PEGylation, ubiquitinization, furanylization, and the like.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85: 5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

As described herein, the conservative variants include amino acid residues in any of the amino acid sequences identified, particularly conservative changes that are well known to one of ordinary skill in the art of protein engineering.

An “isolated antibody”, as used herein, refers to an antibody that is substantially free of other antibodies having different antigenic specificities (e.g., an isolated antibody that specifically binds DKK1 is substantially free of antibodies that specifically bind antigens other than DKK1). An isolated antibody that specifically binds DKK1 may, however, have cross-reactivity to other antigens, such as DKK1 molecules from other species, or other family members such as DKK4 or related paralogs. Moreover, an isolated antibody may be substantially free of other cellular material and/or chemicals.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

As used herein, the term “humanized antibodies” means that at least a portion of the framework regions of an immunoglobulin are derived from human immunoglobulin sequences. A “humanized” antibodies such as antibodies with CDR sequences derived from the germline of another species, especially a mammalian species, e.g., a mouse, that have been grafted onto human framework sequences. Example technologies include humanization technology of PDL.

As used herein, the term “humaneered antibodies” means antibodies that bind the same epitope but differ in sequence. Example technologies include humaneered antibodies produced by humaneering technology of Kalobios, wherein the sequence of the antigen-binging region is derived by, e.g., mutation, rather than due to conservative amino acid replacements.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgA, IgD, IgM, IgE, IgG such as IgG1, IgG2, IgG3 or IgG4) that is provided by the heavy chain constant region genes.

The phrases “an antibody recognizing an antigen” and “an antibody specific for an antigen” are used interchangeably herein with the term “an antibody which binds specifically to an antigen.” As used herein, an antibody that “specifically binds to human DKK1” is intended to refer to an antibody that binds to human DKK1 with a K_{D} of 5×10⁻⁹M or less, 2×10⁻⁹M or less, or 1×10⁻¹⁰ M or less. An antibody that “cross-reacts with an antigen other than human DKK1” is intended to refer to an antibody that binds that antigen with a K_{D} of 0.5×10⁻⁸M or less, 5×10⁻⁹M or less, or 2×10⁻⁹M or less. An antibody that “does not cross-react with a particular antigen” is intended to refer to an antibody that binds to that antigen, with a K_{D} of 1.5×10⁻⁸ M or greater, or a K_{D} of 5-10×10⁻⁸ M or 1×10⁻⁷M or greater. In certain embodiments, such antibodies that do not cross-react with the antigen exhibit essentially undetectable binding against these proteins in standard binding assays.

As used herein, a binding molecule that “inhibits binding of DKK1 to a cell surface receptor” such as LRP, Fz or Krm, refers to an binding molecule that inhibits DKK1 binding to the receptor with a K of 1 nM or less, 0.75 nM or less, 0.5 nM or less, or 0.25 nM or less.

As used herein, “osteolysis” refers to a decrease in bone density, which may be due to various mechanisms of action including, e.g., decreased osteoblast activity, increased osteoclast activity. Osteolysis therefore encompasses mechanisms that generically affect bone mineral density. As used herein, an binding molecule that “inhibits osteolytic activity” is intended to refer to an binding molecule that inhibits loss of bone density either by increasing bone formation or blocking a bone resorption.

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, by FMAT, or by using a biosensor system such as a Biacore® system.

As used herein, the term “affinity” refers to the strength of interaction between a binding molecule, such as an antibody, and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to a measure of the overall stability or strength of the binding molecule-antigen complex. It is controlled by three major factors: binding molecule epitope affinity; the valence of both the antigen and binding molecule; and the structural arrangement of the interacting parts. Ultimately these factors define the specificity of the binding molecule, that is, the likelihood that the particular binding molecule is binding to a precise antigen epitope.

In order to get a higher avidity probe, a dimeric conjugate (two molecules of JWJ-1 coupled to a FACS marker) can be constructed, thus making low affinity interactions (such as with the germline antibody) more readily detected by FACS. In addition, another means to increase the avidity of antigen binding involves generating dimers or multimers of any of the fibronectin constructs described herein of the DKK1 or DKK4 binding molecules. Such multimers may be generated through covalent binding between individual modules, for example, by imitating the natural C-to-N-terminus binding or by imitating antibody dimers that are held together through their constant regions. The bonds engineered into the Fc/Fc interface may be covalent or non-covalent. In addition, dimerizing or multimerizing partners other than Fc can be used in DKK1 or DKK4 hybrids to create such higher order structures.

As used herein, the term “cross-reactivity” refers to an binding molecule or population of binding molecules binding to epitopes on other antigens. This can be caused either by low avidity or specificity of the binding molecule or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” or “high specificity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

As used herein, the term “subject” includes any human or nonhuman animal.

The term “non-human animal” includes all vertebrates, e.g., mammals and non-mammals, such as nonhuman primates, sheep, dogs, cats, horses, cows, chickens, amphibians, reptiles, etc. The term “non-human cell” refers to any cell, eukaryotic or prokaryotic, which is not of human origin, including, inter alia, cells of vertebrate, invertebrate, microbial, fungal or other origin.

As used herein, the term, “optimized” means that a nucleotide sequence has been altered to encode an amino acid sequence using codons that are preferred in the production cell or organism, and/or the nucleotide sequence has been altered to remove latent splice donor or splice acceptor sites. Optimized codon tables are well known in the art for a wide variety of species. Sequences for splice donor and acceptor sites are also known in the art and latent splice sites may be identified, e.g., by analysis of transcript or expression data. Production cells include, but are not limited to, a prokaryotic cell such as e.g., a prokaryotic cell such as a bacterium (E. coli), or a eukaryotic cell, for example, yeast (e.g., Pichia), fungal cell, baculovirus-infected cell, a Chinese Hamster Ovary cell (CHO), a myeloma cell or a human cell. The optimized nucleotide sequence is engineered to retain completely or as much as possible the amino acid sequence and residue number originally encoded by the starting nucleotide sequence, which is also known as the “parental” sequence. The optimized sequences herein have been engineered to have codons that are preferred in the production cells, however optimized expression of these sequences in other eukaryotic and prokaryotic cells is also envisioned herein. The amino acid sequences encoded by optimized nucleotide sequences are optionally referred to as optimized.

In related embodiments, polypeptide sequences of neutralizing anti-DKK1/4 compositions of the invention, and the nucleotides that encode them, are optimized for production and clinical use. Characteristics that may be optimized for clinical use include, but are not limited to, e.g., half-life, pharmacokinetics (PK), antigenicity, effector function, FcRn clearance, and patient response including antibody dependent cell cytotoxicity (ADCC) or complement dependent cytotoxicity (CDC) activities.

As used herein, “DKK1-associated diseases” and/or “DKK4-associated diseases” (“DKK1/4-associated diseases”) include, but are not limited to, osteolytic lesions—especially osteolytic lesions associated with a myeloma (especially a multiple myeloma, MGUS, plateau and smoldering myeloma), or with cancers of the bone, breast, colon, melanocytes, hepatocytes, epithelium, esophagus, brain, lung, prostate or pancreas or metastasis thereof; bone loss associated with transplantation. Further diseases or disorders include but are not limited to, e.g., osteosarcoma, prostate cancer, hepatocellular carcinoma (HCC), myeloma (including multiple myeloma, MGUS, plateau and smoldering myeloma), diabetes, obesity, muscle wasting, Alzheimer's disease, osteoporosis, osteopenia, rheumatism, colitis and/or unwanted hair loss.

As used herein, a “treatment” is an intervention performed with the intention of preventing the development or altering the pathology of a disorder. “Treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other agents, e.g., radiation and/or chemotherapy. The “pathology” of cancer includes all phenomena that compromise the well being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

Treatment of patients suffering from clinical, biochemical, radiological or subjective symptoms of the disease, such as osteolysis, may include alleviating some or all of such symptoms or reducing the predisposition to the disease.

In general, a neutralizing anti-DKK1/4 composition of the invention prevents, treats, or ameliorates Wnt-related diseases associated with DKK1 or DKK4 or both, but not diseases associated with DKK2, DKK3 or with other modulators of the Wnt pathway.

Various aspects of the invention are described in further detail in the following subsections.

The Wnt pathway is a major regulator of mesenchymal stem cell (MSC) differentiation into osteoblasts. It is also an important survival factor for active osteoblasts. Dickkopf-1 (DKK1) is a Wnt pathway antagonist expressed predominantly in bone in adults and is upregulated in myeloma patients with osteolytic lesions. A neutralizing anti-DKK1/4 binding molecule is a truly anabolic agent, which acts through increasing osteoblastic activity while simultaneously decreasing osteoclastic activity. In contrast, current drugs such as PTH, which are marketed as anabolic agents, in fact increase markers associated with both osteoblast ands osteoblasts.

Provided in the invention are polyclonal and monoclonal antibodies selected for binding to DKK1. In one embodiment, an inventive antibody has an affinity of less than 10 pM against human DKK1. In some embodiments, the anti-DKK1 antibody crossreacts with DKK4 (Kd˜300 pM) but not DKK2 (undetectable with current methods).

In one embodiment, an epitope for an anti-DKK1 or anti-DKK4 binding molecule is mapped to the Cys-2 domain (AAs 189-263), which is known to be responsible for both LRP6 and Kremen binding. In one embodiment, the epitope includes at least six, and at most thirty, amino acid residues from the Cys-2 domain of a DKK1 or a DKK4 polypeptide In one embodiment, the epitope includes a stretch of at least six contiguous amino acids. In another embodiments, the binding site is non-linear, i.e., includes non-contiguous amino acid residues. In some embodiments, binding depends on N-glycosylation. Only one N-glycosylation site is predicted, at residue 256 in the Cys-2 domain.

In certain embodiments, a binding molecule of the invention exhibits dose linear pharmacokinetics (AUC) in mice, with a dose dependent terminal half-life of 35-96 hours in mice over a dose of 20-200 μg/mouse.

Accordingly, an binding molecule that “inhibits” one or more of these DKK1 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the binding molecule (e.g., or when a control binding molecule of irrelevant specificity is present). An binding molecule that inhibits DKK1 activity effects such a statistically significant decrease by at least 10% of the measured parameter, by at least 50%, 80% or 90%, and in certain embodiments an binding molecule of the invention may inhibit greater than 95%, 98% or 99% of DKK1 functional activity.

### Dickkopf Family Members

A DKK polypeptide of the invention includes DKK1 (SEQ ID NO:1) and DKK4 (SEQ ID NO:133), as well as DKK2 (SEQ ID NO: 131) and DKK3 (SEQ ID NO:132). DKK family members have two CYS domains (CYS1 and CYS2) as shown in the Table A—DKK1 Family Member PileUp. DKK proteins contains an acid N-terminal signal peptide, two CYS domains containing clusters of cysteine residues separated by a divergent linker region, and a potential C-terminal N-glycosylation site. The CYS2 domain in DKK4 has a lipid-binding function that may facilitate WNT/DKK interactions at the plasma membrane. OMIM accno. 605417.

TABLE A
DKK1 Family Member PileUp

| | |
|---|---|
| hDKK1 | ~~~~~~~~~~ ~~~~~~~~~~ ~~~~~~~~~~ ~~~~~~~~~~ ~~~~~~~~~~ |
| hDKK2 | ~~~~~~~~~~ ~~~~~~~~~~ ~~~~~~~~~~ ~~~~~~~~~~ ~~~~~~~~~~ |
| hDKK3 | MQRLGATLLC LLLAAAVPTA PAPAPTATSA PVKPGPALSY PQEEATLNEM |
| hDKK4 | ~~~~~~~~~~ ~~~~~~~~~~ ~~~~~~~~~~ ~~~~~~~~~~ ~~~~~~~~~~ |
| | 51 100 |
| hDKK1 | ~~~~~~~~~M MALGAAGATR VFVAMVAAAL GGHPLLGVSA TLNSVLNSNA |
| hDKK2 | ~~~~~~~~~M AALMRSKDSS CCLLLLAAVL ....MVESSQ IGSSRAKLNS |
| hDKK3 | FREVEELMED TQHKLRSAVE EMEAEEAAAK ASSEVNLANL PPSYHNETNT |
| hDKK4 | ~~~~~~~~~~ ~~~~~~~~~~ ~~~~~~~~~~ ~~~~~~~~~~ ~~~~~~~~~~ |
| | 101 150 |
| hDKK1 | 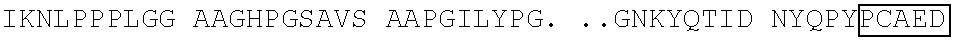 |
| hDKK2 | 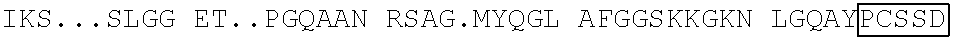 |
| hDKK3 | 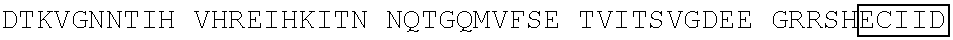 |
| hDKK4 | 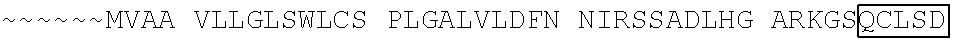 |
| | 151 CYS1 200 |
| hDKK1 | 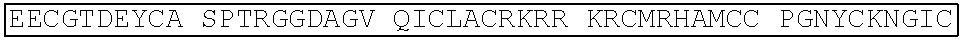 |
| hDKK2 | 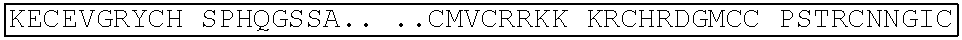 |
| hDKK3 | 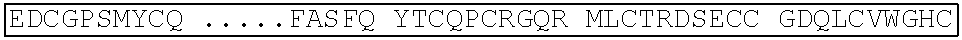 |
| hDKK4 | 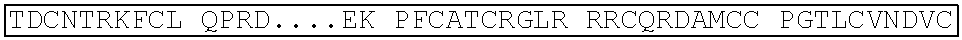 |
| | 201 250 |
| hDKK1 | 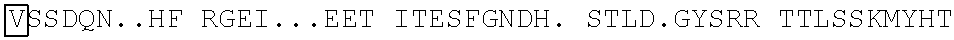 |
| hDKK2 | 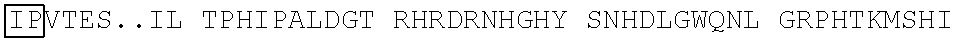 |
| hDKK3 | 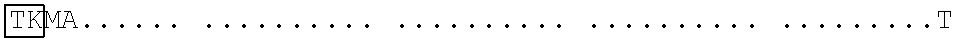 |
| hDKK4 | 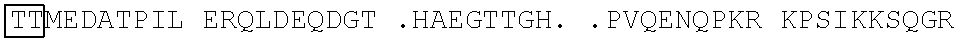 |
| | 251 CYS2 300 |
| hDKK1 | 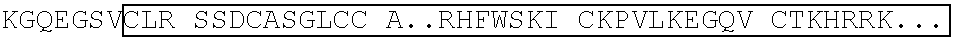 |
| hDKK2 | 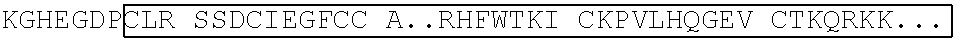 |
| hDKK3 | 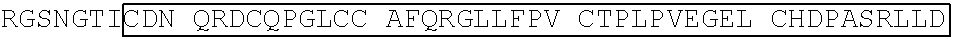 |
| hDKK4 | 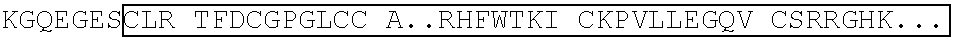 |
| | 301 350 |
| hDKK1 | 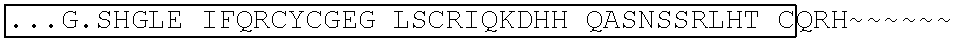 |
| hDKK2 | 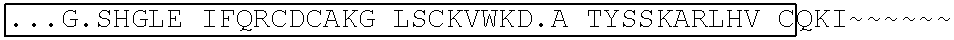 |
| hDKK3 | 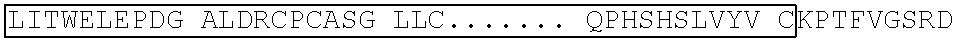 |
| hDKK4 | 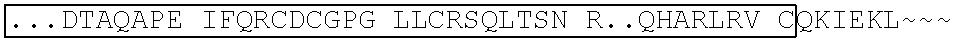 |
| | 351 400 |
| hDKK1 | ~~~~~~~~~~ ~~~~~~~~~~ ~~~~~~~~~~ ~~~~~~~~~~ ~~~~~~~~~~ |
| hDKK2 | ~~~~~~~~~~ ~~~~~~~~~~ ~~~~~~~~~~ ~~~~~~~~~~ ~~~~~~~~~~ |
| hDKK3 | QDGEILLPRE VPDEYEVGSF MEEVRQELED LERSLTEEMA LGEPAAAAAA |
| hDKK4 | ~~~~~~~~~~ ~~~~~~~~~~ ~~~~~~~~~~ ~~~~~~~~~~ ~~~~~~~~~~ |
| | 401 |
| hDKK1 | ~~~~~~~ (SEQ ID NO: 1) |
| hDKK2 | ~~~~~~~ (SEQ ID NO: 131) |
| hDKK3 | LLGGEEI (SEQ ID NO: 132) |
| hDKK4 | ~~~~~~~ (SEQ ID NO: 133) |

### Binding Molecules Against DKK1 and DKK4

In one embodiment, the binding molecule of the invention is specific to a human DKK protein. In one embodiment, the binding molecule of the invention is specific to a human DKK1 or DKK4 protein, or both.

A DKK1 or DKK4 neutralizing binding molecule is distinct from the Wnt pathway modifications that have been linked to tumor promotion. The Wnt pathway is regulated by a complex network of extracellular ligands, receptors and antagonists of which DKK1 is only one. Due to the restricted expression of DKK1 in adults and its functional redundancy with other Wnt antagonists, a neutralizing DKK1 binding molecule is unlikely to cause widespread activation of Wnt signaling or therefore, tumorigenesis. This is further supported by two observations: first, activating LRP5 mutations (inhibiting DKK binding) induce a high bone mass phenotype but have no apparent increased cancer risk [Moon 2004], while DKK1 heterozygous null or Doubleridge mice have decreased DKK1 levels, high bone mass phenotype, but no reported increased rate of tumor formation [MacDonald 2004].

An anti-DKK1 binding molecule should positively impact myeloma-induced osteolytic disease while not increasing the risk of de novo tumorigenesis. It is expected that such a binding molecule would be used in combination with anti-tumor chemotherapies and possibly with anti-bone resorption drugs that inhibit osteoclast function. Other therapeutic combinations are provided herein.

A binding molecule that neutralizes DKK1, DKK4 or both may be an antibody.

### Polyclonal Antibodies

Antibodies of the invention may be polyclonal antibodies, especially human polyclonal antibodies. Polyclonals are derived from pooled serum from immunized animals or selected humans.

### Monoclonal Antibodies

In one embodiment, antibodies of the invention are the human monoclonal antibodies, such as the isolated and structurally characterized, e.g., in Examples 1-8. Specific V_{H} amino acid sequences of the antibodies are shown, e.g., in SEQ ID NOs: 2-20. Specific V_{L} amino acid sequences of the antibodies are shown, e.g., in SEQ ID NOs: 21-39.

A V_{H} amino acid sequence of the antibody may be optimized for expression in a mammalian cell, e.g., such as the sequence shown in SEQ ID NO: 124. A V_{L} amino acid sequence of the antibodies may be optimized for expression in a mammalian cell, e.g., such as the sequence shown in SEQ ID NO: 121. Likewise, sequences may be optimized for expression in, e.g., yeast, bacteria, hamster and other cells, depending on which expression system is preferred for the characteristic being optimized. Other antibodies of the invention include amino acids that have been mutated, yet have at least 60, 70, 80, 90, 95, 96, 97, 98 or 99 percent identity in the CDR regions with the CDR regions depicted in the sequences described above.

In one embodiment, full length optimized light chain parental nucleotide sequences are as shown in SEQ ID NOs: 99, 101, 103 and 105. Full length optimized heavy chain parental nucleotide sequences are as shown in SEQ ID NOs: 107, 109 and 111. Such full length LC and HC nucleotide sequences may be further optimized for expression in mammalian cells. Full length light chain amino acid sequences encoded by these optimized light chain parental nucleotide sequences are as shown in SEQ ID NOs: 100, 102, 104 and 106. Full length heavy chain amino acid sequences encoded by these optimized heavy chain parental nucleotide sequences are as shown in SEQ ID NOs: 108, 110 and 112. Other antibodies of the invention include amino acids or nucleic acids that have been mutated, yet have at least 60, 70, 80, 90, 95, 96, 97, 98 or 99 percent identity to the inventive sequences described herein and above.

Since each of these antibodies can bind to DKK1, the V_{H}, V_{L}, full length light chain, and full length heavy chain sequences (nucleotide sequences and amino acid sequences) can be “mixed and matched” to create other anti-DKK1 binding molecules of the invention. DKK1 binding of such “mixed and matched” antibodies can be tested using the binding assays described above and in the Examples (e.g., ELISAs). When these chains are mixed and matched, a V_{H} sequence from a particular V_{H}/V_{L} pairing should be replaced with a structurally similar V_{H} sequence. Likewise a full length heavy chain sequence from a particular full length heavy chain/full length light chain pairing should be replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. The V_{H}, V_{L}, full length light chain, and full length heavy chain sequences of the antibodies of the present invention are particularly amenable for mixing and matching, since these antibodies use V_{H}, V_{L}, full length light chain, and full length heavy chain sequences derived from the same germline sequences and thus exhibit structural similarity.

Accordingly, in one aspect, the invention provides an isolated monoclonal antibody or antigen binding portion thereof having: a V_{H} region comprising an amino acid sequence selected from the group consisting of SEQ ID NOs: 2-20 and 124; and a V_{L} region comprising an amino acid sequence selected from the group consisting of SEQ ID NOs: 21-39 and 121; wherein the antibody specifically binds DKK1.

Examples of heavy and light chain combinations include: a V_{H} region comprising the amino acid sequence of SEQ ID NO: 2 and a V_{L} region comprising the amino acid sequence of SEQ ID NO: 21; or a V_{H} region comprising SEQ ID NO: 3 and a V_{L} region comprising SEQ ID NO: 22; or a V_{H} region comprising SEQ ID NO: 4 and a V_{L} region comprising SEQ ID NO: 23; or a V_{H} region comprising SEQ ID NO: 5 and a V_{L} region comprising SEQ ID NO: 24; or a V_{H} region comprising SEQ ID NO: 6 and a V_{L} region comprising SEQ ID NO: 25; or a V_{H} region comprising SEQ ID NO: 7 and a V_{L} region comprising SEQ ID NO: 28; or a V_{H} region comprising SEQ ID NO: 8 and a V_{L} region comprising SEQ ID NO: 29; or a V_{H} region comprising SEQ ID NO: 9 and a V_{L} region comprising SEQ ID NO: 30; or a V_{H} region comprising SEQ ID NO: 10 and a V_{L} region comprising SEQ ID NO: 31; or a V_{H} region comprising SEQ ID NO: 11 and a V_{L} region comprising SEQ ID NO: 32; or a V_{H} region comprising SEQ ID NO: 12 and a V_{L} region comprising SEQ ID NO: 33; or a V_{H} region comprising SEQ ID NO: 124 and a V_{L} region comprising SEQ ID NO: 121.

In another aspect, the invention provides an isolated monoclonal antibody or antigen binding portion thereof having: a full length heavy chain comprising an amino acid sequence selected from the group consisting of SEQ ID NOs: 108, 110 and 112; and a full length light chain comprising an amino acid sequence selected from the group consisting of SEQ ID NOs: 100, 102, 104 and 106.

Thus, examples of full length heavy chain and full length light chain combinations, respectively, include: SEQ ID NO: 108 with SEQ ID NO: 100; or SEQ ID NO: 110 with SEQ ID NO: 102; or SEQ ID NO: 112 with SEQ ID NO: 104; or SEQ ID NO: 112 with SEQ ID NO: 106.

In another aspect, the invention provides an isolated monoclonal antibody or antigen binding portion thereof comprising a full length heavy chain encoded by a nucleotide sequence selected from the group consisting of SEQ ID NOs: 107, 109 and 111; and a full length optimized light chain encoded by a nucleotide sequence selected from the group consisting of SEQ ID NOs: 99, 101, 103 and 105.

Thus, examples of nucleotides that encode full length heavy and light chains, respectively, that may be combined include: SEQ ID NO: 107 and 99; or SEQ ID NO: 109 and 101; or a SEQ ID NO: 111 and 103; or SEQ ID NO: 111 and 105.

In yet another aspect, the invention provides antibodies that comprise the heavy chain and light chain CDR1s, CDR2s and CDR3s of the antibodies, or combinations thereof. The amino acid sequences of the V_{H} chains of the inventive antibodies are shown in SEQ ID NOs: 2-20. Their respective V_{H} CDR1 amino acid sequences are provided as SEQ ID NOs: 49-52. Their respective V_{H} CDR2 amino acid sequences are provided as SEQ ID NOs: 53-63. Their respective V_{H} CDR3 amino acid sequences are provided as SEQ ID NOs: 64-69. The amino acid sequences of the V_{L} kappa and lambda light chains of the inventive antibodies are shown in SEQ ID NOs: 21-39. Their respective V_{L} CDR1 amino acid sequences are provided as SEQ ID NOs: 70-74. Their respective V_{L} CDR2 amino acid sequences are provided as SEQ ID NOs: 75-79. Their respective V_{L} CDR3 amino acid sequences are provided as SEQ ID NOs: 80-98. The CDR regions are delineated using the Kabat system (Kabat, E. A., et al., 1991 Sequences of Proteins of Immunological Interest, Fifth Edition, U.S. Department of Health and Human Services, NIH Publication No. 91-3242).

Given that each of these antibodies can bind to DKK1 and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and match, although each antibody must contain a V_{H} CDR1, 2 and 3 and a V_{L} CDR1, 2 and 3 to create other anti-DKK1 binding molecules of the invention. DKK1 binding of such “mixed and matched” antibodies can be tested using the binding assays described above and in the Examples (e.g., ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). Furthermore, CDR1, CDR2 and/or CDR3 sequence from a particular V_{H} or V_{L} sequence may be specifically or randomly mutated to create antibodies that may be tested for affinity or binding characteristics. It will be readily apparent to the ordinarily skilled artisan that novel V_{H} and V_{L} sequences can be created by substituting one or more V_{H} and/or V_{L} CDR region sequences with structurally similar sequences from the CDR sequences shown herein for monoclonal antibodies of the present invention.

An isolated monoclonal antibody, or antigen binding portion thereof has: a V_{H} region CDR1 comprising an amino acid sequence selected from the group consisting of SEQ ID NOs: 2-5, 8-11, 20 are provided as 49-52; a V_{H} region CDR2 comprising an amino acid sequence selected from the group consisting of SEQ ID NOs: 2-20 are provided as SEQ ID NOs: 53-63; a V_{H} region CDR3 comprising an amino acid sequence selected from the group consisting of SEQ ID NOs: 2-20 are provided as SEQ ID NOs: 64-69; a V_{L} region CDR1 comprising an amino acid sequence selected from the group consisting of SEQ ID NOs: 21-39 are provided as SEQ ID NOs: 70-74; a V_{L} region CDR2 comprising an amino acid sequence selected from the group consisting of SEQ ID NOs: 21-39 are provided as SEQ ID NOs: 75-79; and a V_{L} region CDR3 comprising an amino acid sequence selected from the group consisting of SEQ ID NOs: 21-39 are provided as SEQ ID NOs:80-98; wherein the antibody specifically binds DKK1.

In one embodiment, the inventive antibody consists of: a V_{H} region CDR3 comprising SEQ ID NO: 69 and a V_{L} region CDR3 comprising SEQ ID NO: 80.

In one embodiment, the inventive antibody consists of: a V_{H} region CDR3 comprising SEQ ID NO: 64 and a V_{L} region CDR3 comprising SEQ ID NO: 81.

In one embodiment, the inventive antibody consists of: a V_{H} region CDR3 comprising SEQ ID NO: 65 and a V_{L} region CDR3 comprising SEQ ID NO: 82.

In one embodiment, the inventive antibody consists of: a V_{H} region CDR3 comprising SEQ ID NO: 66 and a V_{L} region CDR3 comprising SEQ ID NO: 87.

In one embodiment, the inventive antibody consists of: a V_{H} region CDR3 comprising SEQ ID NO: 67 and a V_{L} region CDR3 comprising SEQ ID NO: 92.

In one embodiment, the inventive antibody consists of: a V_{H} region CDR3 comprising SEQ ID NO: 68 and a V_{L} region CDR3 comprising SEQ ID NO: 98.

As used herein, a human antibody comprises heavy or V_{L} regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody of the invention has full length heavy and light chain amino acid sequences; full length heavy and light chain nucleotide sequences, variable region heavy and light chain nucleotide sequences, or variable region heavy and light chain amino acid sequences that are homologous to the amino acid and nucleotide sequences of the antibodies described herein, and wherein the antibodies retain the desired functional properties of the neutralizing anti-DKK1/4 composition of the Invention.

For example, the invention provides an isolated monoclonal antibody, or antigen binding portion thereof, comprising a V_{H} region and a V_{L} region, wherein: the V_{H} region comprises an amino acid sequence that is at least 80% homologous to an amino acid sequence selected from the group consisting of SEQ ID NOs: 2-20 and 124; the V_{L} region comprises an amino acid sequence that is at least 80% homologous to an amino acid sequence selected from the group consisting of SEQ ID NOs: 21-39 and 121; the antibody specifically binds to DKK1 and/or DKK4, and the antibody exhibits at least one of the following functional properties: the antibody neutralizes binding of a DKK1 protein to LRP6, Fz and/or Krm, or the antibody neutralizes binding of a DKK4 protein to LRP, Pz and/or Krm.

In a further example, the invention provides an isolated monoclonal antibody, or antigen binding portion thereof, comprising a full length heavy chain and a full length light chain, wherein: the full length heavy chain comprises an amino acid sequence that is at least 80% homologous to an amino acid sequence selected from the group consisting of SEQ ID NOs: 108, 110 and 112; the full length light chain comprises an amino acid sequence that is at least 80% homologous to an amino acid sequence selected from the group consisting of SEQ ID NOs: 100, 102, 104 and 106; the antibody specifically binds to DKK1, and the antibody exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In another example, the invention provides an isolated monoclonal antibody, or antigen binding portion thereof, comprising a full length heavy chain and a full length light chain, wherein: the full length heavy chain comprises a nucleotide sequence that is at least 80% homologous to a nucleotide sequence selected from the group consisting of SEQ ID NOs: 107, 109 and 111; the full length light chain comprises a nucleotide sequence that is at least 80% homologous to a nucleotide sequence selected from the group consisting of SEQ ID NOs: 99, 101, 103 and 105; the antibody specifically binds to DKK1 and exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In another example, the invention provides an isolated monoclonal antibody, or antigen binding portion thereof that has been optimized for expression in a cell, comprising a full length heavy chain and a full length light chain, wherein: the full length heavy chain comprises a nucleotide sequence that is at least 80% homologous to a nucleotide sequence selected from the group consisting of SEQ ID NOs: 108-110; the full length light chain comprises a nucleotide sequence that is at least 80% homologous to a nucleotide sequence selected from the group consisting of SEQ ID NOs: 104-107; the antibody specifically binds to DKK1, and the antibody exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In another example, the invention provides an isolated monoclonal antibody, or antigen binding portion thereof, that has been optimized for expression in a cell, comprising a V_{H} region and a V_{L} region, wherein: the full length heavy chain comprises a nucleotide sequence that is at least 80% homologous to a nucleotide sequence selected from the group consisting of SEQ ID NO: 121; the full length light chain comprises a nucleotide sequence that is at least 80% homologous to a nucleotide sequence selected from the group consisting of SEQ ID NO: 120; the antibody specifically binds to DKK1, and the antibody exhibits at least one of the following properties: the antibody inhibits binding DKK1 to the DKK1 receptor or the antibody inhibits DKK1 receptor, binding preventing or ameliorating osteolysis or the antibody inhibits DKK1 receptor binding, preventing or ameliorating osteolytic lesions or the antibody inhibits DKK1 receptor binding, preventing or ameliorating cancer.

As used herein, the percent homology between two amino acid sequences or two nucleotide sequences is equivalent to the percent identity between the two sequences. The percent identity between the two sequences is a function of the number of identical positions shared by the sequences (i.e., % homology equals # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., 1990 J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 Nucleic Acids Res. 25(17):3389-3402. When utilizing BLAST and Gapped BLAST programs, the default parameters of the respective programs (e.g., XBLAST and NBLAST) can be used. See http:www.ncbi.nhn.nih.gov.

Antibodies with Conservative Modifications

In certain embodiments, an antibody of the invention has a V_{H} region consisting of CDR1, CDR2, and CDR3 sequences and a V_{L} region consisting of CDR1, CDR2, and CDR3 sequences, wherein one or more of these CDR sequences have specified amino acid sequences based on the antibodies described herein or conservative modifications thereof, and wherein the antibodies retain the desired functional properties of the neutralizing anti-DKK1/4 composition of the invention. Accordingly, the invention provides an isolated monoclonal antibody, or antigen binding portion thereof, consisting of a V_{H} region consisting of CDR1, CDR2, and CDR3 sequences, and a V_{L} region consisting of CDR1, CDR2, and CDR3 sequences, wherein: the CDR1 sequence of the V_{H} variable region is selected from the group consisting of SEQ ID NOs: 49-52, and conservative modifications thereof; the CDR2 sequence of the V_{H} variable region is selected from the group consisting of SEQ ID NOs:53-63, and conservative modifications thereof; the CDR3 sequence of the V_{H} variable region is selected from the group consisting of SEQ ID NOs: 64-69, and conservative modifications thereof; the CDR1 sequence of the V_{L} variable region is selected from the group consisting of SEQ ID NOs: 70-74, and conservative modifications thereof; the CDR2 sequence of the V_{L} variable region is selected from the group consisting of SEQ ID NOs: 75-79, and conservative modifications thereof; the CDR3 sequence of the V_{L} variable region is selected from the group consisting of 80-98, and conservative modifications thereof; the antibody specifically binds to DKK1; and the antibody exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In other embodiments, an antibody of the invention has a full length heavy chain sequence and a full length light chain sequence, wherein one or more of these sequences have specified amino acid sequences based on the antibodies described herein or conservative modifications thereof, and wherein the antibodies retain the desired functional properties of the neutralizing anti-DKK1/4 composition of the invention. Accordingly, the invention provides an isolated monoclonal antibody, or antigen binding portion thereof, consisting of a full length heavy chain and a full length light chain wherein: the full length heavy chain has amino acid sequences selected from the group of SEQ ID NOs: 108, 110 and 112, and conservative modifications thereof; and the full length light chain has amino acid sequences selected from the group of SEQ ID NOs: 100, 102, 104 and 106, and conservative modifications thereof; the antibody specifically binds to DKK1; and the antibody exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In other embodiments, an antibody of the invention optimized for expression in a cell has a V_{H} region sequence and a V_{L} region sequence, wherein one or more of these sequences have specified amino acid sequences based on the antibodies described herein or conservative modifications thereof, and wherein the antibodies retain the desired functional properties of the neutralizing anti-DKK1/4 composition of the invention. Accordingly, the invention provides an isolated monoclonal antibody, or antigen binding portion thereof, consisting of a V_{H} region and a V_{L} region wherein: the V_{H} region has amino acid sequences selected from the group of SEQ ID NO: 124, and conservative modifications thereof; and the V_{L} region has amino acid sequences selected from the group of SEQ ID NOs: 121, and conservative modifications thereof; the antibody specifically binds to DKK1; and the antibody exhibits at least one of the following functional properties: the antibody inhibits binding DKK1 protein to the DKK1 receptor or the antibody inhibits DKK1 receptor binding preventing or ameliorating osteolysis or the antibody inhibits DKK1 receptor binding preventing or ameliorating osteolytic lesions or the antibody inhibits DKK1 receptor binding preventing or ameliorating cancer.

In various embodiments, the antibody may exhibit one or more, two or more, or three or more of the functional properties listed discussed herein. Such antibodies can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis.

Conservative amino acid substitutions are ones in which the amino acid residue is replaced by an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family, and the altered antibody can be tested for retained function using the functional assays described herein.

Antibodies that Bind to the Same Epitope as Neutralizing Anti-DKK1/4 Composition of the Invention

In another embodiment, the invention provides antibodies that bind to the same epitope as the various neutralizing anti-DKK1/4 composition of the invention provided herein. Such additional antibodies can be identified based on their ability to cross-compete (e.g., to competitively inhibit the binding of, in a statistically significant manner) with other antibodies of the invention in standard DKK1 binding assays. The ability of a test antibody to inhibit the binding of antibodies of the present invention to human DKK1 demonstrates that the test antibody can compete with that antibody for binding to human DKK1; such an antibody may, according to non-limiting hypotheses, bind to the same or a related (e.g., a structurally similar or spatially proximal) epitope on human DKK1 as the antibody with which it competes. In a certain embodiment, the antibody that binds to the same epitope on human DKK1 as the antibodies of the present invention is a human monoclonal antibody. Such human monoclonal antibodies can be prepared and isolated as described in the Examples.

### Camelid and Other Heavy Chain Antibodies

Antibody proteins obtained from members of the camel and dromedary (Camelus bactrianus and Camelus dromaderius) family including new world members such as llama species (Lama paccos, Lama glama and Lama vicugna) have been characterized with respect to size, structural complexity and antigenicity for human subjects. Certain IgG antibodies from this family of mammals lack light chains. They are thus structurally distinct from the typical four chain quaternary structure (having two heavy and two light chains), found in antibodies from other animals. See PCT/EP93/02214 (WO 94/04678 published 3 Mar. 1994).

A region of the camelid antibody which is the small single variable domain identified as V can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for DKK1. In certain embodiments herein, the camelid antibody or nanobody is naturally produced in the camelid animal, i.e., is produced by the camelid following immunization with DKK1 or a peptide fragment thereof, using techniques described herein for other antibodies. Alternatively, the neutralizing anti-DKK1/4 camelid nanobody is engineered, i.e., produced by selection for example from a library of phage displaying appropriately mutagenized camelid nanobody proteins using panning procedures with DKK1 and/or DKK4 as a target as described in the examples herein. Engineered nanobodies can further be customized by genetic engineering to have a half life in a recipient subject of from 45 minutes to two weeks.

In addition to Camelid antibodies, heavy chain antibodies occur naturally in other animal including but not limited to, e.g., certain species of shark and pufferfish (see, e.g., PCT publication WO 03/014161). Although variable domains derived from such heavy chain antibodies may be used in the invention, the use of Camelid-derived heavy chain antibodies and/or of the variable domain sequences thereof is preferred optimization, humanization, humaneering, and the like and/or for clinical use in humans.

### Engineered and Modified Antibodies

An antibody of the invention further can be prepared using an antibody having one or more of the V_{H} and/or V_{L} sequences shown herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad. See. U.S.A. 86:10029-10033; U.S. Pat. No. 5,225,539 to winter, and U.S. Pat. Nos. 5,530,101; 5,585,089; 5,693,762 and 6,180,370 to Queen et al.)

Accordingly, another embodiment of the invention pertains to an isolated monoclonal antibody, or antigen binding portion thereof, comprising a V_{H} region comprising a CDR1 region having an amino acid sequence selected from the group consisting of SEQ ID NOs: 49-52 and 40-43; a CDR2 region having an amino acid sequence selected from the group consisting of SEQ ID NOs: 53-63 and 44-47; a CDR3 region having an amino acid sequence selected from the group consisting of SEQ ID NOs: 64-69 and 48, respectively; and a V_{L} region comprising a CDR1 region having an amino acid sequence selected from the group consisting of SEQ ID NOs: 70-74, 113 and 116; a CDR2 region having an amino acid sequence selected from the group consisting of SEQ ID NOs: 75-79, 114 and 117; and a CDR3 region consisting of an amino acid sequence selected from the group consisting of SEQ ID NOs: 80-98, 115 and 118, respectively. Thus, such antibodies contain the V_{H} and V_{L} CDR sequences of monoclonal antibodies, yet may contain different framework sequences from these antibodies.

Such framework sequences can be obtained from public DNA databases or published references that include germline antibody gene sequences. For example, germline DNA sequences for human heavy and V_{L} region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference.

An example of framework sequences for use in the antibodies of the invention are those that are structurally similar to the framework sequences used by selected antibodies of the invention, e.g., consensus sequences and/or framework sequences used by monoclonal antibodies of the invention. The V_{H} CDR1, 2 and 3 sequences, and the V_{L} CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Accordingly, in another embodiment, the invention provides isolated neutralizing anti-DKK1/4 composition, or antigen binding portions thereof, consisting of a V_{H} region having: a V_{H} CDR1 region consisting of an amino acid sequence selected from the group of SEQ ID NOs: 49-52, or an amino acid sequence having one, two, three, four or five amino acid substitutions, deletions or additions as compared to SEQ ID NOs: 49-52; a V_{H} CDR2 region having an amino acid sequence selected from the group consisting of SEQ ID NOs: 53-63, or an amino acid sequence having one, two, three, four or five amino acid substitutions, deletions or additions as compared to SEQ ID NOs: 53-63; a V_{H} CDR3 region having an amino acid sequence selected from the group consisting of SEQ ID NOs: 64-69, or an amino acid sequence having one, two, three, four or five amino acid substitutions, deletions or additions as compared to SEQ ID NOs: 64-69; a V_{L} CDR1 region having an amino acid sequence selected from the group consisting of SEQ ID NOs: 70-74, or an amino acid sequence having one, two, three, four or five amino acid substitutions, deletions or additions as compared to SEQ ID NOs: 70-74; a V_{L} CDR2 region having an amino acid sequence selected from the group consisting of SEQ ID NOs: 75-79, or an amino acid sequence having one, two, three, four or five amino acid substitutions, deletions or additions as compared to SEQ ID NOs: 75-79; and a V_{L} CDR3 region having an amino acid sequence selected from the group consisting of SEQ ID NOs: 80-98, or an amino acid sequence having one, two, three, four or five amino acid substitutions, deletions or additions as compared to SEQ ID NOs: 80-98.

Engineered antibodies of the invention include those in which modifications have been made to framework residues within V_{H} and/or V_{L}, e.g. to improve the properties of the antibody. Typically such framework modifications are made to decrease the immunogenicity of the antibody. For example, one approach is to “back mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis. Such “back mutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et at.

In another embodiment, one or more amino acid residues are altered to thereby alter the ability of the antibody to fix complement. This approach is described further in PCT Publication WO 94/29351 by Bodmer et at.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604).

In still another embodiment, the glycosylation of an antibody is modified. For example, an aglycoslated antibody can be made (i.e., the antibody lacks glycosylation). Glycosylation can be altered to, e.g., increase the affinity of the antibody for an “antigen’. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Methods of Engineering Antibodies

As discussed above, the anti-DKK1 antibodies having V_{H} and V_{L} sequences or full length heavy and light chain sequences shown herein can be used to create new anti-DKK1/4 antibodies by modifying full length heavy chain and/or light chain sequences, V_{H} and/or V_{L} sequences, or the constant region(s) attached thereto. Thus, in another aspect of the invention, the structural features of an anti-DKK1 antibody of the invention are used to create structurally related anti-DKK1/4 antibodies that retain at least one functional property of the antibodies of the invention, such as binding to human DKK1 or DKK4 or both and also inhibiting one or more functional properties of DKK1 or DKK4 or both.

Standard molecular biology techniques can be used to prepare and express the altered antibody sequence. The antibody encoded by the altered antibody sequence(s) is one that retains one, some or all of the functional properties of the neutralizing anti-DKK1/4 compositions described herein, which functional properties include, but are not limited to, specifically binding to human DKK1; and the antibody exhibits at least one of the following functional properties: the antibody inhibits binding of DKK1 protein to the DKK1 receptor, or the antibody inhibits DKK1 receptor binding preventing or ameliorating osteolysis, or the antibody inhibits DKK1 receptor binding thereby preventing or ameliorating osteolytic lesions, or the antibody inhibits DKK1 receptor binding preventing or ameliorating cancer.

The functional properties of the altered antibodies can be assessed using standard assays available in the art and/or described herein, such as those set forth in the Examples (e.g., ELISAs).

In certain embodiments of the methods of engineering antibodies of the invention, mutations can be introduced randomly or selectively along all or part of an anti-DKK1 antibody coding sequence and the resulting modified anti-DKK1 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

The Fc constant region of an antibody is critical for determining serum half-life and effector functions, i.e., antibody dependent cell cytotoxicity (ADCC) or complement dependent cytotoxicity (CDC) activities. One can engineer specific mutants of the Fc fragment to alter the effector function and/or serum half-life (see, e.g., Xencor technology, see also, e.g., WO2004029207).

One method to alter effector function and serum half-life of an antibody is to graft the variable region of an antibody fragment with an Fc fragment having the appropriate effector function. IgG1 or IgG4 isotypes can be selected for cell killing activity, whereas IgG2 isotype can be used for silent or neutralizing antibodies (with no cell killing activity).

Silent antibodies with long serum half-life can be obtained by making chimeric fusion of variable regions of an antibody with a serum protein such as HSA or a protein binding to such serum protein, such HSA-binding protein.

Effector functions can also be altered by modulating the glycosylation pattern of the antibody. Glycart (e.g., U.S. Pat. No. 6,602,684), Biowa (e.g., U.S. Pat. No. 6,946,292) and Genentech (e.g WO03/035835) have engineered mammalian cell lines to produce antibodies with increased or decreased effector function. Especially, non fucosylated antibodies will have enhanced ADCC activities. Glycofi has also developed yeast cell lines capable of producing specific glycoforms of antibodies.

A more complete disclosure may be found in WO2007/084344 to Shulok et al.

### Nucleic Acid Molecules Encoding Antibodies of the Invention

Another aspect of the invention pertains to nucleic acid molecules that encode the antibodies of the invention. Examples of full length light chain parental nucleotide sequences may be found in WO2007/084344 to Shulok et al.

### Production of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

Hybridoma, chimeric or humanized antibodies of the present invention can be prepared as provided in WO2007/084344 to Shulok et al.

### Pharmaceutical Compositions

In another aspect, the present invention provides a composition, e.g., a pharmaceutical composition, containing one or a combination of a neutralizing anti-DKK1/4 composition, or antigen-binding portion(s) thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e., combined with other agents.

In one embodiment, the DKK1/4 binding molecule of the invention is administered in combination with zoledronic acid.

In one embodiment, the combination therapy can include an anti-DKK1 antibody of the present invention combined with at least one other anti-inflammatory or anti-osteoprotic agent or with a bone anabolic, a weight loss therapy and/or a diabetes therapy. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion), or injection directly into lytic lesions, e.g., in bone. Depending on the route of administration, the active compound, i.e., antibody, immunoconjuage, or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures known in the art, e.g., radiation, filtration, or the inclusion of antibacterial and antifungal agents, for example, paraben, chlorobutanol, phenol sorbic acid. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the antibody, the dosage ranges from about 0.0001 to 200 mg/kg, and more usually about 0.01 to about 50 mg/kg, of the host body weight. For example, dosages can be at least about 0.3 mg/kg body weight, 1 mg/kg body weight, 3 mg/kg body weight, 5 mg/kg body weight or 10 mg/kg body weight or 40 mg/kg body weight within the range of 1-100 mg/kg body weight, and/or less than about 0.3, 1, 3, 5, 10, 20, 40, 50 or 60 mg/kg body weight. An exemplary treatment regime entails administration once per week, once every two weeks, once every three weeks, once every four weeks, once a month, once every 3 months or once every three to 6 months. Dosage regimens for an anti-DKK1/4 antibody of the invention include 1 mg/kg body weight or 3 mg/kg body weight or 40 mg/kg by intravenous administration, with the antibody being given using one of the following dosing schedules: every four weeks for six dosages, then every three months; every three weeks; 3 mg/kg body weight once followed by 1 mg/kg body weight every three weeks: or 40 mg/kg body weight every 28 days. The final dose and dosing regimen is optimized depending on outcome, e.g. bone strengthing and/or anti-tumor effect, and is within the knowledge of one skilled in the art without recourse to undue experimentation.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring patient blood levels of antibody to the target antigen or of some biomarker such as OCN, OPG or P1NP. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-DKK1 antibody of the invention can results in a decrease in severity of disease symptoms, an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, an antibody of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art, such as the devices shown in U.S. Pat. Nos. 5,399,163; 5,383,851; 5,312,335; 5,064,413; 4,941,880; 4,790,824; 4,596,556; 4,487,603; 4,486,194; 4,447,233; 4,447,224; 4,439,196; and 4,475,196. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol. 1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### The Combinations

The additional therapeutic agent can be selected from the group consisting of an anti-cancer agent; an anti-osteoporotic agent; an antibiotic; an antimetabolic agent; an anti-inflammatory agent; an anti-angiogenic agent; a growth factor; a bone anabolic, a weight loss therapy, an antidiabetic agent, a hypylipidemic agent, and anti-obesity agent, an anti-hypertensive agent, and/or an agonist of peroxisome proliferators-activator receptors (PPARs) and a cytokine.

The invention further relates to a method of preventing or treating a DKK1-, DKK4- or DKK1/4-associated disease or disorder in a mammal, particularly a human, with a combination of pharmaceutical agents that comprises:

(a) a DKK1/4 binding molecule of the invention; and

(b) one or more pharmaceutically active agents; and optionally

(c) a pharmaceutically acceptable carrier;

wherein at least one pharmaceutically active agent is an anti-cancer therapeutic.

The invention further relates to pharmaceutical compositions comprising:

(a) a DKK1/4 neutralizing agent; and

(b) a pharmaceutically active agent; and optionally

(c) a pharmaceutically acceptable carrier;

wherein at least one pharmaceutically active agent is a bone anabolic, a weight loss therapeutic or a diabetes therapeutic.

The present invention further relates to a commercial package or product comprising:

(a) a pharmaceutical formulation of a DKK1/4 neutralizing binding molecule; and

(b) a pharmaceutical formulation of a pharmaceutically active agent for simultaneous, concurrent, separate or sequential use;

wherein at least one pharmaceutically active agent is an anti-cancer therapeutic, a bone anabolic, a weight loss therapeutic or a diabetes therapeutic.

The additional therapeutic agent can be selected from the group consisting of an anti-cancer agent; an anti-osteoporotic agent; an antibiotic; an antimetabolic agent; an antidiabetic agent; an anti-inflammatory agent; an anti-angiogenic agent; a growth factor; a bone anabolic, a weight loss therapy, a hypylipidemic agent, and anti-obesity agent, an anti-hypertensive agent, and/or an agonist of peroxisome proliferators-activator receptors (PPARs) and a cytokine, or any one or more of the pharmaceutically active agents provided herein.

### The Pharmaceutically Active Agents

The term “pharmaceutically active agents” is a broad one covering many pharmaceutically active agents having different mechanisms of action. Combinations of some of these with DKK1/4 neutralizing antibodies/compositions can result in improvements in cancer therapy. Generally, pharmaceutically active agents are classified according to the mechanism of action. Many of the available agents are anti-metabolites of development pathways of various tumors, or react with the DNA of the tumor cells. There are also agents which inhibit enzymes, such as topoisomerase I and topoisomerase II, or which are anti-mitotic agents. Further agents are provided for treatment of non-neoplastic diseases associated with DKK1, DKK4 or both.

By the term “pharmaceutically active agent” is meant especially any pharmaceutically active agent other than a neutralizing anti-DKK1/4 composition or a derivative thereof. It includes, but is not limited to:

1. an aromatase inhibitor;

2. an anti-estrogen, an anti-androgen or a gonadorelin agonist;

3. a topoisomerase I inhibitor or a topoisomerase II inhibitor;

4. a microtubule active agent, an alkylating agent, an anti-neoplastic anti-metabolite or a platin compound;

5. a compound targeting/decreasing a protein or lipid kinase activity or a protein or lipid phosphatase activity, a further anti-angiogenic compound or a compound which induces cell differentiation processes;

6. monoclonal antibodies;

7. a cyclooxygenase inhibitor, a bisphosphonate, a heparanase inhibitor, a biological response modifier;

8. an inhibitor of Ras oncogenic isoforms;

9. a telomerase inhibitor;

10. a protease inhibitor, a matrix metalloproteinase inhibitor, a methionine aminopeptidase inhibitor, or a proteasome inhibitor;

11. agents used in the treatment of hematologic malignancies or compounds which target, decrease or inhibit the activity of Flt-3;

12. an HSP90 inhibitor;

13. antiproliferative antibodies;

14. a histone deacetylase (HDAC) inhibitor;

15. a compound which targets, decreases or inhibits the activity/function of serine/threonine mTOR kinase;

16. a somatostatin receptor antagonist;

17. an anti-leukemic compound;

18. tumor cell damaging approaches;

19. an EDG binder;

20. a ribonucleotide reductase inhibitor;

21. an S-adenosylmethionine decarboxylase inhibitor;

22. a monoclonal antibody of VEGF or VEGFR;

23. photodynamic therapy;

24. an angiostatic steroid;

25. an implant containing corticosteroids;

26. an AT1 receptor antagonist;

27. an ACE inhibitor;

28. an antidiabetic agent;

29. a hypolipidemic agent;

30. an anti-obesity agent;

31. an anti-hypertensive agent; and

32. an agonist of peroxisome proliferators-activator receptors (PPARs).

A more detailed definition of these terms is found in WO2007/084344 to Shulok et al.

Specific combinations between the antibodies of the invention and the following therapeutics are contemplated. A contemplated combination partner for the treatment of cancer is an anti-estrogen including, but not limited to, tamoxifen, fulvestrant, raloxifene and raloxifene hydrochloride. A contemplated combination partner for the treatment of cancer is a protein-tyrosine kinase, such as imatinib mesylate (GLEEVEC); tyrphostin or pyrymidylaminobenzamide and derivatives thereof (AMN107). A contemplated combination partner for the treatment of a cancer or proliferative disease is one or more monoclonal antibodies including, but not limited to bevacizumab, cetuximab, trastuzumab, Ibritumomab tiuxetan, denosumab, anti-CD40, anti-GM-CSF, and tositumomab. A contemplated combination partner for the treatment of cancer or a bone related disease is a bisphosphonate including, but not limited to, etridonic, clodronic, tiludronic, pamidronic, alendronic, ibandronic, risedronic and zoledronic acid. A contemplated combination partner for the treatment of diabetes is one or more anti-diabetic agents including, but not limited to, insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as vildagliptin; thiazoladinediones. A contemplated combination partner for the treatment of obesity is one or more anti-obesity agents including, but not limited to orlistat or rimonabant, sibutramine, or phentermine.

Other pharmaceutically active agents include, but are not limited to, plant alkaloids, hormonal agents and antagonists, biological response modifiers (e.g., lymphokines or interferons), antisense oligonucleotides or oligonucleotide derivatives including silencing RNAs (siRNAs); or miscellaneous agents or agents with other or unknown mechanism of action.

In each case where citations of patent applications or scientific publications are given, in particular with regard to the respective compound claims and the final products of the working examples therein, the subject matter of the final products, the pharmaceutical preparations and the claims is hereby incorporated into the present application by reference to these publications. Comprised are likewise the corresponding stereoisomers, as well as the corresponding crystal modifications, e.g., solvates and polymorphs, which are disclosed therein. The compounds used as active ingredients in the combinations disclosed herein can be prepared and administered as described in the cited documents, respectively.

The structure of the active agents identified by code numbers, generic or trade names may be taken from the actual edition of the standard compendium “The Merck Index” or from databases, e.g., Patents International, e.g., IMS World Publications, or the publications mentioned above and below. The corresponding content thereof is hereby incorporated by reference.

It will be understood that references to the components (a) and (b) are meant to also include the pharmaceutically acceptable salts of any of the active substances. If active substances comprised by components (a) and/or (b) have, for example, at least one basic center, they can form acid addition salts. Corresponding acid addition salts can also be formed having, if desired, an additionally present basic center. Active substances having an acid group, e.g., COOH, can form salts with bases. The active substances comprised in components (a) and/or (b) or a pharmaceutically acceptable salts thereof may also be used in form of a hydrate or include other solvents used for crystallization.

Thus, in a first aspect, the present invention relates to a method for the prevention or treatment of DDK1- and/or DKK4-(DKK1/4-)associated diseases, disorders or conditions in a mammal, preferably a human patient, which comprises treating the patient concurrently or sequentially with pharmaceutically effective amounts of a combination of:

(a) a neutralizing anti-DKK1/4 composition; and

(b) an pharmaceutically active agent.

In one embodiment, the present invention provides a preparation comprising:

(a) a neutralizing anti-DKK1/4 composition; and

(b) one or more pharmaceutically active agents selected from the group consisting of an aromatase inhibitor; an antiestrogen; an anti-androgen; a gonadorelin agonist; a topoisomerase I inhibitor; a topoisomerase II inhibitor; a microtubule active agent; an alkylating agent; an anti-neoplastic anti-metabolite; a platin compound; a compound targeting/decreasing a protein or lipid kinase activity or a protein or lipid phosphatase activity, a anti-angiogenic compound; a compound which induces cell differentiation processes; monoclonal antibodies; a cyclooxygenase inhibitor; a bisphosphonate; a heparanase inhibitor; a biological response modifier; an inhibitor of Ras oncogenic isoforms; a telomerase inhibitor; a protease inhibitor, a matrix metalloproteinase inhibitor, a methionine aminopeptidase inhibitor; a proteasome inhibitor; agents which target, decrease or inhibit the activity of Flt-3; an HSP90 inhibitor; antiproliferative antibodies; an HDAC inhibitor; a compound which targets, decreases or inhibits the activity/function of serine/theronine mTOR kinase; a somatostatin receptor antagonist; an anti-leukemic compound; tumor cell damaging approaches; an EDG binder; a ribonucleotide reductase inhibitor; an S-adenosylmethionine decarboxylase inhibitor; a monoclonal antibody of VEGF or VEGFR; photodynamic therapy; an Angiostatic steroid; an implant containing corticosteroids; an AT1 receptor antagonist; and an ACE inhibitor, a bone anabolic, a weight loss therapy, an antidiabetic agent, a hypylipidemic agent, and anti-obesity agent, an anti-hypertensive agent, and/or an agonist of peroxisome proliferators-activator receptors (PPARs), or other pharmaceutically active agent provided herein.

Any of the combination of components (a) and (b), the method of treating a warm-blooded animal comprising administering these two components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as combination of the invention (so that this term refers to each of these embodiments which thus can replace this term where appropriate).

Simultaneous administration may, e.g., take place in the form of one fixed combination with two or more active ingredients, or by simultaneously administering two or more active ingredients that are formulated independently. In one embodiment, sequential use (administration) means administration of one (or more) components of a combination at one time point, other components at a different time point. In one embodiment, the combination shows more efficiency than the single compounds administered independently (especially showing synergism). In one embodiment, separate use (administration) means administration of the components of the combination independently of each other at different time points. In one embodiment, separate use means the components (a) and (b) are administered such that no overlap of measurable blood levels of both compounds are present in an overlapping manner (at the same time).

In one embodiment, combinations of two or more of sequential, separate and simultaneous administration are possible. In one embodiment the combination component-drugs show a joint therapeutic effect that exceeds the effect found when the combination component-drugs are used independently at time intervals so large that no mutual effect on their therapeutic efficiency can be found. In one embodiment a synergistic effect occurs.

The term “delay of progression” as used herein means administration of the combination to patients being in a pre-stage or in an early phase, of the first manifestation or a relapse of the disease to be treated, in which patients, e.g., a pre-form of the corresponding disease is diagnosed or which patients are in a condition, e.g., during a medical treatment or a condition resulting from an accident, under which it is likely that a corresponding disease will develop.

“Jointly therapeutically active” or “joint therapeutic effect” means that the compounds may be given separately (in a chronically staggered manner, e.g., a sequence-specific manner) in such time intervals that the subject being treated still shows an interaction (joint therapeutic effect). In one embodiment the treatment subject is a warm-blooded animal, especially human, In one embodiment, the observed interaction is synergistic. That this is the case can inter alia be determined by following the blood levels, showing that both compounds are present in the blood of the human to be treated at least during certain time intervals.

“Pharmaceutically effective” in one embodiment relates to an amount that is therapeutically or also prophylactically effective against the progression of a proliferative disease.

The term “a commercial package” or “a product”, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous or sequential (chronically staggered, in time-specific sequence, or separate) use thereof in the delay of progression or treatment of a proliferative disease. The parts of the kit of parts can then, e.g., be administered simultaneously or chronologically staggered, that is at different time points and with equal or different time intervals for any part. In one embodiment, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. In one embodiment, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in an otherwise non-effective dosage of one or both of the combination partners (components) (a) and (b). In one embodiment there is a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-tumor effect) or the like.

The combination of the invention can also be applied in combination with other treatments, e.g., surgical intervention, hyperthermia and/or irradiation therapy.

The pharmaceutical compositions according to the present invention can be prepared by conventional means and are those suitable for enteral, such as oral or rectal, and parenteral administration to mammals including man, comprising a therapeutically effective amount of a VEGF inhibitor and at least one pharmaceutically active agent alone or in combination with one or more pharmaceutically acceptable carriers, especially those suitable for enteral or parenteral application.

The pharmaceutical compositions comprise from about 0.00002 to about 100%, especially, e.g., in the case of infusion dilutions that are ready for use, of 0.0001 to 0.02%, or, e.g., in case of injection or infusion concentrates or especially parenteral formulations, from about 0.1% to about 95%, or from about 1% to about 90%, or from about 20% to about 60%; at least about any of 0.0001, 0.001, 0.01, 0.1, 1, 2.5, 5, 10, 15, 20, 25, 30, 40, 45, 50, 55, 60, 65, 70, 75, 80, 85, 90, or 95%, and/or no more than about any of 0.0001, 0.001, 0.01, 0.1, 1, 2.5, 5, 10, 15, 20, 25, 30, 40, 45, 50, 55, 60, 65, 70, 75, 80, 85, 90, or 95%, active ingredient (weight by weight, in each case). Pharmaceutical compositions according to the invention may be, e.g., in unit dose form, such as in the form of ampoules, vials, dragées, tablets, infusion bags or capsules.

The effective dosage of each combination partner in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

In one embodiment Tyrphostins, especially Adaphostin, are administered to a warm-blooded animal, especially a human in a dosage in the range of about 1-6000 mg/day, more or 25-5000 mg/day, or 50-4000 mg/day. In one embodiment, unless stated otherwise herein, the compound is administered from 1 to 5, especially from 1-4 times per day.

Pharmaceutical preparations for the combination therapy for enteral or parenteral administration are, e.g., those in unit dosage forms, such as sugar-coated tablets, capsules, suppositories, and ampoules. If not indicated otherwise, these formulations are prepared by conventional means, e.g., by means of conventional mixing, granulating, sugar-coating, dissolving or lyophilizing processes. The unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components.

In one embodiment, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed such as water, glycols, oils, alcohols, flavoring agents, preservatives, coloring agents. Pharmaceutically acceptable carriers include starches, sugars, microcrystalline celluloses, diluents, granulating agents, lubricants, binders, disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The isotonic agent may be selected from any known in the art, e.g. mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously such as antioxidants.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### EXAMPLES

### Example 1

### Generation of Human DKK1-Specific Antibodies from the HuCAL GOLD® Library

Therapeutic antibodies against human DKK1 protein are generated by selection of clones having high binding affinities, using as the source of antibody variant proteins a commercially available phage display library, the MorphoSys HuCAL GOLD® library. HuCAL GOLD® is a Fab library (Knappik et al., 2000 J. Mol. Biol. 296:57-86; Krebs et al., 2001 J Immunol. Methods 254:67-84; Rauchenberger et al., 2003 J Biol Chem. 278(40):38194-38205), in which all six CDRs are diversified by appropriate mutation, and which employs the CysDisplay™ technology for linking Fab fragments to the phage surface (WO 01/05950 Löhning 2001).

### General Procedures: Phagemid Rescue, Phage Amplification, and Purification

The HuCAL GOLD® library is amplified in standard rich bacterial medium (2×YT) containing 34 μg/ml chloramphenicol and 1% glucose (2×YT-CG). After infection of cells at an OD₆₀₀ₙₘ of 0.5 with VCSM13 helper phages (incubating the mix of cells and phage for 30 min at 37° C. without shaking followed by 30 min at 37° C. shaking at 250 rpm), cells are centrifuged (4120 g; 5 min; 4° C.), resuspended in 2×YT/34 μg/ml chloramphenicol/50 μg/ml kanamycin/0.25 mM IPTG, and grown overnight at 22° C. At the end of this period, cells are removed by centrifugation, and phages are PEG-precipitated twice from the supernatant, resuspended in PBS/20% glycerol and are stored at −80° C.

Phage amplification between two panning rounds is conducted as follows: mid-log phase E. coli strain TG1 cells infected with phages are eluted following the selection with DKK1 protein, and are plated onto LB-agar supplemented with 1% of glucose and 34 μg/ml of chloramphenicol (LB-CG plates). After overnight incubation of the plates at 30° C., bacterial colonies are scraped off the agar surface, and used to inoculate 2×YT-CG broth to obtain an OD₆₀₀ₙₘ of 0.5, then VCSM13 helper phages are added to obtain a productive infection, as described above.

### Pre-Experiments for Solution Panning Using Strep-Tactin Magnetic Beads

The Strep-tag II has been reported to have low affinity for the Strep-Tactin matrix (K_{D}˜1 μM according to (Voss and Skerra, 1997 Protein Eng. 10:975-982), therefore, a pre-experiment is performed to assess the suitability of using Strep-Tactin-coated MagStrep beads for the capturing of the antigen during the antibody selections, and to avoid antigen loss during the pannings.

For that purpose, 8 mg of MagStrep beads is incubated with 46 μg of His-Strep-tagged DKK1 for 1 h at room temperature and the sample is divided into four pre-blocked Eppendorf tubes. One tube served as the positive control (no washing) and the other three samples are washed with different stringencies according to the HuCAL GOLD® manual panning section. Detection of binding of the His-Strep-tagged DKK1 to the MagStrep beads (Strep-Tactin coated Magnetic beads obtained from IBA, Gottingen, Germany) is performed in BioVeris using a goat anti-DKK1 antibody and a Rubidium-labeled anti-goat detection antibody.

No significant loss of His-Strep-tagged DKK1 from the Strep-Tactin-coated beads is detectable when the non-washed beads are compared with those beads washed with different HuCAL® stringencies. Thus, the His-Strep-tagged DKK1 seemed to be suitable for the use in the solution pannings with Strep-Tactin-coated magnetic beads (MagStrep beads).

Selection by Panning of DKK1-Specific Antibodies from the Library

For the selection of antibodies recognizing human DKK1, two panning strategies are applied.

In summary, HuCAL GOLD® phage-antibodies are divided into four pools comprising different combinations of V_{H} master genes (pool 1 contained VH1/5λκ; pool 2 contained V_{H}3λκ; pool 3 contained V_{H}2/4/6λκ; and pool 4 contained V_{H}1-6λκ). These pools are individually subjected to two rounds of solution panning His-Strep-tagged DKK1 captured onto StrepTactin magnetic beads (Mega Strep beads; IBA), and for the third selection round only, either on His-Strep-tagged DKK1 captured onto StrepTactin magnetic beads or on APP-tagged human DKK1 protein captured by Streptavidin beads (Dynabeads® M-280 Streptavidin; Dynal) with a biotinylated anti-APP antibody.

In detail, for the solution panning using His-Strep-tagged DKK1 coupled to StrepTactin magnetic beads, the following protocol is applied: pre-blocked tubes are prepared (1.5 ml Eppendorf tubes) by treatment with 1.5 ml 2× ChemiBLOCKER diluted 1:1 with PBS over night at 4° C. Pre-blocked beads are prepared by treatment as follows: 580 μl (28 mg beads) StrepTactin magnetic beads are washed once with 580 μl PBS and resuspended in 580 μl 1× ChemiBLOCKER (diluted in one volume 1×PBS). Blocking of the beads is performed in the pre-blocked tubes over night at 4° C.

Phage particles diluted in PBS to a final volume of 500 μl for each panning condition are mixed with 500 μl 2× ChemiBLOCKER/0.1% Tween and kept for one hour at room temperature on a rotating wheel. Pre-adsorption of phage particles for removal of StrepTactin or beads-binding phages is performed twice: 160 μl of blocked StrepTactin magnetic beads (4 mg) is added to the blocked phage particles, and is incubated for 30 min at room temperature on a rotating wheel. After separation of the beads by a magnetic device (Dynal MPC-E), the phage supernatant (˜1.1 ml) is transferred to a fresh, blocked reaction tube and pre-adsorption is repeated using 160 μl blocked beads for 30 min. Then, His-Strep-tagged DKK1, either 400 nM or 100 nM, is added to the blocked phage particles in a fresh, blocked 1.5 ml reaction tube and the mixture is incubated for 60 min at room temperature on a rotating wheel.

The phage-antigen complexes are captured using either 320 μl or 160 μl of blocked StrepTactin magnetic beads added to the 400 nM or the 100 nM phage panning pools, respectively, which is then incubated for 20 min at room temperature on a rotating wheel. Phage particles bound to the StrepTactin magnetic beads are again collected with the magnetic particle separator.

Beads are then washed seven times with PBS/0.05% Tween (PB ST), followed by washing another three times with PBS only. Elution of phage particles from the StrepTactin magnetic beads is performed by addition of 200 μl 20 mM DTT in 10 mM Tris-HCl, pH 8.0 to each tube for 10 min. The eluate is collected, and the beads are washed once with 200 μl PBS and the PBS eluate is added to the DTT eluate. This eluate sample is used to infect 14 ml of an E. coli TG-1 culture that had been grown to an OD₆₀₀ₙₘ of 0.6-0.8.

After infection and subsequent centrifugation for 10 min at 5000 rpm, each bacterial pellet is resuspended in 500 μl 2×YT medium, plated onto 2×YT-CG agar plates and incubated overnight at 30° C. The next morning, the resulting colonies are scraped off the plates and the phage is prepared by rescue and amplification as described above.

The second round of solution pannings on His-Strep-tagged DKK1 is performed according to the protocol of the first round, except that decreasing amounts of antigen are used (50 nM, and 10 nM) and the stringency of the washing procedure is altered appropriately.

Two different panning strategies are applied for the third selection round: the amplified phage output of the second panning round is split and subjected to two different panning conditions. The first half of the phage output is used for the standard panning strategy on human His-Strep-tagged DKK1 captured onto StrepTactin beads as described above (antigen amounts are 10 nM or 1 nM, respectively).

The second panning variation for the third selection round is performed on human APP-tagged DKK1. APP-tagged DKK1 protein at a final concentration of 50 nM or 10 nM is mixed with 1 ml of pre-cleared, second round phage particles, and the mixture is incubated at room temperature for 1 hour on a rotating wheel. In parallel, 8 mg pre-blocked Dynabeads M-280 Streptavidin (Dynal) is incubated with 40 μg biotinylated mouse anti-APP antibody for 30 min at room temperature on a rotating wheel followed by two washing steps with PB ST. The pre-formed complexes consisting of phage-antibodies bound to APP-tagged DKK1 are captured by the anti-APP coated M-280 Streptavidin magnetic beads for 30 min at room temperature. Phage elution and amplification are performed as described above.

### Subcloning and Expression of Soluble Fab Fragments

The Fab-encoding inserts of the selected HuCAL GOLD® phagemids are subcloned into expression vector pMORPH®X9_Fab_FH, in order to facilitate rapid and efficient expression of soluble Fabs. For this purpose, the plasmid DNA of the selected clones is digested with restriction enzyme endonucleases XbaI and EcoRI, thereby excising the Fab-encoding insert (ompA-VLCL and phoA-Fd). This insert is then cloned into XbaI/EcoRI-digested expression vector pMORPH®X9_Fab_FH.

Fab proteins are expressed from this vector, and as a result carry two C-terminal tags (FLAG™ and 6× His, respectively) for both detection and purification.

Microexpression of HuCAL GOLD® Fab Antibodies in E. coli

To obtain sufficient amounts of protein encoded by each of the clones obtained above, chloramphenicol-resistant single bacterial colonies are selected after subcloning of the selected Fabs into the pMORPH®X9_Fab_FH expression vector. Each of these colonies is then used to inoculate the wells of a sterile 96-well microtiter plate, each well containing 100 μl 2×YT-CG medium per well, and bacteria are grown overnight at 37° C. A sample (50 of each E. coli TG-1 culture is transferred to a fresh, sterile 96-well microtiter plate pre-filled with 100 μl 2×YT medium supplemented with 34 μg/ml chloramphenicol and 0.1% glucose per well. The microtiter plates are incubated at 30° C. with shaking at 400 rpm on a microplate shaker until the cultures are slightly turbid (˜2-4 hrs) with an OD₆₀₀ₙₘ of about 0.5.

For expression in the format of these plates, 20 μl 2×YT medium supplemented with 34 μg/ml chloramphenicol and 3 mM IPTG (isopropyl-β-D-thiogalactopyranoside) is added per well (final concentration 0.5 mM IPTG), the microtiter plates sealed with a gas-permeable tape, and incubated overnight at 30° C. shaking at 400 rpm.

### Generation of Whole Cell Lysates (BEL Extracts)

To each well of the expression plates, 40 μl BEL buffer (2×BBS/EDTA: 24.7 g/l boric acid, 18.7 g NaCl/l, 1.49 g EDTA/l, pH 8.0) containing 2.5 mg/ml lysozyme is added, and plates are incubated for 1 h at 22° C. on a microtiter plate shaker (400 rpm). The BEL extracts are used for binding analysis by FMAT (see Example 2).

Expression of Microgram Amounts of HuCAL GOLD® Fab Antibodies in E. coli and Purification

Expression of Fab fragments encoded by pMORPH®X9_Fab_FH in E. coli TG1 F-cells is carried out in 50 ml plastic tubes. For this purpose, pre-cultures inoculated with single clones are grown in 2×YT-CG medium overnight at 30° C. The next morning, 50 μl of each pre-culture are used to inoculate 25 ml 2×YT medium supplemented with 34 μg/ml Chloramphenicol, 1 mM IPTG, and 0.1% glucose in sterile 50 ml plastic tubes, and incubated over night at 30° C. E. coli cells are harvested, the cell pellets frozen and finally disrupted with Bug Buster (Novagen). The Fab fragments are isolated using Ni-NTA Agarose (Qiagen, Hilden, Germany).

Expression of Milligram Amounts of HuCAL GOLD® Fab Antibodies in E. coli and Purification

Expression of Fab fragments encoded by pMORPH®X9_Fab_FH in TG1 F-cells is carried out in shaker flask cultures using 750 ml of 2×YT medium supplemented with 34 μg/ml chloramphenicol. Cultures are shaken at 30° C. until the OD₆₀₀ₙₘ reached 0.5. Expression is induced by addition of 0.75 mM IPTG followed by incubation for 20 h at 30° C. Cells are disrupted using lysozyme, and Fab fragments are isolated by Ni-NTA chromatography (Qiagen, Hilden, Germany). Protein concentrations are determined by UV-spectrophotometry (Krebs et al., 2001).

### Example 2

### Identification of DKK1-Specific HuCAL® Antibodies

BEL extracts of individual E. coli clones selected by the above mentioned panning strategies are analyzed by Fluorometric Microvolume Assay Technology (FMAT™, 8200 Cellular Detection System analyzer, Applied Biosystems, Foster City, Calif.), to identify clones encoding DKK1-specific Fabs. The FMAT™ 8100 HTS System is a fluorescence macro-confocal, high-throughput screening instrument that automates detection of mix-and-read, non-radioactive assays with live cells or beads (Miraglia, J. Biomol. Screening (1999), 4(4) 193-204).

Fluorometric Microvolume Assay Technology-Based Binding Analysis (FMAT) for Detection of DKK1-Binding Fabs from Bacterial Lysates

For the detection of DKK1-binding Fab antibodies from E. coli lysates (BEL extracts), binding is analyzed with the FMAT 8200 cellular detection system (Applied Biosystems). To couple His-Strep-tagged DKK1 onto M-450 Expoxy beads (Dynal), a sample of 300 μl M-450 Epoxy beads (1.2×10⁸ beads) is transferred into a reaction tube and captured with a magnetic particle separator. The supernatant is removed and the beads are washed four times in 1 ml of 100 mM sodium phosphate buffer, pH 7.4. For antigen coating, 60 μg His-Strep-tagged DKK1 is added to the bead suspension in 150 μl 100 mM sodium phosphate buffer, pH 7.4. The antigen-bead suspension is incubated for 16 h at room temperature on a rotating wheel. The coated beads are then washed three times with PBS and resuspended in a final volume of 250 μl PBS.

For each 384-well plate, a mixture of 20 ml PBS containing 3% BSA, 0.005% Tween-20, 4 μl DKK1-coated beads (1.9×10⁶ beads) and 4 μl Cy5™ detection antibody is prepared. A sample of 45 μl of this solution is dispensed per well into a 384-well FMAT black/clear bottom plate (Applied Biosystems). Fab-containing BEL extract (5 μl) is added to each well. The FMAT plates are incubated at room temperature overnight. The next morning the plates are analyzed in the 8200 Cellular Detection System (Applied Biosystems).

Positive clones are obtained, and the heavy and light chain sequences of clones yielding positive, specific signals in FMAT are analyzed. It is observed that, 57 unique (non-redundant) anti-DKK1 clones are identified that showed sufficient strong binding to human DKK1. These clones are expressed, purified and tested for affinity and in functional assays.

### Determination of Nanomolar Affinities Using Surface Plasmon Resonance

Using these clones, kinetic SPR analysis is performed on a CM5 chip (Biacore, Sweden) which had been coated with a density of ˜400 RU of either recombinant human DKK1, mouse DKK1 (R&D system), or cynomolgus DKK1 in 10 mM Na-acetate pH 4.5 using standard EDC-NHS amine coupling chemistry. A comparable amount of human serum albumin (HSA) is immobilized on the reference flow cell. PBS (136 mM NaCl, 2.7 mM KCl, 10 mM Na2HPO4, 1.76 mM KH2PO4 pH 7.4) is used as the running buffer. The Fab preparations are applied in concentration series of 16-500 nM at a flow rate of 20 μl/min. Association phase is set to 60 s and dissociation phase to 120 s. A summary of the affinities in nM to each of human, mouse, and cynomolgus DKK1 determined by that method are shown in Table 1 herein.

TABLE 1
Affinities of selected Fabs to each of human, mouse, and cynomolgus

| | KD [nM] | KD [nM] | KD [nM] |
|---|---|---|---|
| | human | mouse | cyno |
| Antibody | DKK1 | DKK1* | DKK1* |
| MOR04470 | 3.2 ± 2.0 | 3.6 | 1.7 |
| MOR04516 | 2.6 ± 0.7 | 2.4 | 1.9 |
| MOR04454 | 3.2 ± 0.4 | 6 | 2.7 |
| MOR04456 | 7.9 ± 0.9 | 11.6 | 8.1 |
| MOR04461 | 7.6 ± 3.3 | 12.8 | 7.3 |
| MOR04455 | 1.6 ± 0.3 | n.d. | 1.5 |

*single measurement
n.d.: not determined

### Example 3

### Identification of Anti-Human DKK1 Fab Candidates Inhibiting the Wnt Antagonistic Activity of DKK1

The resulting 57 different DKK1-specific antibodies selected from the HuCAL GOLD® library are used to obtain purified antibody, which is then tested for potency to inhibit the Wnt antagonistic activity of human DKK1. Of these, 17 antibody candidates are functionally active.

The functional activity of each of the HuCAL® Fabs is checked using a luciferase reporter gene assay. Twelve TCF/Lef binding sites are cloned upstream of the luciferase reporter gene rendering the luciferase gene TCF/Lef-responsive. The canonical Wnt proteins lead to a stabilization of beta-catenin, thereby activating transcription of TCF/Lef and producing the luciferase protein. Addition of DKK1 protein blocks Wnt activity and therefore also the transcription of the luciferase gene. In consequence, the luciferase levels produced by the respective cells are expected to correlate with the potency of the selected Fabs to block DKK1 action.

### Stable TCF/Lef-Responsive Reporter Cell Line HEK293T/17-12×STF

Bioassays are performed using the stable human embryonic kidney cell reporter cell line HEK293T/17-12×STF. The cells are cultivated in DMEM high glucose medium (Invitrogen), containing 10% FCS (PAN or BioWhittaker) and 1 μg/ml puromycin (BD Biosciences), until 90% confluency is reached. The cells are then trypsinized, counted, and diluted in culture medium without puromycin to a concentration of 4×10⁵ cells per ml. Subsequently, the cells are seeded into a white, flat-bottom 96-well plate (Corning; 100 μl cell suspension per well) and incubated at 37° C. and 5% CO₂ over night. On the next day, the assay medium is prepared: 500 ng/ml DKK1-APP is added to Wnt3a Conditioned Medium (CM). The anti-DKK1 HuCAL® Fabs (final concentration 20 μg/ml) and the goat anti-human DKK1 antibody (R&D Systems) used as a positive control (final concentration 1.5 μg/ml) are diluted in CM.

A volume of 60 μl medium is removed from each well of the assay plate without disturbing the adhering cells, and substituted by 60 μl of the test antibody or control, diluted in CM. The cells are incubated for another 24 h and 100 μl Bright-Glo luciferase reagent is added to each well. After 5 min incubation time, the luminescence is read in a luminometer (GenioPro, Tecan). The extent of luciferase expressed is a measure of the extent of antibody present.

### Example 4

### Quantitative Analysis of Binding Affinities: Determination of Anti-Human DKK1 Fab Candidates that Inhibit the Wnt-Antagonistic Activity of DKK1

### Affinity Determination

In order to further characterize the anti-DKK1 antibodies, the affinity to human, cynomolgus, and mouse DKK1 is determined. The recombinant DKK1 protein is immobilized on a CM5 Biacore chip and the Fabs are applied in the mobile phase in different concentrations. For a reliable determination of monovalent affinities only such Fab batches are used for Biacore measurements which showed ≧90% monomeric fraction in a size exclusion chromatography.

The summarized affinity data on human, mouse, and cynomolgus DKK1 is shown in Table 2. All 17 tested Fabs are found to have affinity to human DKK1 below 100 nM. Further, nine of the clones produced antibodies with affinities less than 10 nM. In all tested cases, the affinities for cynomolgus and mouse DKK1 are almost identical to those for human DKK1.

TABLE 2
Affinity data of selected Fabs on human, mouse, and cynomolgus

| | KD [nM] | KD [nM] | |
|---|---|---|---|
| | human | mouse | KD [nM] cyno |
| Antibody | DKK1 | DKK1* | DKK1* |
| MOR04455 | 1.6 ± 0.3 | n.d. | 1.5 |
| MOR04516 | 2.6 ± 0.7 | 2.4 | 1.9 |
| MOR04470 | 3.2 ± 2.0 | 3.6 | 1.7 |
| MOR04454 | 3.2 ± 0.4 | 6 | 2.7 |
| MOR04461 | 7.6 ± 3.3 | 12.8 | 7.3 |
| MOR04456 | 7.9 ± 0.9 | 11.6 | 8.1 |

*single measurement
n.d.: not determined

### EC₅₀ Determination

The data showing the effective concentration for 50% inhibition for the clones of antibodies having the greatest affinity for DKK1 is shown in Table 3 herein. The data show that effective concentrations EC₅₀ range from 39-95 nM, with a median value between 58 and 83 nM.

TABLE 3
Effective concentration for 50% inhibition of selected Fabs

| | Luciferase |
|---|---|
| | reporter assay; |
| Antibody | EC₅₀ [nM] |
| MOR04470 | 58 |
| MOR04516 | 42 |
| MOR04454 | 83 |
| MOR04456 | 95 |
| MOR04461 | 57 |
| MOR04455 | 39 |

### Example 5

### Affinity Maturation of Selected Anti-DKK1 Fabs by Parallel Exchange of LCDR3 and HCDR2 Cassettes

For optimizing the affinities of the antibodies described herein for DKK1 for a pool of parental Fab fragments, the LCDR3, framework 4 and the constant region of the light chains (405 bp) of each parental Fab is removed using BpiI and SphI, and is replaced by a repertoire of diversified LCDR3s together with framework 4 and the constant domain. A sample of 0.5 μg of the binder pool vector is ligated with a 3-fold molar excess of the insert fragment carrying the diversified LCDR3s.

In a similar approach, the HCDR2 is diversified using the XhoI and BssHII sites, and the connecting framework regions are kept constant. In order to increase the cloning efficiency, the parental HCDR2 is replaced by a 590 bp stuffer sequence prior to the insertion of the diversified HCDR2 cassette.

Ligation mixtures of 11 different libraries are electroporated into 4 ml E. coli TOP10 F′ cells (Invitrogen, Carlsbad, Calif., USA), yielding from 2×10⁷ to 2×10⁸ independent colonies. Amplification of the libraries is performed as previously described (Rauchenberger et al., 2003 J Biol Chem. 278(40):38194-38205). For quality control, several clones per library are randomly picked and sequenced (SequiServe, Vaterstetten, Del.) using primers CFR84 (VL) and OCAL_Seq_Hp (VH).

### Selection of Candidates for Affinity Maturation

Six selected maturation candidates (“parental Fabs”) are selected by having been characterized as having the following properties: affinities to human DKK1 less than 10 nM, with significant cross-reactivity to cynomolgus and mouse DKK1, EC₅₀ less than 100 nM, and good to moderate Fab expression levels in E. coli and lack of aggregation after Fab purification.

During the course of the affinity measurements, it became evident that MOR04480 is highly unstable at high dilutions. For this reason, MOR04480 is omitted from the list of maturation candidates albeit having the highest affinity (1 nM) and the best EC₅₀ (7 nM) of all tested Fabs. MOR04483 had a high affinity of 5.5 nM to human DKK1 but is shown to be crossreactive to mouse DKK1, and MOR04453 contained a high proportion of Fab aggregates after purification. Therefore, these two antibodies are also excluded from the maturation.

After careful evaluation of all available data, six maturation candidates (MOR04454, MOR04455, MOR04456, MOR04461, MOR04470, and MOR04516) are selected. The properties of these candidates are listed in Table 4 herein.

TABLE 4
Properties of selected Fabs

| | KD [nM] | KD [nM] | KD [nM] | | Cross | Fab | Size |
|---|---|---|---|---|---|---|---|
| | human | mouse | cyno | EC50 | reactivity | expression | exclusion |
| Antibody | DKK1 | DKK1* | DKK1* | [nM] | mouse | [mg/l] | chromatography |
| MOR04470 | 3.2 ± 2.0 | 3.6 | 1.7 | 58 | ++ | 32.8 | # |
| MOR04516 | 2.6 ± 0.7 | 2.4 | 1.9 | 42 | ++ | 1.5 | # |
| MOR04454 | 3.2 ± 0.4 | 6 | 2.7 | 83 | ++ | 17.8 | # |
| MOR04456 | 7.9 ± 0.9 | 11.6 | 8.1 | 95 | ++ | 10.7 | # |
| MOR04461 | 7.6 ± 3.3 | 12.8 | 7.3 | 57 | ++ | 12 | # |
| MOR04455 | 1.6 ± 0.3 | n.d. | 1.5 | 39 | ++ | 9 | # |

*single measurement
n.d.: not determined
# monomeric portion > 90%

### Generation of Selected Fab Libraries for Affinity Maturation

In order to obtain clones having increased affinity and inhibitory activity of the anti-DKK1 antibodies, the selected Fab clones MOR04454, MOR04455, MOR04456, MOR04461, MOR04470, and MOR4516 shown in the previous example are subjected to further rounds of diversification and selection, a process known as affinity maturation.

For this purpose, CDR regions are diversified using corresponding LCDR3 and HCDR2 maturation cassettes pre-built by trinucleotide mutagenesis (Virnekäs et al., 1994 Nucleic Acids Res. 22:5600-5607; Nagy et al., 2002 Nature Medicine 8:801-807). Table 5 herein shows the LCDR3 sequences for the parental clones MOR04454, MOR04455, MOR04456, MOR061, MOR04470 and MOR4516.

TABLE 5
LCDR3 sequences for selected Fabs

| | | | Variable | |
|---|---|---|---|---|
| | | LCDR3 | region of | |
| Antibody | VL | Sequence | SEQ ID NO: | SEQ ID NO: |
| MOR04454 | K1 | LQYYGMPP | 21 | 80 |
| MOR04455 | K1 | QQYDSIPM | 22 | 81 |
| MOR04456 | K3 | QQYGDEPI | 23 | 82 |
| MOR04470 | L2 | QSYASGNTKV | 25 | 92 |
| MOR04461 | L2 | STWDMTVDF | 24 | 87 |
| MOR04516 | L1 | ASFDMGSPNV | 26 | 98 |

Table 6 herein shows the HCDR3 sequences for the parental clones MOR04454, MOR04455, MOR04456, MOR061, MOR04470 and MOR4516.

TABLE 6
H-CDR3 sequences for selected Fabs

| | | | Variable | |
|---|---|---|---|---|
| | | | region of | |
| Antibody | VH | H-CDR3 Sequence | SEQ ID NO: | SEQ ID NO: |
| MOR04454 | H3 | DGSHMDKPPGYVFAF | 2 | 69 |
| MOR04455 | H3 | HYMDH | 3 | 64 |
| MOR04456 | H3 | TIYMDY | 4 | 65 |
| MOR04461 | H3 | MGIDLDY | 5 | 66 |
| MOR04470 | H3 | HGIDFDH | 6 | 67 |
| MOR04516 | H5 | GIPFRMRGFDY | 7 | 68 |

Fab fragments from expression vector pMORPH®X9_Fab_FH are subcloned into the phagemid vector pMORPH®25 (see U.S. Pat. No. 6,753,136). This vector provides the phage protein pIII fused N-terminally to a cysteine residue as well as a C-terminal cysteine to the Fd antibody chain and thus allows disulfide-linked display of the respective Fab fragments on the phage surface. Two different strategies are applied in parallel to optimize both the affinity and the efficacy of the parental Fabs.

Five phage antibody Fab libraries are generated in which the LCDR3 of five of the six parental clones is replaced by a repertoire of individual light chain CDR3 sequences. The LCDR3 maturation of MOR04454 is not performed, as this clone has an additional BpiI restriction site in one of the CDR regions and the BpiI restriction enzyme is used for the library cloning procedure.)

In parallel, the HCDR2 region of each parental clone is replaced by a repertoire of individual heavy chain CDR2 sequences. Each parental Fab is excised and replaced for a 590 bp stuffer. This DNA stuffer facilitates the separation of single digested from double digested vector bands and reduces the background of the high-affinity parental Fabs during the maturation pannings. In a subsequent step, the stuffer is excised from the Fab-encoding plasmids of each parental clone and replaced for the highly diversified HCDR2 maturation cassette.

Large affinity maturation libraries of more than 2×10⁷ members are generated by standard cloning procedures, and the diversified clones are transformed into electro-competent E. coli TOP10F′ cells (Invitrogen). Fab-presenting phages are prepared as described in Example 1 above.

Four maturation pools are built in order to facilitate the subsequent selection process: pool 1a consisted of the MOR04470, and MOR04516 LCDR3 libraries; pool 1b consisted of the MOR04470, and MOR04516 HCDR2 libraries; pool 2a consisted of the MOR04454, MOR04455, MOR04456, and MOR04461 LCDR3 libraries; and pool 2b consisted of the MOR04454, MOR04455, MOR04456, and MOR04461 HCDR2 libraries.

For each pool the panning is performed in solution using decreasing amounts of His-Strep-tagged DKK1 and phage-antigen capturing by Strep-Tactin beads. In parallel, each pool is applied in pannings using decreasing amounts of biotinylated DKK1, which is captured onto Neutravidin-coated plates. In order to increase the panning stringency and to select for improved off rates, competition with purified parental Fabs as well as unlabeled antigen is performed during prolonged incubation periods.

Immediately after the pannings the enriched phagemid pools are subcloned into the pMORPH®X9_FH expression vector. About 2300 single clones are picked, and the Fabs are induced with IPTG.

### Maturation Panning Strategies

Panning procedures using the four antibody pools are performed with His-Strep-tagged DKK1 and with biotinylated His-Strep-tagged DKK1 in solution for two or three rounds, respectively. For each of the panning strategies, competition with the purified parental Fab proteins or with unlabeled APP-tagged DKK1, as well as low antigen concentrations and extensive washing, are used to increase stringency.

The solution panning on unlabeled His-Strep-tagged DKK1 is performed over two selection rounds mainly according to the standard protocol described in Example 1. Exceptions to these procedures are the application of reduced amounts of antigen (decreasing from 5 nM down to 1 nM), the high stringency of the washing procedure either with competitor or without, and prolonged incubation periods of antibody-phages together with the antigen.

For the first selection round using biotinylated DKK1, the wells of a Neutravidin plate are washed two times with 300 μl PBS. The wells are blocked with 2× ChemiBLOCKER (Chemicon, Temecula, Calif.) diluted 1:1 in PBS (Blocking Buffer). Prior to the selections, the HuCAL GOLD® phages are also blocked with one volume Blocking Buffer containing 0.1% Tween-20 for 30 min at room temperature. The blocked phage preparations are transferred in 100 μl aliquots to the wells of a Neutravidin-coated plate for 30 min at room temperature. This pre-adsorption step is repeated once. Blocked and pre-cleared phage preparations are incubated with 5 nM biotinylated DKK1 for 2 h at 22° C. on a rotating wheel. Parental Fab, APP-DKK1 or no competitor is added and the samples are incubated overnight at 4° C. on a rotating wheel.

Antigen-phage complexes are captured in the wells of a Neutravidin plate for 20 min at room temperature. After extensive washing steps, bound phage particles are eluted by addition of 200 μl of 20 mM DTT in 10 mM Tris pH 8.0 per well for 10 min at room temperature. The eluate is removed and added to 14 ml E. coli TG1 cells grown to an OD₆₀₀ₙₘ of 0.6-0.8. The wells are rinsed once with 200 μl PBS and this solution is also added to the E. coli TG1 cells. Phage infection of E. coli is allowed for 45 min at 37° C. without shaking. After centrifugation for 10 min at 5000 rpm, the bacterial pellets are each resuspended in 500 μl 2×YT medium, plated onto 2×YT-CG agar plates and incubated overnight at 30° C. The colonies are harvested by scraping from the surface of the plates and the phage particles are rescued and amplified as described above.

The second and third round of the selection are performed as described above for the first round of selection, excepted that washing conditions are more stringent and antigen concentrations are 1 and 0.1 nM, respectively.

### Electrochemiluminescence (BioVeris)-Based Binding Analysis of DKK1 Binding Fabs

For the detection of affinity-improved, DKK1-specific antibody fragments in E. coli lysates (BEL extracts), a BioVeris M-384 SERIES® Workstation (BioVeris Europe, Witney, Oxfordshire, UK), is used. The assay is carried out in 96-well polypropylene microtiter plates and PBS supplemented with 0.5% BSA and 0.02% Tween-20 as the assay buffer. Biotinylated human DKK1 is immobilized on M-280 Streptavidin paramagnetic beads (Dynal) according to the instructions of the supplier. A 1:25 dilution of the bead stock solution is added per well. Samples of 100 μl diluted BEL extract and beads are incubated overnight at room temperature on a shaker. For detection, anti-human (Fab)′2 (Dianova) labelled with BV-tag™ according to instructions of the supplier (BioVeris Europe, Witney, Oxfordshire, UK) is used.

A set of about 2300 randomly picked clones are analyzed by the method described above. A subset of 160 clones giving the highest values is chosen for further analysis in solution equilibrium titration.

### Determination of Picomolar Affinities Using Solution Equilibrium Titration (SET)

For K_{D} determination, monomer fractions (at least 90% monomer content, analyzed by analytical SEC; Superdex75, Amersham Pharmacia) of Fab are used. Electrochemiluminescence (ECL) based affinity determination in solution and data evaluation are basically performed as described by Haenel et al., 2005. A constant amount of Fab is equilibrated with different concentrations (serial 3ⁿ dilutions) of human DKK1 (4 nM starting concentration) in solution. Biotinylated human DKK1 coupled to paramagnetic beads (M-280 Streptavidin, Dynal), and BV-tag™ (BioVeris Europe, Witney, Oxfordshire, UK) labelled anti-human (Fab)′₂ (Dianova) is added and the mixture incubated for 30 min. Subsequently, the concentration of unbound Fab is quantified by ECL detection using the M-SERIES® 384 analyzer (BioVeris Europe).

For this purpose, 160 single clones are selected and purified by Ni-NTA Agarose in them scale. Preliminary affinities are determined by 4-point solution equilibrium titration (SET) in BioVeris. From these data, 20 clones showing affinities are selected. These Fabs are purified in the mg scale. MOR04950 is excluded from affinity determination and further evaluation due to partial aggregation of the Fab which is detected in size exclusion chromatography. Final affinities are determined from two independent batches of each Fab clone using an 8-point SET measurement and human, mouse, and cynomolgus DKK1.

Affinity determination to mouse and cynomolgus DKK1 is done essentially as described above using mouse DKK1 (R&D Systems) and cynomolgus DKK1 as analyte in solution instead of human DKK1. For detection of free Fab, biotinylated human DKK1 coupled to paramagnetic beads is used. Affinities are calculated according to Haenel et al., 2005 Anal Biochem 339.1:182-184.

Using the assay conditions described above, the affinities for the affinity-optimized anti-DKK1 Fabs are determined in solution. Affinities are determined to human DKK1 and to mouse and cynomolgus DKK1.

### Example 6

### Characterization of Affinity-Optimized Anti-Human DKK1 Fabs Enzyme Linked Immuno Sorbent Assay (ELISA) Techniques

Binding specificity of the matured Fabs in the presence of 50% human serum (HS) is determined Serial dilutions of human recombinant, biotinylated DKK1 in TBS are coated onto Neutravidin microtiter plates for 2 h at room temperature, from 8 ng DKK1 per well to a concentration of 125 ng DKK1 per well. After coating of the antigen, wells are blocked with TBS/0.05% Tween (TBS-T) supplemented with 1% BSA for 1 h at room temperature. Purified Fabs described above are diluted either in TBS/4% BSA or TBS/50% HS at a final concentration of 1 μg/ml, added to the coated and blocked wells and the plates are incubated for 1 h at room temperature. For detection, an anti-FLAG alkaline phosphatase (AP)-conjugated antibody (1:5000 dilution in TBST) and the fluorogenic substrate AttoPhos (Roche) are used. After each incubation, the wells of the microtiter plates are washed with TBST five times, except after the final incubation step with the labeled secondary antibody when wells are washed three times.

The fluorescence is measured in a TECAN Spectrafluor plate reader. The binding activity of the optimized anti-DKK1 Fabs is determined in presence of 50% human serum compared to binding activity in 4% BSA. The median value is found to be 93%, thus the anti-DKK1 Fabs are found to fully bind to target in the presence of human serum.

### Luciferase Reporter Cell Assay in Presence of Human Serum Using the U2OS Cell Line

For a further determination of binding specificity of the optimized anti-DKK1 Fabs, the luciferase reporter cell assay is repeated in presence of 15% human serum using the osteosarcoma cell line U2OS. The U2OS cells (ATCC No. HTB-96) are grown according to the provider's protocol (ATCC, Manassas, Va., USA). The cells are trypsinized, counted, and diluted in culture medium (McCoy's 5a/10% FCS) to a concentration of 2×10⁵ cells/ml. For each 2×10⁴ cells, a solution is prepared that is a mixture of 0.075 μg pTA-LUC-12× SuperTopFlash and 0.004 μg phRL-SV40. These are mixed in a final volume of 9.8 μl OPTI-MEM. Then 0.2 μl FuGENE 6 Transfection Reagent (Roche, Mannheim, Germany) is added. This transfection mix is briefly incubated and then mixed with the previously prepared cells. Subsequently, the cells are seeded in 100 μl per well of a white flat-bottomed 96-well cell culture dish and incubated at 37° C. and 5% CO₂ over night. The next day, 75 μl medium are removed from each well of the assay plate and substituted by 10 μl of HuCAL® Fab antibodies dilutions from (10 to 0.01 μg/ml diluted in serum-free culture medium), 15 μl of either 70% FCS or Human Serum, and 50 μl of the Wnt3a Conditioned Medium, containing 600 ng/ml DKK1-APP is added to each well.

For a negative control, serum-free medium is added instead of antibody dilutions. In order to obtain a maximum luciferase signal, controls containing 10 μl serum-free medium instead of antibody dilutions and 50 μl Wnt3a CM without DKK1-APP are added. After 24 h incubation at 37° C., 5% CO₂, the luminescence is measured with the Dual-Glo Luciferase Assay System (Promega, Madison, Wis., USA) according to the manufacturer's instructions.

These data show that clones of the anti-DKK1 Fabs are obtained that function in the presence of human serum.

### EC₅₀ Determination of Affinity-Optimized Anti-DKK1 Fabs by Luciferase Reporter Cell Assay

The test of the affinity-improved Fabs in the standard Wnt3a-dependent TCF/LEF luc reporter assay used 10 nM DKK1 in order to obtain inhibition of the luciferase expression. It is seen that EC₅₀ values could not be generated by this method as the sensitivity of the assay is too low. This is indicated by very steep inhibition curves and similar EC₅₀ values for all Fabs tested.

An improved version of the TCF/LEF luc reporter assay is developed. DKK1 binds to the Kremen-1 and -2 transmembrane proteins and this interaction leads to a strong synergistic inhibition of Wnt signaling (Mao et al. 2002 Nature: 417: 664-67). Therefore, Kremen cDNA is co-transfected with the TCF/LEF luc reporter assay. The resulting Wnt3a-dependent reporter assay showed highly improved sensitivity to DKK1, mediated by co-expression of the Kremen co-receptor protein. In this assay, 0.33 nM DKK1 is sufficient to induce full inhibition of Wnt signaling. The Fab titrations (at ten concentrations) are repeated using 0.33 nM DKK1, and yielded sigmoid inhibition curves from which EC₅₀ values could be calculated.

The affinity-optimized anti-DKK1 Fabs are thereby analyzed with respect to EC₅₀ as described above. The EC₅₀ values obtained by this method ranged from 0.2 nM to 5.6 nM.

### Sequence Analysis of the Affinity-Optimized Fabs

The nucleotide sequences of the heavy and V_{L} regions (V_{H} and V_{L}) of all twenty Fabs are determined Amino acid sequences of the complementarity determining regions (CDRs) are listed in Table 7 and Table 8 herein

TABLE 7
Amino acid sequences of Heavy Chain CDR′s

| | | | | SEQ | | SEQ | | SEQ |
|---|---|---|---|---|---|---|---|---|
| | | | | ID No. | | ID No. | | ID No. |
| | Antibody | VH | HCDR1 | HCDR1 | HCDR2 | HCDR2 | HCDR3 | HCDR3 |
| P | MOR04455 | VH3 | GFTFSSYGMS | 49 | wvsGISGSGSYTYYADSVKG | 53 | HYMDH | 64 |
| 1 | MOR04918 | VH3 | GFTFSSYGMS | 49 | wvsGISERGVYIFYADSVKG | 54 | HYMDH | 64 |
| P | MOR04456 | VH3 | GFTFNNYGMT | 50 | wvsGISGSGSYTYYADSVKG | 53 | TIYMDY | 65 |
| 2 | MOR04907 | VH3 | GFTFNNYGMT | 50 | wvsGISGSGSYTYYADSVKG | 53 | TIYMDY | 65 |
| 3 | MOR04946 | VH3 | GFTFNNYGMT | 50 | wvsGISGSGSYTYYADSVKG | 53 | TIYMDY | 65 |
| 4 | MOR04949 | VH3 | GFTFNNYGMT | 50 | wvsGISGSGSYTYYADSVKG | 53 | TIYMDY | 65 |
| 5 | MOR04913 | VH3 | GFTFNNYGMT | 50 | wvsGISGSGSYTYYADSVKG | 53 | TIYMDY | 65 |
| P | MOR04461 | VH3 | GFTFSSYWMS | 51 | wvsGISYSGSNTHYADSVKG | 55 | MGIDLDY | 66 |
| 6 | MOR04911 | VH3 | GFTFSSYWMS | 51 | wvsDIEHKRRAGGATSYAASVKG | 56 | MGIDLDY | 66 |
| 7 | MOR04922 | VH3 | GFTFSSYWMS | 51 | wvsMIEHKTRGGTTDYAAPVKG | 57 | MGIDLDY | 66 |
| 8 | MOR04910 | VH3 | GFTFSSYWMS | 51 | wvsGISYSGSNTHYADSVKG | 55 | MGIDLDY | 66 |
| 9 | MOR04948 | VH3 | GFTFSSYWMS | 51 | wvsGISYSGSNTHYADSVKG | 55 | MGIDLDY | 66 |
| 10 | MOR04919 | VH3 | GFTFSSYWMS | 51 | wvsGISYSGSNTHYADSVKG | 55 | MGIDLDY | 66 |
| 11 | MOR04921 | VH3 | GFTFSSYWMS | 51 | wvsGISYSGSNTHYADSVKG | 55 | MGIDLDY | 66 |
| P | MOR04470 | VH3 | GFTFSSYWMS | 51 | wvsVISSDSSSTYYADSVKG | −58 | HGIDFDH | −67 |
| 12 | MOR04914 | VH3 | GFTFSSYWMS | 51 | wvsVISSDSSSTYYADSVKG | 58 | HGIDFDH | 67 |
| 13 | MOR04945 | VH3 | GFTFSSYWMS | 51 | wvsVISSDSSSTYYADSVKG | 58 | HGIDFDH | 67 |
| 14 | MOR04951 | VH3 | GFTFSSYWMS | 51 | wvsVISSDSSSTYYADSVKG | 58 | HGIDFDH | 67 |
| 15 | MOR04952 | VH3 | GFTFSSYWMS | 51 | wvsVISSDSSSTYYADSVKG | 58 | HGIDFDH | 67 |
| 16 | MOR04950 | VH3 | GFTFSSYWMS | 51 | wvsVIEHKSFGSATFYAASVKG | 59 | HGIDFDH | 67 |
| 17 | MOR04954 | VH3 | GFTFSSYWMS | 51 | wvsVIEHKDKGGITYYAASVKG | 60 | HGIDFDH | 67 |
| 18 | MOR04920 | VH3 | GFTFSSYWMS | 51 | wvsSIEHKDAGYTTWYAAGVKG | 61 | HGIDFDH | 67 |
| P | MOR04516 | VH5 | GYSFTNYYIG | 52 | wmgIIYPTDSYTNYSPSFQG | 62 | GIPFRMRGFDY | 68 |
| 19 | MOR04947 | VH5 | GYSFTNYYIG | 52 | wmgIIYPGTSYTIYSPSFGQ | 63 | GIPFRMRGFDY | 68 |
| | Consensus | | GFTFNNYGMT | 40 | GISGSGSYTYYADSVKG | 44 | X(G/X)I(D/Y) | 48 |
| | | | | | | | XD(Y/H) | |
| | Consensus | | GFTFSSYWMT | 41 | GISYSGSNTHYDSVKG | 45 | | |
| | Consensus | | GFTF(S/N)(S/N)Y | 42 | VISSDSSSTYYADSVKG | 46 | | |
| | (G/W)X(S/T/X) | | | | | | | |
| | Consensus | | GYSFTNYYIG | 43 | II(Y/V)PXXSYT(N/I) | 47 | | |
| | | | | | YSPSFQG | | | |

Consensus H-CDR sequences SEQ ID NOs: 40-48 are derived from Table 18A and provided in Table 7. Additional consensus CDR sequences for either heavy or light chains of the invention may be determined by one skilled in the art from the alignments in Tables 18A-18C using standard methods and methods provided herein.

TABLE 8
Amino acid sequences of Light Chain CDR's

| | MOR | | | SEQ | | SEQ | | SEQ |
|---|---|---|---|---|---|---|---|---|
| | Antibody | | | ID | | ID | | ID |
| | No. | VL | HCDR1 | No. | HCDR2 | No. | hCDR3 | No. |
| P | 04455 | K1 | RASQDISNYLH | 70 | LLIYGASNLQS | 75 | QQYDSIPM | 81 |
| 1 | 04918 | K1 | RASQDISNYLH | 70 | LLIYGASNLQS | 75 | QQYDSIPM | 81 |
| P | 04456 | K3 | RASQNLFSPYLA | 71 | LLIYGASNRAT | 76 | QQYGDEPI | 82 |
| 2 | 04907 | K3 | RASQNLFSPYLA | 71 | LLIYGASNRAT | 76 | QQYLSLPT | 83 |
| 3 | 04946 | K3 | RASQNLFSPYLA | 71 | LLIYGASNRAT | 76 | QQYLTLPL | 84 |
| 4 | 04949 | K3 | RASQNLFSPYLA | 71 | LLIYGASNRAT | 76 | QQYLFPL | 85 |
| 5 | 04913 | K3 | RASQNLFSPYLA | 71 | LLIYGASNRAT | 76 | QQYMTLPL | 86 |
| P | 04461 | L2 | TGTSSDVGGFNYVS | 72 | LMIHDGSNRPS | 77 | STWDMTVDF | 87 |
| 6 | 04911 | L2 | TGTSSDVGGFNYVS | 72 | LMIHDGSNRPS | 77 | STWDMTVDF | 87 |
| 7 | 04922 | L2 | TGTSSDVGGFNYVS | 72 | LMIHDGSNRPS | 77 | STWDMTVDF | 87 |
| 8 | 04910 | L2 | TGTSSDVGGFNYVS | 72 | LMIHDGSNRPS | 77 | QSWDVSPITA | 88 |
| 9 | 04948 | L2 | TGTSSDVGGFNYVS | 72 | LMIHDGSNRPS | 77 | QTWDSLSFF | 89 |
| 10 | 04919 | L2 | TGTSSDVGGFNYVS | 72 | LMIHDGSNRPS | 77 | QSWGVGPGGF | 90 |
| 11 | 04921 | L2 | TGTSSDVGGFNYVS | 72 | LMIHDGSNRPS | 77 | QTWATSPLSS | 91 |
| P | 04470 | L2 | TGTSSDLGGYNYVS | 73 | LMIYDVNNRPS | 78 | QSYASGNTKV | 92 |
| 12 | 04914 | L2 | TGTSSDLGGYNYVS | 73 | LMIYDVNNRPS | 78 | QSYTYTPISP | 93 |
| 13 | 04945 | L2 | TGTSSDLGGYNYVS | 73 | LMIYDVNNRPS | 78 | QTYDQIKLSA | 94 |
| 14 | 04951 | L2 | TGTSSDLGGYNYVS | 73 | LMIYDVNNRPS | 78 | QSYDPFLDW | 95 |
| 15 | 04952 | L2 | TGTSSDLGGYNYVS | 73 | LMIYDVNNRPS | 78 | QSYDSPTDSV | 96 |
| 16 | 04950 | L2 | TGTSSDLGGYNYVS | 73 | LMIYDVNNRPS | 78 | QSYASGNTKV | 97 |
| 17 | 04954 | L2 | TGTSSDLGGYNYVS | 73 | LMIYDVNNRPS | 78 | QSYASGNTKV | 97 |
| 18 | 04920 | L2 | TGTSSDLGGYNYVS | 73 | LMIYDVNNRPS | 78 | QSYASGNTKV | 97 |
| P | 04516 | L1 | SGSSSNIGSSFVN | 74 | LLIGNNSNRPS | 79 | ASFDMGSPNV | 98 |
| 19 | 04947 | L1 | SGSSSNIGSSFVN | 74 | LLIGNNSNRPS | 79 | ASFDMGSPNV | 98 |
| | consensus1 | | RASQxxxxxYx | 113 | LLIYGASNxxx | 114 | QQYxxxPx | 115 |
| | consensus2 | | TGTSSDVGGFNYVS | 116 | LMIxDxxNRPS | 117 | xxWDxxxxx | 118 |

The sequence analysis showed that five of the six parental (P) Fabs yielded affinity-improved successors. MOR04461 and MOR04470 could be optimized in HCDR2 as well as in LCDR3. No optimized successors of MOR04454 are obtained. In addition, high homology appears between disparate parent antibodies, as shown in consensus1 and consensus2 sequences for the various CDRs in Table 8 Similar consensus sequences may be provided for parent sequences shown in Table 10A using methods well known to one skilled in the art.

In addition, it is determined that MOR04920 has a mutation in the HCDR2 region (a Ser residue to a Gly at pos. 73 according to the numbering scheme published by Honegger and Pluckthun, 2001 J Mol Biol 309.3:657-670 thus deviating from the HuCAL® design.

MOR04913 is shown to have a point mutation in framework 4 of the kappa light chain (Lys to Asn exchange at position 148). As this position is not expected to have an effect on the binding properties of the antibody the mutation is reverted back to the germline/HuCAL® composition during IgG conversion, yielding antibody MOR05145.

MOR04947 has a potential glycosylation site in LCDR2. This site is not removed as MOR04947 is selected only as one of the back-up candidates.

### Example 7

### Production of HuCAL® Immunoglobulins

Conversion into the IgG Format

In order to express full length immunoglobulin (Ig), variable domain fragments of heavy (V_{H}) and light chains (V_{L}) are subcloned from the pMORPH®X9_FH Fab expression vectors either into the pMORPH®h_Ig or the pMORPH®2_h_Ig vector series for human IgG1 and human IgG4. Alternative vectors may be used for human IgG2. Restriction enzymes EcoRI, MfeI, and BlpI are used for subcloning of the V_{H} domain fragment into pMORPH®h_IgG1 and pMORPH®_h_IgG4. Restriction enzymes MfeI and BlpI are used for subcloning of the V_{H} domain fragment into pMORPH®2_h_IgG1f and pMORPH®2_h_IgG4. Subcloning of the V_{L} domain fragment into pMORPH®_h_Ig1c and pMORPH®2_h_Ig1c is performed using the EcoRV and BsiWI sites, whereas subcloning into pMORPH®_h_Igk and pMORPH®2_h_Ig22 is done using EcoRV and HpaI.

### Transient Expression and Purification of Human IgG

HEK293 cells are transfected with an equimolar amount of IgG heavy and light chain expression vectors. On days 4 or 5 after transfection, the cell culture supernatant is harvested. After adjusting the pH of the supernatant to 8.0 and sterile filtration, the solution is subjected to standard protein A column chromatography (Poros 20A, PE Biosystems).

Conversion of Parental Fabs into the IgG Formats

In parallel to the start of the affinity maturation, MOR04454, MOR04456, and MOR04470 are cloned into the pMORPH®h_IgG1 and pMORPH®_h_IgG4 expression vectors. Alternative constructs may be used for creation of IgG2 expression vectors. Small scale expression is performed by transient transfection of HEK293 cells and the full length immunoglobulins are purified from the cell culture supernatant.

The data show by size exclusion chromatography that the antibodies are in monomeric form. Testing in the Wnt3a-dependent reporter assay proved that the proteins are functional.

### Example 8

### Amino Acid Sequences and Nucleotide Sequences of Genes Optimized for Expression

To increase mammalian expression, changes are introduced into the heavy and the light chains of Fabs herein for optimization of codon usage for expression in a cell. It is known that several negatively cis-acting motifs decrease expression in mammals. The optimization process herein removes negative cis-acting sites (such as splice sites or poly(A) signals) which negatively influence expression. The optimization process herein further enriches GC content, to prolong mRNA half-life.

Variable light and heavy chain regions are optimized using a clone of a Fab, MOR04945 (full length light chain parental nucleotide sequence is SEQ ID NO: 98 and full length heavy chain parental nucleotide sequence is SEQ ID NO: 102), isolated herein by selection with phage display. Then the nucleotide sequences encoding each of the entire light and heavy chains of this and other clones are each optimized using these procedures.

### Optimization Process for V_{H} V_{L} Chains of MOR04945

For optimizing the nucleotide sequence and amino acid sequence of each of the V_{L} and V_{H} chains for expression in mammalian cells, the codon usage is adapted to the codon bias of mammalian genes. In addition, regions of very high (>80%) or very low (<30%) GC content are reduced or eliminated where possible. Alternatively, optimization for expression in bacteria, yeast or baculovirus would entail adapting codon usage biased for their respective genes.

During the optimization process for mammalian expression, the following cis-acting sequence motifs are avoided: internal TATA-boxes, chi-sites and ribosomal entry sites, AT-rich or GC-rich sequence stretches, RNA instability motif (ARE) sequence elements, inhibitory RNA sequence elements (INS), cAMP responsive (CRS) sequence elements, repeat sequences and RNA secondary structures, splice donor and acceptor sites including cryptic sites, and branch points. Except as indicated, introduction of MluI and HindIII sites is avoided in the process of optimizing the nucleotide sequence of the V_{L} chain. Except as indicated, introduction of MlyI and BstEII sites is avoided in the process of optimizing the nucleotide sequence of the V_{H} chain.

### Amino Acid Sequences of V_{H} and V_{L} Chains of MOR04945 Optimized for Expression

Codon usage is adapted to that of mammals to enable higher and more stable expression rates in a mammalian cell for the resulting optimized amino acid sequences for the V_{H} and V_{L} chains of the clone MOR04945 described above. See Example 5.

Table 9 below shows the sense (designated “Sense”, SEQ ID NO: 119) and anti-sense (designated “AS”, SEQ ID NO:120) nucleotide sequences of the variable light chain and the resulting variable light chain amino acid (designated “AA”, SEQ ID NO: 121) sequence as optimized for expression.

TABLE 9

| | Nucleotide sense and antisense sequences, and amino acid | |
|---|---|---|
| | sequences of VL chain optimized for expression | |
| | MluI EcoRV BstNI | |
| 1 | ACGCGTTGCGATATCGCCCTGACCCAGCCCGCCAGCGTGTCCGGCAGCCCTGGCCAGAGC | (Sense) |
| | −−−−−−−−−+−−−−−−−−−+−−−−−−−−−+−−−−−−−−−+−−−−−−−−−+−−−−−−−−−+ | |
| | TGCGCAACGCTATAGCGGGACTGGGTCGGGCGGTCGCACAGGCCGTCGGGACCGGTCTCG | (AS) |
| | T__R__C__D__I__A__L__T__Q__P__A__S__V__S__G__S__P__G__Q__S__ | (AA) |
| | PvuII BstNI BstNI | |
| 61 | ATCACCATCAGCTGTACCGGCACCAGCAGCGACCTGGGCGGCTACAACTACGTGTCCTGG | (Sense) |
| | −−−−−−−−−+−−−−−−−−−+−−−−−−−−−+−−−−−−−−−+−−−−−−−−−+−−−−−−−−−+ | |
| | TAGTGGTAGTCGACATGGCCGTGGTCGTCGCTGGACCCGCCGATGTTGATGCACAGGACC | (AS) |
| | I__T__I__S__C__T__G__T__S__S__D__L__G__G__Y__N__Y__V__S__W__ | (AA) |
| 121 | TATCAGCAGCACCCCGGCAAGGCCCCCAAGCTGATGATCTACGACGTGAACAACAGACCT | (Sense) |
| | −−−−−−−−−+−−−−−−−−−+−−−−−−−−−+−−−−−−−−−+−−−−−−−−−+−−−−−−−−−+ | |
| | ATAGTCGTCGTGGGGCCGTTCCGGGGGTTCGACTACTAGATGCTGCACTTGTTGTCTGGA | (AS) |
| | Y__Q__Q__H__P__G__K__A__P__K__L__M__I__Y__D__V__N__N__R__P__ | (AA) |
| | HinfI | |
| 181 | AGCGGCGTGTCCAACAGATTCAGCGGCAGCAAGAGCGGCAACACCGCCAGCCTGACCATC | (Sense) |
| | −−−−−−−−−+−−−−−−−−−+−−−−−−−−−+−−−−−−−−−+−−−−−−−−−+−−−−−−−−−+ | |
| | TCGCCGCACAGGTTGTCTAAGTCGCCGTCGTTCTCGCCGTTGTGGCGGTCGGACTGGTAG | (AS) |
| | S__G__V__S__N__R__F__S__G__S__K__S__G__N__T__A__S__L__T__I__ | (AA) |
| | PstI | |
| 241 | TCTGGCCTGCAGGCTGAGGACGAGGCCGACTACTACTGCCAGACCTACGACCAGATCAAG | (Sense) |
| | −−−−−−−−−+−−−−−−−−−+−−−−−−−−−+−−−−−−−−−+−−−−−−−−−+−−−−−−−−−+ | |
| | AGACCGGACGTCCGACTCCTGCTCCGGCTGATGATGACGGTCTGGATGCTGGTCTAGTTC | (AS) |
| | S__G__L__Q__A__E__D__E__A__D__Y__Y__C__Q__T__Y__D__Q__I__K__ | (AA) |
| | HindIII | |
| 301 | CTGTCCGCCGTGTTTGGCGGCGGAACAAAGCTT (SEQ ID NO: 119) | (Sense) |
| | −−−−−−−−−+−−−−−−−−−+−−−−−−−−−+−−− | |
| | GACAGGCGGCACAAACCGCCGCCTTGTTTCGAA (SEQ ID NO: 120) | (AS) |
| | L__S__A__V__F__G__G__G__T__K__L__ (SEQ ID NO: 121) | (AA) |

Table 10 below shows sense and anti-sense variable heavy chain nucleotide sequences (SEQ ID NO: 122 and 123, respectively) and the resulting variable heavy chain amino acid (designated AA) sequence (SEQ ID NO: 124) as optimized for expression.

TABLE 10

| | Nucleotide sense (designated “Sense”, SEQ ID NO: 122)and | |
|---|---|---|
| | antisense (designated “AS”, SEQ ID NO: 123) sequences, | |
| | and amino acid sequences (designated “AA”, | |
| | SEQ ID NO: 124) of V_{H} chain optimized for expression | |
| | MlyI | |
| | HinfI BstNI PvuII | |
| 1 | GAGTCCATTGGGAGTGCAGGCCCAGGTGCAGCTGGTGGAGAGCGGCGGAGGACTGGTGCA | (Sense) |
| | −−−−−−−−−+−−−−−−−−−+−−−−−−−−−+−−−−−−−−−+−−−−−−−−−+−−−−−−−−−+ | |
| | CTCAGGTAACCCTCACGTCCGGGTCCACGTCGACCACCTCTCGCCGCCTCCTGACCACGT | (AS) |
| | G__V__Q__A__Q__V__Q__L__V__E__S__G__G__G__L__V__Q__ | (AA) |
| | BstNI | |
| 61 | GCCTGGCGGCAGCCTGAGACTGAGCTGTGCCGCCAGCGGCTTCACCTTCAGCAGCTACTG | (Sense) |
| | −−−−−−−−−+−−−−−−−−−+−−−−−−−−−+−−−−−−−−−+−−−−−−−−−+−−−−−−−−−+ | |
| | CGGACCGCCGTCGGACTCTGACTCGACACGGCGGTCGCCGAAGTGGAAGTCGTCGATGAC | (AS) |
| | _P__G__G__S__L__R__L__S__C__A__A__S__G__F__T__F__S__S__Y__W_ | (AA) |
| | BstNI BstNI BclI | |
| 121 | GATGAGCTGGGTGAGGCAGGCCCCTGGCAAGGGCCTGGAGTGGGTGTCCGTGATCAGCAG | (Sense) |
| | −−−−−−−−−+−−−−−−−−−+−−−−−−−−−+−−−−−−−−−+−−−−−−−−−+−−−−−−−−−+ | |
| | CTACTCGACCCACTCCGTCCGGGGACCGTTCCCGGACCTCACCCACAGGCACTAGTCGTC | (AS) |
| | _M__S__W__V__R__Q__A__P__G__K__G__L__E__W__V__S__V__I__S__S_ | (AA) |
| 181 | CGATAGCAGCAGCACCTACTACGCCGATAGCGTGAAGGGCCGGTTCACCATCAGCCGGGA | (Sense) |
| | −−−−−−−−−+−−−−−−−−−+−−−−−−−−−+−−−−−−−−−+−−−−−−−−−+−−−−−−−−−+ | |
| | GCTATCGTCGTCGTGGATGATGCGGCTATCGCACTTCCCGGCCAAGTGGTAGTCGGCCCT | (AS) |
| | _D__S__S__S__T__Y__Y__A__D__S__V__K__G__R__F__T__I__S__R__D_ | (AA) |
| | PstI | |
| | BspMI | |
| 241 | CAACAGCAAGAACACCCTGTACCTGCAGATGAACAGCCTGAGAGCCGAGGACACCGCCGT | (Sense) |
| | −−−−−−−−−+−−−−−−−−−+−−−−−−−−−+−−−−−−−−−+−−−−−−−−−+−−−−−−−−−+ | |
| | GTTGTCGTTCTTGTGGGACATGGACGTCTACTTGTCGGACTCTCGGCTCCTGTGGCGGCA | (AS) |
| | _N__S__K__N__T__L__Y__L__Q__M__N__S__L__R__A__E__D__T__A__V_ | (AA) |
| | BstNI | |
| | BstNI BstNI BstEII | |
| 301 | GTACTACTGTGCCAGGCACGGCATCGACTTCGACCACTGGGGCCAGGGCACCCTGGTCAC | (Sense) |
| | −−−−−−−−−+−−−−−−−−−+−−−−−−−−−+−−−−−−−−−+−−−−−−−−−+−−−−−−−−−+ | |
| | CATGATGACACGGTCCGTGCCGTAGCTGAAGCTGGTGACCCCGGTCCCGTGGGACCAGTG | (AS) |
| | _Y__Y__C__A__R__H__G__I__D__F__D__H__W__G__Q__G__T__L__V__T_ | |
| 361 | C (Sense) (SEQ ID NO: 122) | |
| | — | |
| | G (AS) (SEQ ID NO: 123) | |
| | _ (AA) (SEQ ID NO: 124) | |

Pre- and post-optimization charts may provide the percentages of sequence codons for each of the parental sequences and optimized genes respectively, and analyses the quality class of the respecting nucleotide sequences encoding the V_{H} and V_{L} chains. Quality value as used herein means that the most frequent codon used for a given amino acid in the desired expression system is set as 100, and the remaining codons are scaled accordingly to frequency of usage. (Sharp, P. M., Li, W. H., Nucleic Acids Res. 15 (3), 1987).

Further, the codon adaptation index (CAI) is a number that describes how well the codons of the nucleotide sequence match the codon usage preference of the target organism. The maximum value of CAI is set to 1.0, thus a CAI of >0.9 is considered as enabling high expression. The CAI for the V_{L} chain prior to optimization is found to be 0.73, and after optimization, the CAI is determined to be 0.95. Similarly, the CAI for the V_{H} chain prior to optimization is found to be 0.74, and after optimization, is determined to be 0.98 in optimized constructs, the GC content in the V_{L} chain is increased from 51% for the parent sequence of MOR04945 to 62% for the optimized sequence derived from MOR04945. The GC content in the V_{H} chain is increased from 54% for the parent sequence of MOR04945 to 64% for the optimized derivative of MOR04945.

### Optimization for Expression of Full Length Light Chains and Heavy Chains of MOR04910, MOR04945, MOR04946, and MOR05145

The optimization process is applied to each of the parent full length nucleotide sequences of the light chains of MOR04910 (SEQ ID NO: 97), MOR04945 (SEQ ID NO: 98), MOR04946 (SEQ ID NO: 99), and MOR05145 (SEQ ID NO: 100) and the parent full length nucleotide sequences of the heavy chains of MOR04910 (SEQ ID NO: 101), MOR04945 (SEQ ID NO: 102), MOR04946 (SEQ ID NO: 103), and MOR05145 (SEQ ID NO: 103).

The optimization process is used to construct each of the following light chain nucleotide sequences associated with the parent clone numbers: for clone MOR04910 the optimized nucleotide sequence is SEQ ID NO: 104; for clone MOR04945 the optimized nucleotide sequence is SEQ ID NO: 105; for clone MOR04946 the optimized nucleotide sequence is SEQ ID NO: 106, and for clone MOR05145 the optimized nucleotide sequence is SEQ ID NO: 107. Further, the optimization process is used to construct each of the following heavy chain nucleotide sequences associated with the parent clone numbers: for clone MOR04910 the optimized nucleotide sequence is SEQ ID NO: 108; for clone MOR04945 the optimized nucleotide sequence is SEQ ID NO: 109; for clone MOR04946 the optimized nucleotide sequence is SEQ ID NO: 110; and for clone MOR05145 the optimized nucleotide sequence is SEQ ID NO: 110.

The optimized light chain nucleotide sequences are associated with the following optimized light chain amino acid sequences: for clone MOR04910 the optimized amino acid sequence is SEQ ID NO: 111; for clone MOR04945 the optimized amino acid sequence is SEQ ID NO: 112; for clone MOR04946 the optimized amino acid sequence is SEQ ID NO: 113; and for clone MOR05145 the optimized amino acid sequence is SEQ ID NO: 114. The optimized heavy chain nucleotide sequences are associated with the following optimized heavy chain amino acid sequences: for clone MOR04910 the optimized amino acid sequence is SEQ ID NO: 115; for clone MOR04945 the optimized amino acid sequence is SEQ ID NO: 116; for clone MOR04946 the optimized amino acid sequence is SEQ ID NO: 117; and for clone MOR05145 the optimized amino acid sequence is SEQ ID NO: 117.

A listing of nucleotide and polypeptide sequences of contemplated full length light and heavy chain sequences are provided in Table 11. Table 11 provides optimized nucleotide sequences and the polypeptides encoded by them. These nucleotide sequences are optimized to remove latent splice sites that are recognized in mammalian expression systems.

TABLE 11
Light Chain (LC) and Heavy Chain (HC) Sequences - optimized

LC (opt) 4910 nucleotide
SEQ ID NO: 99
GATATCGCACTGACCCAGCCAGCTTCAGTGAGCGGCTCACCAGGTCAGAGCATTACCATCTCGTGTACGGGTACTAGCAGC
GATGTTGGTGGTTTTAATTATGTGTCTTGGTACCAGCAGCATCCCGGGAAGGCGCCGAAACTTATGATTCATGATGGTTCT
AATCGTCCCTCAGGCGTGAGCAACCGTTTTAGCGGATCCAAAAGCGGCAACACCGCGAGCCTGACCATTAGCGGCCTGCAA
GCGGAAGACGAAGCGGATTATTATTGCCAGTCTTGGGATGTTTCTCCTATTACTGCTGTGTTTGGCGGCGGCACGAAGCTT
ACCGTCCTAGGTCAGCCCAAGGCTGCCCCCTCGGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCAGATAGCAGCCCCGTCAAGGCG
GGAGTGGAGACAACCACACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAAAAGACAGTGGCCCCTACAGAATGT
TCATAG
LC4910 (BHQ880) polypeptide
SEQ ID NO: 100
DIALTQPASVSGSPGQSITISCTGTSSDVGGFNYVSWYQQHPGKAPKLMIHDGSNRPSGVSNRFSGSKSGNTASLTISGLQ
AEDEADYYCQSWDVSPITAVFGGGTKLTVLGQPKAAPSVTLFPPSSEELQANKATLVCLISDFYPGAVTVAWKADSSPVKA
GVETTTPSKQSNNKYAASSYLSLTPEQWKSHRSYSCQVTHEGSTVEKTVAPTECS
LC (opt) 4945 nucleotide
SEQ ID NO: 101
GATATCGCACTGACCCAGCCAGCTTCAGTGAGCGGCTCACCAGGTCAGAGCATTACCATCTCGTGTACGGGTACTAGCAGC
GATCTTGGTGGTTATAATTATGTGTCTTGGTACCAGCAGCATCCCGGGAAGGCGCCGAAACTTATGATTTATGATGTTAAT
AATCGTCCCTCAGGCGTGAGCAACCGTTTTAGCGGATCCAAAAGCGGCAACACCGCGAGCCTGACCATTAGCGGCCTGCAA
GCGGAAGACGAAGCGGATTATTATTGCCAGACTTATGATCAGATTAAGTTGTCTGCTGTGTTTGGCGGCGGCACGAAGCTT
ACCGTCCTAGGTCAGCCCAAGGCTGCCCCCTCGGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCAGATAGCAGCCCCGTCAAGGCG
GGAGTGGAGACAACCACACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAAAAGACAGTGGCCCCTACAGAATGT
TCATAG
LC4945 (BHQ892) polypeptide
SEQ ID NO: 102
DIALTQPASVSGSPGQSITISCTGTSSDLGGYNYVSWYQQHPGKAPKLMIYDVNNRPSGVSNRFSGSKSGNTASLTISGLQ
AEDEADYYCQTYDQIKLSAVFGGGTKLTVLGQPKAAPSVTLFPPSSEELQANKATLVCLISDFYPGAVTVAWKADSSPVKA
GVETTTPSKQSNNKYAASSYLSLTPEQWKSHRSYSCQVTHEGSTVEKTVAPTECS
LC (opt) 4946 nucleotide
SEQ ID NO: 103
GATATCGTGCTGACCCAGAGCCCGGCGACCCTGAGCCTGTCTCCGGGCGAACGTGCGACCCTGAGCTGCAGAGCGAGCCAG
AATCTTTTTTCTCCTTATCTGGCTTGGTACCAGCAGAAACCAGGTCAAGCACCGCGTCTATTAATTTATGGTGCTTCTAAT
CGTGCAACTGGGGTCCCGGCGCGTTTTAGCGGCTCTGGATCCGGCACGGATTTTACCCTGACCATTAGCAGCCTGGAACCT
GAAGACTTTGCGGTGTATTATTGCCAGCAGTATCTTACTCTTCCTCTTACCTTTGGCCAGGGTACGAAAGTCGAGATCAAA
CGAACTGTGGCTGCACCATCTGTCTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCAACAGGGGAGAGTGTTAG
LC4946 (BHQ898) polypeptide
SEQ ID NO: 104
DIVLTQSPATLSLSPGERATLSCRASQNLFSPYLAWYQQKPGQAPRLLIYGASNRATGVPARFSGSGSGTDFTLTISSLEP
EDFAVYYCQQYLTLPLTFGQGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
LC (opt) 5145 nucleotide
SEQ ID NO: 105
GATATCGTGCTGACCCAGAGCCCGGCGACCCTGAGCCTGTCTCCGGGCGAACGTGCGACCCTGAGCTGCAGAGCGAGCCAG
AATCTTTTTTCTCCTTATCTGGCTTGGTACCAGCAGAAACCAGGTCAAGCACCGCGTCTATTAATTTATGGTGCTTCTAAT
CGTGCAACTGGGGTCCCGGCGCGTTTTAGCGGCTCTGGATCCGGCACGGATTTTACCCTGACCATTAGCAGCCTGGAACCT
GAAGACTTTGCGGTGTATTATTGCCAGCAGTATATGACTCTTCCTCTTACCTTTGGCCAGGGTACGAAAGTCGAGATCAAA
CGAACTGTGGCTGCACCATCTGTCTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCICAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCAACAGGGGAGAGTGTTAG
LC5145 (BHQ901) polypeptide
SEQ ID NO: 106
DIVLICSPATLSLSPGERATLSCRASQNLFSPYLAWYQQKPGQAPRLLIYGASNRATGVPARFSGSGSGTDFILTISSLEP
EDFAVYYCQQYMTLPLIFGQGTKVEIKRIVAAPSVFIFPPSDEQLKSGTASVVCLLNNFYPREAKVQWKVDNALQSGNSCE
SVIENSKDSTYSLSSILTLSKADYEKHKVYACEVTHQGLSSPVIKSFNRGEC
HC (opt) 4910 nucleotide
SEQ ID NO: 107
CAGGCACAGGTGCAATTGGTGGAAAGCGGCGGCGGCCTGGTGCAACCGGGCGGCAGCCTGCGTCTGAGCTGCGCGGCCTCC
GGATTTACCTTTTCTTCTTATTGGATGTCTTGGGTGCGCCAAGCCCCTGGGAAGGGTCTCGAGTGGGTGAGCGGTATCTCT
TATTCTGGTAGCAATACCCATTATGCGGATAGCGTGAAAGGCCGTTTTACCATTTCACGTGATAATTCGAAAAACACCCTG
TATCTGCAAATGAACAGCCTGCGTGCGGAAGATACGGCCGTGTATTATTGCGCGCGTATGGGTATTGATCTTGATTATTGG
GGCCAAGGCACCCTGGTCACCGTCTCCTCAGCCTCCACCAAGGGCCCATCGGTCTTCCCCCTGGCACCCTCCTCCAAGAGC
ACCTCTGGGGGCACAGCGGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGCAGCGTCGTGACCGTG
CCCTCCAGCAGCTTGGGCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGGACCGTCAGTCTTCCTC
TTCCCCCCAAAACCCAAGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTGGCTGAATGGCAAGGAGTACAAGTGCAAGGTC
TCCAACAAAGCCCTCCCAGCCCCCATCGAGAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCTGGTCAAAGGCTTCTATCCCACCGACATC
GCCGTGGAGTGGGAGACCAATGGGCACCCGGAGAACAACTACAAGACCACGCCTCCCGTGCTGGACTCCGACCGCTCCTTC
TTCCTCTATACCAAGCTCACCGTGGACAAGAGCAGGTGGCACCAGGGGAACGTCTTCTCATGCTCCGTGATCCATGAGGCT
CTGCACAACCACTACACGCAGAAGAGCCTCTCCCTGTCCCCGGGTAAATGA
HC4910 (BHQ880) polypeptide
SEQ ID NO: 108
QVQLVESGGGLVQPGGSLRLSCAASGFTFSSYWMSWVRQAPCKGLEWVSGISYSGSNTHYADSVKGRFTISRDNSKNTLYL
QMNSLRAEDTAVYYCARMGIDLDYWGQGTLVIVSSASTKGPSVFPLAPSSKSTSGGTAALGCLVKDYFPEPVTVSWNSGAL
TSGVHTFPAVLQSSGLYSLSSVVIVPSSSLGTQTYICNVNHKPSNTKVDKRVEPKSCDKTHICPPCPAPELLGGPSVFLFP
PKPKDTLMISRTPEVICVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLICLVKGFYPSDIAVEWESNGQPENNYKTTPPVLDSDGSFFL
YSKLTVDKSRWQQGNVFSCSVMHEALHNHYTQKSLSLSPCK
HC (opt) 4945 nucleotide
SEQ ID NO: 109
CAGGCACAGGTCCAATTGGTGGAAACCGGCGGCGGCCTGGTGCAACCGGGCGGCACCCTGCGTCTGAGCTGCGCGGCCTCC
GGATTTACCTTTTCTTCTTATTGGATGTCTTGGGTGCGCCAAGCCCCTGGGAAGGGTCTCGAGTGGGTGAGCGTTATCTCT
TCTGATTCTAGCTCTACCTATTATGCGGATACCGTGAAAGGCCGTTTTACCATTTCACGTGATAATTCGAAAAACACCCTG
TATCTGCAAATGAACAGCCTGCGTGCCGAAGATACCGCCGTGTATTATTGCGCGCGTCATGGTATTGATTTTGATCATTGG
GGCCAAGGCACCCTGGTCACCGTCTCCTCAGCCTCCACCAAGGGCCCATCGGTCTTCCCCCTGGCACCCTCCTCCAAGAGC
ACCTCTGGGGGCACACCGGCCCTGGGCTGCCTGGTCAAGGACTACTTCCCCGAACCGCTGACCGTGTCGTGGAACTCAGGC
GCCCTGACCACCGGCGTGCACACCTTCCCGGCTGTCCTACAGTCCTCAGGACTCTACTCCCTCACCACCGTCGTGACCGTG
CCCTCCACCAGCTTGGGCACCCAGACCTACATCTGCAACGTGAATCACAACCCCACCAACACCAAGGTGGACAAGAGAGTT
GAGCCCAAATCTTGTGACAAAACTCACACATGCCCACCGTGCCCACCACCTGAACTCCTGGGGGGACCGTCAGTCTTCCTC
TTCCCCCCAAAACCCAAGGACACCCTCATGATCTCCCGGACCCCTGAGGTCACATGCGTGGTGGTGGACGTGAGCCACGAA
GACCCTGAGGTCAACTTCAACTGGTACGTGGACCGCGTGGAGGTCCATAATGCCAAGACAAAGCCGCGGGAGGACCAGTAC
AACACCACGTACCGTGTGGTCACCGTCCTCACCGTCCTGCACCAGGACTGGCTGAATGGCAAGGAGTACAAGTGCAAGGTC
TCCAACAAAGCCCTCCCAGCCCCCATCGAGAAAACCATCTCCAAAGCCAAAGGGCACCCCCGAGAACCACAGGTGTACACC
CTGCCCCCATCCCGGGAGGAGATGACCAAGAACCAGGTCAGCCTGACCTGCCTGGTCAAAGGCTTCTATCCCACCGACATC
GCCGTGGAGTGGGAGACCAATGGGCACCCGGAGAACAACTACAAGACCACGCCTCCCGTGCTGGACTCCGACCGCTCCTTC
TTCCTCTATACCAAGCTCACCGTGGACAAGAGCAGGTGGCACCAGGGGAACGTCTTCTCATGCTCCGTGATCCATGAGGCT
CTGCACAACCACTACACGCAGAAGAGCCTCTCCCTGTCCCCGGGTAAATGA
HC4945 (BHQ892) polypeptide
SEQ ID NO: 110
QVQLVESGGGLVQPGGSLRLSCAASGFTFSSYWMSWVRQAPCKGLEWVSVISSDSSSTYYADSVKGRFTISRDNSKNTLYL
QMNSLRAEDTAVYYCARHGIDFDHWGQGTLVIVSSASTKGPSVFPLAPSSKSTSGGTAALGCLVKDYFPEPVTVSWNSGAL
TSGVHTFPAVLQSSGLYSLSSVVIVPSSSLGTQTYICNVNHKPSNTKVDKRVEPKSCDKTHICPPCPAPELLGGPSVFLFP
PKPKDILMISRTPEVICVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLICLVKGFYPSDIAVEWESNGQPENNYKTIPPVLDSDGSFFL
YSKLTVDKSRWQQGNVFSCSVMHEALHNHYTQKSLSLSPCK
HC (opt) 4946=5145 nucleotide
SEQ ID NO: 111
CAGGTCCAGCTGGTGGAGAGCGGCGGAGGACTGGTGCACCCTGGCGCCAGCCTGAGACTGAGCTGTGCCGCCACCGGCTTC
ACCTTCAACAACTACCGCATGACCTGGGTGAGGCAGGCCCCTGGCAAGGGCCTGGAGTGGGTGTCCGCCATCACCGCCAGC
GGCAGCTACACCTACTACGCCGACACCGTGAAGGGCAGGTTCACCATCAGCCGGGACAACACCAAGAACACCCTGTACCTG
CAGATGAACAGCCTGAGAGCCGAGGACACCGCCGTGTACTACTGTGCCCGGACCATCTACATGGACTACTGGGGCCAGGGC
ACCCTGGTCACCGTCTCCTCAGCCTCCACCAAGGGCCCATCGGTCTTCCCCCTGGCACCCTCCTCCAAGACCACCTCTGGG
GGCACACCGGCCCTGGGCTGCCTGGTCAAGGACTACTTCCCCGAACCGCTGACCGTGTCGTGGAACTCAGGCGCCCTGACC
ACCGGCGTGCACACCTTCCCGGCTGTCCTACAGTCCTCAGGACTCTACTCCCTCACCACCGTCGTGACCGTGCCCTCCACC
AGCTTGGGCACCCAGACCTACATCTGCAACGTGAATCACAACCCCACCAACACCAAGGTGGACAAGAGAGTTGAGCCCAAA
TCTTGTGACAAAACTCACACATGCCCACCGTGCCCACCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGGACGTGAGCCACGAAGACCCTGAG
GTCAACTTCAACTGGTACGTGGACCGCGTGGAGGTCCATAATGCCAAGACAAAGCCGCGGGAGGACCAGTACAACACCACG
TACCGTGTGGTCACCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCACCCCCGAGAACCACAGGTGTACACCCTGCCCCCA
TCCCGGGAGGAGATGACCAAGAACCAGGTCAGCCTGACCTGCCTGGTCAAAGGCTTCTATCCCACCGACATCGCCGTGGAG
TGGGAGACCAATGGGCAGCCGGAGAACAACTACAAGACCACGCCTCCCGTGCTGGACTCCGACCGCTCCTTCTTCCTCTAT
ACCAACCTCACCGTGGACAAGACCAGGTGGCACCAGGGGAACGTCTTCTCATGCTCCGTGATCCATGAGGCTCTGCACAAC
CACTACACGCAGAAGAGCCTCTCCCTGTCCCCGGGTAAATGA
HC4946=5145 (BHQ898/901) polypeptide
SEQ ID NO: 112
QVQLVESGGGLVQPGGSLRLSCAASGFTFNNYGMTWVRQAPCKGLEWVSGISGSGSYTYYADSVKGRFTISRDNSKNTLYL
QMNSLRAEDTAVYYCARTIYMDYWGQGTLVTVSSASTKGPSVFPLAPSSKSTSCGTAALGCLVKDYFPEPVTVSWNSGALT
SGVHTFPAVLQSSGLYSLSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPCK

### Example 9

### Bioactivity Assays

The biological activity of a neutralizing anti-DKK1/4 antibody is measured in a reporter gene assay, using the genetically modified cell line HEK293 T/17 STF_{—}70 IRES_Krm J17) called SuperTopflash Krm17. This cell line is derived from the human embryonic kidney cell HEK293 and is stably transfected with i) a reporter construct in which the promoter TCF is fused upstream of the firefly luciferase gene and ii) a construct leading to overexpression of Krm on the surface of this cell. In this cell line, exposure to the Wnt protein stimulates the expression of luciferase in a dose-dependent manner. Addition of graded amounts of anti-DKK1/4 antibody to a fixed, sub-maximal dose of DKK1 in the presence of Wnt causes an increase in the expression of luciferase during an incubation period of sixteen hours. At the end of this period, the amount of luciferase is quantified based on its enzymatic activity in the cell lysate. Luciferase catalyses the conversion of the substrate luciferin to oxyluciferin, a chemiluminescent product. The resultant glow-type chemi-luminescence is then determined with an appropriate luminometer.

The biological potency of a neutralizing anti-DKK1/4 antibody test sample is determined by comparing its ability to increase the luciferase expression to that of a reference standard. The samples and standard are normalized on the basis of protein content. Relative potency is then calculated using a parallel line assay according to the European Pharmaco-poeial. The final result is expressed as relative potency (in percent) of a sample compared to the reference standard.

### Example 10

### In Vitro Activity on Relevant Biological Targets

Lead FAbs are selected that have affinities in the low nanomolar range and potent activity in the cellular assay. The physiological binding partners for DKK1 are LRP5/6 (K_{d}˜340 pM) and Kremen 1 and 2 (K_{d}˜280 pM) [Mao 2001] [Mao 2002]. Given these high affinity interactions, it is desirable to further improve affinity in order to better compete with the physiological DKK1 interactions. To increase affinity and biological activity of the selected FAbs, CDR-L3 and CDR-H2 regions are optimized in parallel by cassette mutagenesis using trinucleotide directed mutagenesis [Virnekas 1994][Knappik 2000][Nagy 2002].

Following affinity maturation, a FAb is selected that has low picomolar affinity, reactivates DKK1 inhibited wnt signaling with an EC50 under 1 nM, and cross reacts with cynomolgus monkey, mouse, and rat DKK1. The variable regions of this FAb are then engineered into two different human IgG1 frameworks.

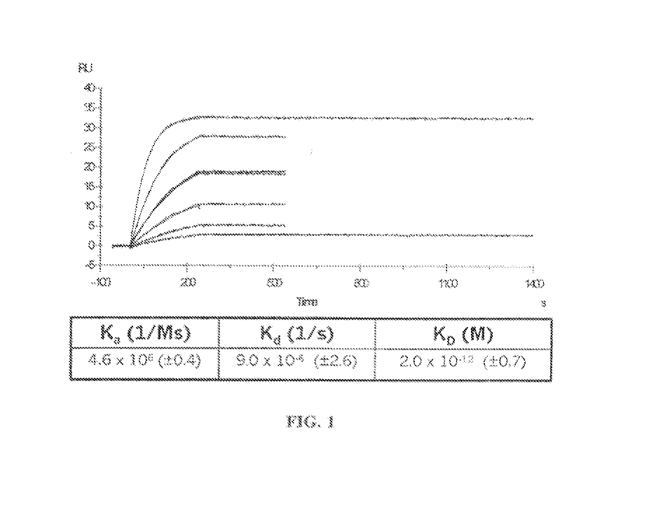

Anti-DKK1/4 antibody has high affinity for human DKK1 (2 pM) with binding kinetics typical for an antibody of this affinity. See FIG. 1.

FIG. 1. METHODS: The binding affinity and kinetics of lead candidates and rhDKK1 (recombinant human DKK1) (Batch BTP7757) are measured using surface plasmon resonance with a Biacore T100 (Biacore, Uppsala, Sweden) instrument containing a CM5 (S) sensor chip (Cat#BR-1006-68). Anti-Human IgG1 Fc (Jackson Immuno Research, Cat#109-006-098) is immobilized onto each flow cell, followed by capture of a lead candidate at an expected capture of about 100 RU. Finally, six concentrations of DKK1 (range 0.195-6.25 nM), with one repeat concentration, is run over the chip. Flow cells are activated for binding of DKK1 for 240 seconds, and disassociation is followed for 30 minutes. The normalized data (background subtracted) are fit to a 1:1 binding with mass transport model using Kinetics analysis in BIA evaluation 1.0 software. This experiment is carried out in triplicate, and data presented are the average of these three experiments with standard deviation.

### Example 11

### Epitope Mapping

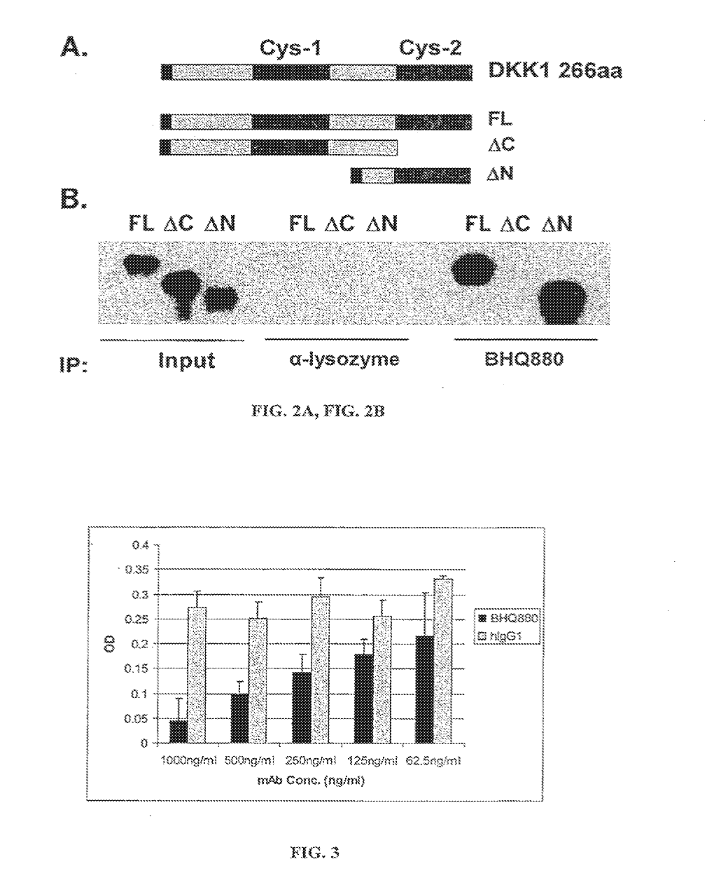

Mature DKK1 is a 266 amino acid protein with two cysteine rich regions (Cys-1 and Cys-2). The Cys-2 domain is responsible for binding both LRPs and Kremen proteins and is necessary and sufficient for inhibition of Wnt signaling [Li 2002][Brott 2002]. Immunoprecipitation experiments (FIG. 2A, 2B) demonstrate that anti-DKK1/4 antibody binds specifically to the Cys-2 domain, but not the Cys-1 domain. anti-DKK1/4 antibody is only weakly active in Western blotting with denatured DKK1 and in a peptide mapping experiment is not found to specifically bind any of the overlapping 15 amino acid peptides covering the length of the protein (JTP), suggesting that anti-DKK1/4 antibody likely recognizes a non-linear epitope within Cys-2.

FIG. 2A shows a schematic representation of full-length and truncated DKK1. Full-length (FL, containing residues 1-266), carboxyl terminal truncated (AC, containing residues 1-185), and amino terminal truncated (AN, containing residues 1-60 plus residues 157-266), are fused with an HA epitope at their C termini, and cloned into a mammalian expression vector under the control of the cytomegalovirus (CMV) promoter. FIG. 2B depicts binding of a neutralizing anti-DKK1/4 antibody and DKK1 proteins. Conditioned medium from transiently transfected Hek293 cells expressing containing full length, amino truncated, carboxyl terminal truncated DKK1 proteins are incubated with anti-lysozyme IgG1 control or the anti-DKK1/4 antibodies for 2 hrs at room temperature, and immunocomplexes are collected on protein G beads, resolved by SDS-PAGE, transferred, and blotted with an anti-HA antibody. 1/10 of total input is loaded as control.

### Example 11A

### Epitope Mapping—N-Glycosylation

A number of proteins within the Wnt signaling pathway are covalently modified by post-translational enzymes which regulate their cellular activity. DKK family members, including DKK1, are modified by N-glycosylation [Krupnik 1999 Gene 238: 301-313]. DKK1 has one theoretical N-linked glycosylation site at amino acid 256 within the Cys-2 domain. Given the highly conserved nature of the Cys-2 domain, and the potential binding site of both DKK1 for LRP6 and anti-DKK1/4 antibody for DKK1 we sought to determine if anti-DKK1/4 antibody recognized the N-glycosylated form of DKK1. An ELISA demonstrates that anti-DKK1/4 antibody recognizes the N-glycosylated form of rhDKK1 much better then the specifically N-linked de-glycosylated form of rhDKK1 TABLE 12A. While the same proteins are recognized equally well with a second antibody (anti-HIS), directed towards the fused epitope tag region of the recombinant protein. This difference in affinity is quantitated by using surface plasmon resonance and found anti-DKK1/4 antibody to have 100 fold higher KD to the glycosylated rhDKK1, then to the de-glycosylated protein, see TABLE 12B.

TABLE 12A
Percent Binding - Glycosylation dependence of anti-DKK1/4
antibody binding to DKK1

| Antibody | WT DKK1 | DKK1 (dyglycosylated) |
|---|---|---|
| anti-HIS tag | 100% | 100% |
| anti-DKK1/4 | 100% | 12-18% |

TABLE 12B
surface plasmon resonance

| Protein | Ka (1/Ms) | Kd (1/s) | KD (M) |
|---|---|---|---|
| WT DKK1 | 7.449E+6 | 2.319E−5 | 3.113E−12 |
| DKK1 (N-degly) | 1.424E+6 | 3.071E−4 | 2.157E−10 |

The binding of anti-DKK1/4 antibody to wild type (WT) rhDKK1 (HEK HIS eiptope tagged Batch# BTP7757) and N-linked deglycosylated (N-DEGLY, N-linked deglycosylated with the enzyme PNGase F (Sigma, Cat# E-DEGLY) rhDKK1 is measured by ELISA. Briefly, a high binding ELISA (Nunc#442404) plate is coated with 1 μg/ml WT or N-DEGLY DKK1. The ratio of both anti-DKK1/4 antibody and anti-HIS antibody binding to WT DKK1 as compared to their respective binding of N-DEGLY is shown. This experiment is carried out with three different concentrations (data for one representative concentration is shown), all concentrations had similar results. B. The binding affinity and kinetics of anti-DKK1/4 antibody to both WT and N-DEGLY DKK1 (HEK293 Batch# BTP7757) are measured using a Biacore T100. anti-DKK1/4 antibody consistently had a 100 fold lower affinity for N-DEGLY DKK1 then it does for WT DKK1.

### Example 12

### Percent Identity of DKK Family Members

The human Dickkopf family consists of four paralogs (see Table 13), three of which (DKK1, 2, & 4) bind to LRP6 and Kremen proteins, induce internalization of LRP5/6 and inhibit canonical Wnt signaling [Mao 2001][Mao 2003]. DKK2 also synergizes with LRP6 overexpression to enhance Wnt signaling, but co-expression of LRP6 and Kremen2 restores DKK2 inhibition of the pathway[Mao 2003]. Thus DKK2 can act as both an agonist and an antagonist depending on the cellular context. DKK3 is the least conserved of the family members, including within the Cys-2 domain responsible for LRP5/6 and Kremen interactions and is distinct from the other DKK family members as it does not bind LRPs or Kremens and does not block Wnt signaling [Mao 2001] [Mao 2003].

TABLE 13
Percent identity of DKK family members across
the whole protein and within the Cys-2 domains.

| | | DKK2 | DKK3 | DKK4 |
|---|---|---|---|---|
| Whole Protein | DKK1 | 38.7 | 15.5 | 32.5 |
| | DKK2 | — | 13.4 | 34.6 |
| | DKK3 | — | — | 15.1 |
| Cys-2 Domain | DKK1 | 69.3 | 23.0 | 56.6 |
| | DKK2 | — | 24.1 | 54.7 |
| | DKK3 | — | — | 20.8 |

Homology among members of the DKK family is evaluated (Vector NTI Advanced 9.1.0) using AlignX algorithm for pairwise sequence alignments comparing ratios of amino acid identities. Gap opening and gap extension penalties of 10 and 0.1 respectively are applied. This evaluation included comparisons of whole proteins, as well as comparisons of Cys-2 domains only. As indicated in the table above, DKKs 1, 2 and 4 share 30-40% amino acid sequence homology across the entire protein. Comparison of Cys-2 domains alone shows DKKs 1 and 2 share 69% homology within this region, while DKK4 shares roughly 57% with the same domain of DKKs 1 and 2. DKK3 shows the lowest level of homology to other family members. Amongst all members homology within the Cys-2 domain is greatest.

### Example 13

### Affinity of Anti-DKK1/4 Antibody for Human DKK Family Members

In addition to binding DKK1, anti-DKK1/4 antibody also binds to DKK4, see Table 14. While the affinity for DKK4 is approximately 100 fold less than for DKK1, it is still subnanomolar and therefore likely biologically and clinically relevant. Of note, neither DKK2 nor DKK4 conserve the Asparagine residue that is predicted to be targeted for glycosylation in the Cys-2 domain of DKK1. Preliminary immunoprecipitation experiments suggest that anti-DKK1/4 antibody does not specifically bind DKK2. The binding affinity of anti-DKK1/4 antibody binding to DKK2 will be determined following successful purification of DKK2. Consistent with the distinct function and binding properties of DKK3, anti-DKK1/4 antibody does not bind DKK3.

TABLE 14
Affinity of anti-DKK1/4 antibody for human DKK family members

| DKK Family Member | K_{D} |
|---|---|
| DKK1 | 2.0 × 10−12M (±0.7) |
| DKK2 | ND |
| DKK3 | NSB |
| DKK4 | 2.97 × 10−10M (±1.5) |

The binding affinity and kinetics of anti-DKK1/4 antibody for other members of human DKK family of proteins are measured using a Biacore T100. As before, experiments are carried out in triplicate for proteins with significant binding to anti-DKK1/4 antibody and are reported as the average of three experiments with standard deviation. DKK3, which is the least homologous family member, did not have binding that is detectable above background levels and so is considered NSB (No Significant binding). Likewise, recent data suggests that an anti-DKK1/4 antibody of the invention also has no significant binding to DKK2.

### Example 14

### Anti-DKK1/4 Antibody Blocks DKK1 Binding to LRP6

DKK1 mediates its Wnt antagonist activity through interactions with LRP5/6 and Kremen, inducing internalization and blocking Wnt induced interaction of LRP5/6 with Frizzled receptors. anti-DKK1/4 antibody competitively inhibits DKK1 binding to LRP6 in a competition ELISA assay in FIG. 3.

HEK293T cells do not express sufficient levels of endogenous LRP5 or 6 to allow visualization of DKK1 binding. However, upon co-transfection of LRP6 with a surface trafficking chaperone protein, MESD, GFP-tagged DKK1 can be detected on the cell surface, illustrating the specific nature of the DKK1/LRP6 interaction. MOR04910, which shares the same variable regions as anti-DKK1/4 antibody, specifically blocks this interaction.

The ability of anti-DKK1/4 antibody to inhibit DKK1 binding directly to LRP6 is measured by ELISA. Briefly, non-treated plates (Fisher, Cat#12565501) are coated with 1 μg/ml of recombinant LRP6 (R&D Systems Cat#1505-LR), then 500 ng/ml of rhDKK1 and a concentration curve of either anti-DKK1/4 antibody or hIgG1 (anti-lysozyme MOR3207, ACE10915) are pre-incubated on ice for 30 minutes after which they are placed onto LRP6 coated plates for 2 hours. Plates are washed and the level of DKK1 binding is detected with anti-DKK1 antibody (R&D Systems AF1096). Shown are the raw OD values (background subtracted). Increasing concentrations of anti-DKK1/4 antibody inhibits DKK1 binding directly to LRP6 in a dose dependent manner, while increasing concentrations of hIgG1 does not block DKK1 binding to LRP6.

The ability of MOR04910 to inhibit DKK1/LRP6 binding on cell surface is measured by fluorescence microscopy. HEK293T cells are either mock transfected and transiently transfected with plasmids encoding LRP6 and MESD. Cells are incubated with DKK1-GFP conditioned medium together with anti-lysozyme FAb or anti-DKK1 FAb MOR04910 for 1 hour at 37° C., and examined by fluorescence microscopy. GFP fluorescence reflects DKK1-GFP binding to overexpressed LRP6 on the plasma membrane. The anti-DKK1/4 antibody blocks DKK1 interactions with LRP6 on cell surfaces.

### Example 14

### Reporter Assays—Reactivation of DKK1 Inhibited TCF/LEF Gene Transcription

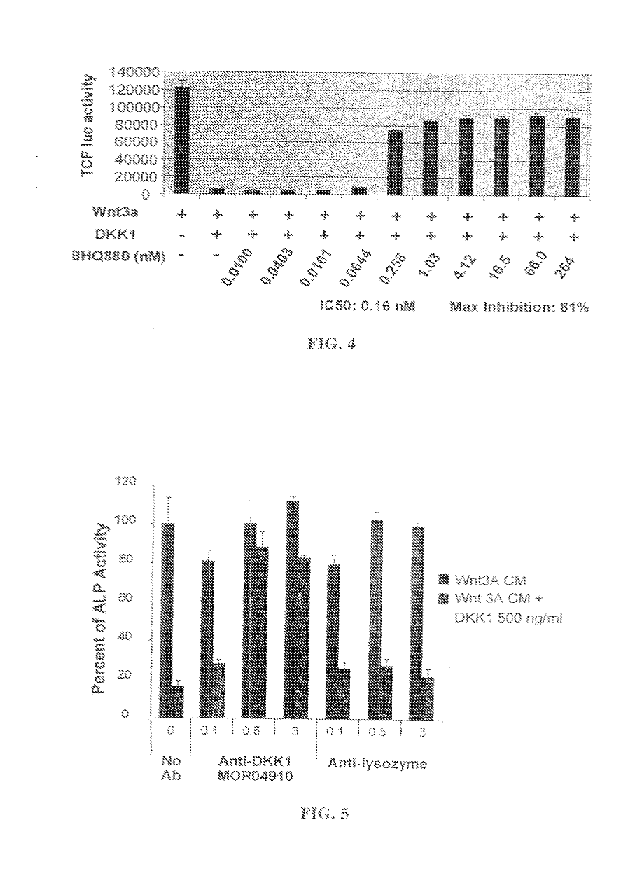

Canonical Wnt signaling culminates in beta-catenin translocation to the nucleus where it associates with transcription factors of the TCF/LEF family resulting in enhanced transcription of Wnt-responsive genes. A reporter assay is established using a TCF/LEF responsive promoter driving Luciferase gene transcription, facilitating detection of Wnt pathway modulation. DKK1 effectively blocks luciferase activity induced by Wnt3A conditioned media (CM) in this assay. Anti-DKK1/4 antibody reactivates DKK1 suppressed Wnt signaling with an apparent EC50 of 0.16 nM FIG. 4. Since the assay requires about 1 nM of DKK1 for complete suppression and the affinity of the antibody is 2 pM, it is likely that this EC50 reflects the sensitivity of the assay and relative amounts of each protein rather than an absolute limit of anti-DKK1/4 antibody competition.

293T cells stably transfected with SuperTopflash reporter and Kremen are treated with 10 ng/ml of rhDKK1, 50% Wnt3a conditioned medium, and various amounts of anti-DKK1/4 antibody antibody. 18 hours later, luciferase activity is measured by the Bright-Glo assay kit (Promega).

### Example 15

### Reporter Assays—Reversal of DKK1 Inhibited Alkaline Phosphatase Secretion in Pre-Osteoblast-Like Cells

To determine whether anti-DKK1/4 antibody blocks DKK1 functions in a more physiological relevant setting, an in vitro assay is established to measure Wnt-mediated osteoblast differentiation of the pluripotent mouse cell line C3H10T1/2 (10T1/2), see FIG. 5. Upon osteoblast differentiation the 10T1/2 cells secrete alkaline phosphatase (AP or ALP), a phenomena which can be inhibited by DKK1. Anti-DKK1/4 antibody, but not IgG control, blocks DKK1 suppression of 10T1/2 differentiation in the presence of Wnt3A conditioned medium.

Wnt has been reported to induce proliferation and inhibit apoptosis in a number of cell contexts and activation of the Wnt pathway, as indicated by beta-catenin stabilization or nuclear localization, is frequently associated with tumor progression. Furthermore, downregulation of DKK1 in some cancers (e.g. colon carcinoma and melanoma) [Gonzalez-Sancho 2005] [Kuphal 2006], has lead some investigators to suggest that DKK1 may be a tumor suppressor for some cancers. To test whether DKK1 has effect on tumor proliferation or survival, tumor cell lines are treated with anti-DKK1/4 antibody and analyzed for changes in growth. No tumor cell line tested is found to be significantly affected by addition of anti-DKK1/4 antibody.

The effect of anti-DKK1/4 antibody on the survival and proliferation of several cancer cell lines is assessed in vitro. In this assay anti-DKK1/4 antibody (100 μg/ml) is incubated with a tumor cell line, after three days cell number is assessed by quantitation of ATP (Promega, Cell Titer Glo Assay®), as a measure of metabolically active cells with a linear relationship to cell number. This assay is carried out in three different serum concentrations (serum free, minimal growth, and complete growth). No significant changes, as compared to untreated and hIgG1 treated cells are found. Cell line supernants are analyzed for DKK1 expression by ELISA.

### Example 16

### Species Crossreactivity and Neutralization of DKK1

A neutralizing anti-DKK1/4 antibody is selected not for its high affinity against human DKK1 and neutralizing ability, but also based upon its crossreactivity with other species that might be used for efficacy and safety studies. anti-DKK1/4 antibody crossreacts with mouse, rat, and cynomolgus monkey (cyno, Macaca fascicularis) DKK1 with similar affinity as for human DKK1, see Table 15. Moreover, anti-DKK1/4 antibody neutralizes all four species' DKK1-mediated Wnt suppressive activity (Table 15), suggesting that these species should be relevant for both safety and efficacy models.

TABLE 15
Species crossreactivity and neutralization of DKK1

| | | Reactivation of wnt3a |
|---|---|---|
| | | signaling (TOPFlash) |
| DKK1 Protein | K_{D} [pM] | EC50 (pM) |
| Human | 17 | 80.6 |
| Cynomolgus | 7 | 54.2 |
| Mouse | 10 | 60.5 |
| Rat | 16 | 255 |

Affinity determination for Human, Cynomolgus, Mouse, and Rat DKK1 is assayed by Solution Equilibrium Titration (SET) using the M-384 SERIES® analyzer (BioVeris, Europe). For KD determination by Solution Equilibrium Titration (SET), monomer fractions (at least 90% monomer content, analyzed by analytical SEC; Superdex75, Amersham Pharmacia) of IgG protein are used. Electrochemiluminescence (ECL) based affinity determination in solution and data evaluation are basically performed as described by [Haenel et al., 2005], the binding fit model is applied as modified according to [Piehler et al., 1997]). A constant amount of MOR4910 IgG is equilibrated with different concentrations (serial 3n dilutions) of human DKK1 (4 nM starting concentration) in solution. Biotinylated human DKK1 coupled to paramagnetic beads (M-280 Streptavidin, Dynal) and BV-tag™ (BioVeris Europe, Witney, Oxfordshire, UK) labelled goat anti-human (Fab)′2 polyclonal antibody is added and incubated for 30 min. Subsequently, the concentration of unbound IgG is quantified via ECL detection using the M-384 SERIES® analyzer (BioVeris, Europe). Affinity determination to rat, mouse, and cynomolgus DKK1 is performed essentially as described above using mouse, rat, and cynomolgus DKK1 as analyte in solution instead of human DKK1. For detection of free IgG molecules, biotinylated human DKK1 coupled to paramagnetic beads is used. MOR4910 and anti-DKK1/4 antibody neutralize human DKK1 (Novartis) with equivalent EC50, anti-DKK1/4 antibody also neutralizes monkey (Novartis), mouse (R&D Systems 1765-DK-010) and rat (Novartis) DKK1. The TOPFLASH reporter assay to human, rat, mouse, and cynomolgus DKK1 is performed essentially as described above (FIG. 4) using each species recombinant DKK1 as the inhibitor of Wnt conditioned media, instead of human DKK1. Rat recombinant DKK1 required higher concentrations of protein to achieve significant inhibition of the TOPFLASH assay.

### Example 17

### Effect of Anti-DKK1/4 Antibody on Intratibial Growth of PC3M2AC6 Xenografts

Prostate tumor metastases are unique among bone metastases in that they are overwhelmingly osteoblastic rather than osteolytic [Keller 2001]. However, even predominantly osteoblastic bone metastases have underlying regions of osteolysis and frequently have low bone mass densities (BMD) especially when patients are on androgen ablation therapy [Saad 2006]. Recently, it is demonstrated that DKK1 can act as a switch, whereby expression of DKK1 enhances osteolytic properties of a mixed osteoblastic/osteolytic prostate tumor cell line (C4-2B). In addition, shRNA suppression of DKK1 inhibited osteolytic activity of a predominantly osteolytic prostate tumor cell line (PC3) [Hall 2005] [Hall 2006]. DKK1 knockdown also inhibited intratibial growth of the tumor xenograft, leading the authors to speculate that osteolytic activity may be important for establishing a metastatic niche, but subsequent loss of DKK1 in prostatic metastases converts the tumor to an osteoblastic phenotype.

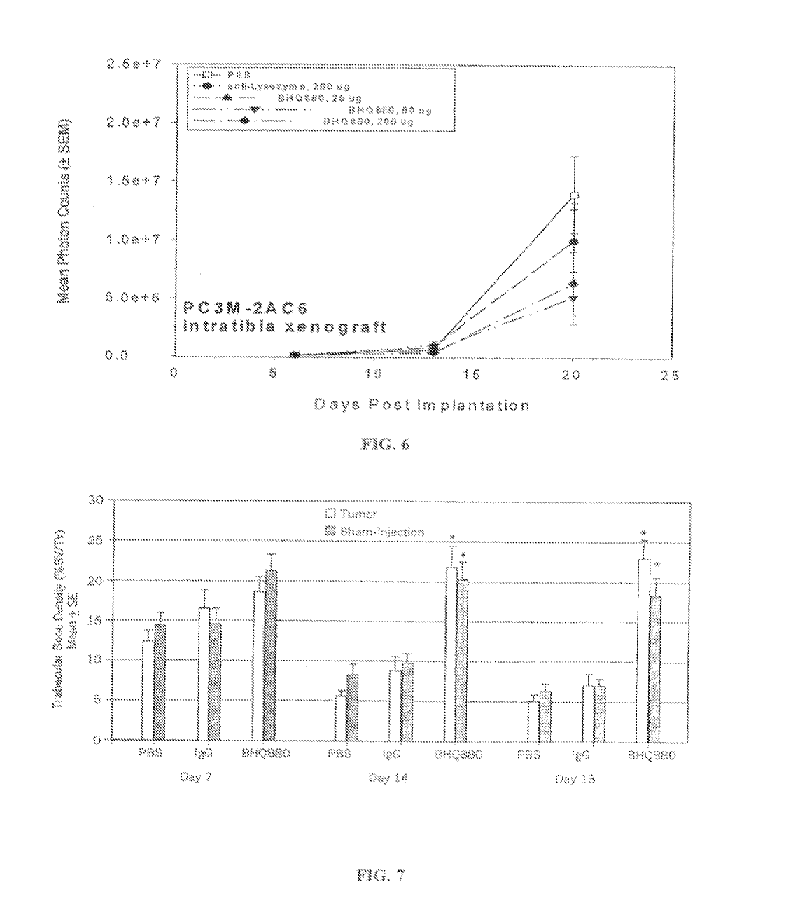

An osteolytic prostate tumor model is adapted from a method by [Kim 2003]. A variant of the osteolytic prostate tumor cell line (PC3M) that stably expresses luciferase (PC3M2AC6) is injected into the tibia of mice. The growth of the tumor is monitored by luciferase while changes in bone are monitored by micro-computerized tomography (micro-CT) and histology. Rather than enhancing tumor growth, anti-DKK1/4 antibody trended toward inhibition of tumor growth. While the inhibition is not significant in any one study, it has occurred consistently in 5/5 studies conducted to date, a representative study showing effects of 3 doses of anti-DKK1/4 antibody on tumor growth is shown FIG. 6. A similar non-significant trend toward inhibition occurred with anti-DKK1/4 antibody treated mice with subcutaneous PC3M2AC6 xenografts.

Treatments are started on day 5 post implantation (0.2 million cells/animal). anti-DKK1/4 antibody is administered i.v., at doses of 20, 60, and 200 μg/mouse/day, q.d., 3 times a week for 2 weeks. Control IgG is administered i.v., at 200 μg/mouse/day, q.d., 3 times a week for 2 weeks. Vehicle control (PBS) is administered i.v., q.d., 3 times a week for 2 weeks. Final efficacy data and body weight change are calculated after treatment.

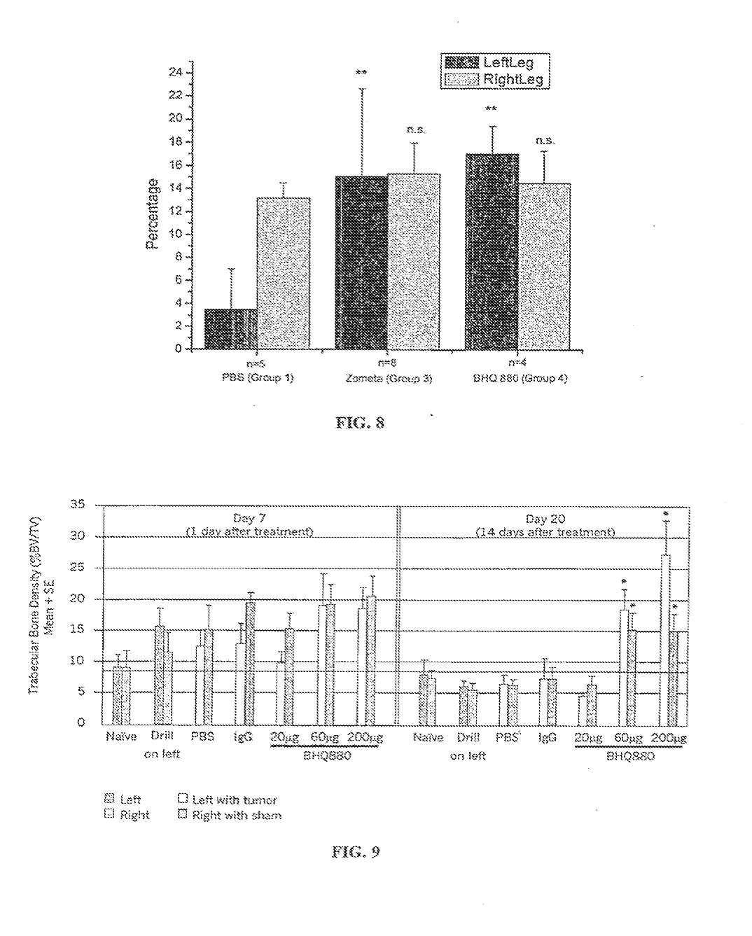

Using this model, we found that an anti-DKK1/4 antibody inhibits tumor-induced cortical bone damage. Effects on trabecular bone are confounded in this model by the observation that both tumor implants and sham implants cause mechanical damage to the bone that result in an initial increase in woven bone which is later remodeled causing a decrease in apparent bone volume. Relative effects of newly formed woven bone and trabeculae on overall bone volume/trabecular volume (BV/TV) ratios are therefore obscured. However, it is clear that anti-DKK1/4 antibody increases the production of bone in both tumor and sham implanted tibias and inhibits or delays the decrease in bone volume accompanying remodeling. Using the same tumor-induced osteolytic model, anti-DKK1/4 antibody demonstrates equivalent anti-osteolytic activity as Zometa, see FIG. 8. The bone metabolic effects of anti-DKK1/4 antibody are dose responsive in the range from 20-200 μg/mouse, with a minimally efficacious dose between 20 and 60 μg/mouse, see FIG. 9. Together these data suggest that anti-DKK1/4 antibody should have an impact in tumor-induced osteolytic disease, but may also be effective in non-tumor bone diseases such as osteoporosis or enhancing repair of bone fractures.

### Example 18

### Anti-DKK1/4 Antibody Maintains Elevated Bone Density in Both Tumor and Sham Implanted Tibias

In an effort to assess pharmocodynamic markers of efficacy in the mice three serum markers of bone metabolism are analyzed: osteocalcin (OC), osteoprotegerin (OPG), and secreted receptor activator of nuclear factor κB ligand (sRANKL). These osteoblast markers are used rather than the more typical osteoclast markers due to the expected mechanism of action of the antibody. However, no consistent changes are detected in animals with tumor versus naïve animals. No correlation of bone loss, as measured by micro-CT or IHC, with any of these markers are consistently observed.

Representative examples of MicroCT reconstructions of the tibias of treated mice are performed. Cortical damage is scored from 0=no damage to 3=severe damage. Cortical damage in tumor-implanted tibias is manually scored by microCT analysis that are blinded with respect to the study groups. No cortical damage is observed in any of the sham implanted legs.

Methods: Female nude mice at age of 12 weeks old are implanted intatibially with 2×10⁵ PC-3M2AC6 cells in the left tibia and sham-injection in the right tibia. Treatments started on day 5 post implantation. NVP-anti-DKK1/4 antibody-NX (anti-DKK1/4 antibody) and IgG control are administered i.v., at doses of 200 μg/mouse/day, q.d., 3 times a week for 2 weeks. Vehicle (PBS) control is also administered i.v., q.d., 3 times a week for 2 weeks. Animals are scanned at day 7, 14, and 18 post tumor implantation using the μ-CT VivaCT40 Scanner (SCANCO, Switzerland). Trabecular bone density (BV/TV) is analyzed as described in methods. An asterisks (*) indicates statistical significant difference from both vehicle and IgG controls (n=12) at the same time point at p<0.05.

In FIG. 7, to determine the bone mass, the secondary spongiosa of the tibia is imaged with the Zeiss Imager Z.1 and Axiovision software based on Giemsa stain. The readout is based on the percent calcified bone in the entire field. Every column represents the mean and standard deviation of the stated number of animals. In the PBS, IgG, and anti-DKK1/4 antibody treated groups, only animals with tumor are analyzed. Right legs did not have sham injections and left legs had tumor. Statistic: Dunnett Multiple Comparisons Test One-Way ANOVA. Left legs or right legs compared to the respective leg in the PBS group p<0.05*, p<0.01**, p>0.05 n.s.

FIG. 9 shows that an anti-DKK1/4 antibody's anabolic bone efficacy is dose dependent with minimal efficacious dose between 20 and 60 μg/mouse 3×/week. Female nude mice at age of 12 weeks old are implanted intratibially with 2×105 PC-3M2AC6 cells in the left tibia and sham-injection in the right tibia. Treatments started on day 6 post implantation. NVP-anti-DKK1/4 antibody-NX (anti-DKK1/4 antibody) is administered i.v., at doses of 20, 60, and 200 μg/mouse/day, q.d., 3 times a week for 2 weeks. Control IgG is administered i.v., at 200 μg/mouse/day, q.d., 3 times a week for 2 weeks. Vehicle control (PBS) is administered i.v., q.d., 3 times a week for 2 weeks. Animals are scanned at day 7 and 20 post tumor implantation using the μ-CT VivaCT40 Scanner (SCANCO, Switzerland). Trabecular bone density (BV/TV) is analyzed, as described in methods. * indicates statistical significant difference from all controls including, vehicle, IgG, drill only, and naïve animals at the same time point at p<0.05.

### Example 19

### Biomarker Status

### DKK1 Biomarkers

The RNA expression pattern of DKK1 has been described. Krupnik (1999) showed expression in placenta by Northern Blot analysis, with no expression detected in heart, brain, lung, liver, skeletal muscle or pancreas. Wirths (2003) showed lack of RNA expression in liver, kidney, and breast, although RNA expression is seen in a subset of hepatoblastomas and Wilms' Tumors. Workers examining gastrointestinal tract expression of DKK1 by RNA in situ hybridization showed no expression in stomach and colon, whether normal or malignant (Byun 2006).

RNA expression analysis in mice revealed high DKK1 expression levels in bone, medium expression in fetus and placenta, and weak expression in brown adipose tissue, thymus and duodenum [Li 2006].

DKK1 protein expression is evaluated in myeloma specimens using the same goat antibody employed in the current study (Tian, 2003). In this paper, expression is seen in myeloma cells of patients with low grade morphology; DKK1 protein expression is not detected in the bone marrow biopsy specimens of five control subjects.

Tissue distribution and species crossreactivity of the therapeutic antibody anti-DKK1/4 antibody is studied by screening it against a series of normal human and monkey tissues. Both whole tissue sections and tissue microarrays are evaluated. Positive controls included a commercial antibody for DKK1 that is evaluated in the same tissue set.

DKK1_{—}15 (FITC conjugated anti-DKK1/4 antibody) and DKK1_{—}8 (FITC conjugated Goat anti-DKK1, R&D Systems, # AF1096, lots GBL013101 and GBL14111).

### Other Biomarkers

Since little is known about the pathophysiologic role of DKK1, a significant amount of effort is and has been focused on building up the knowledge base about the in vivo effects of anti-DKK1/4 antibody by biomarker studies and how this could be exploited to further the development. Key areas of focus have included

1) Understanding the effect of anti-DKK1/4 antibody in normal and metastatic bone metabolism by the measurement of circulating markers of osteoclastic and osteoblastic activity.

2) Comparative expression levels of DKK1 in multiple myeloma and other tumors to confirm and expand target indications.

3) Effects at a gene expression level in key tissues like colon, bone marrow, lung, skin and breast to assess beta-catenin activation.

Preliminary molecular epidemiology studies have confirmed increased DKK1 serum levels in patients with multiple myeloma and support a POC in this indication.

Based on existing knowledge, Table 16 provides the proposed potential Biomarkers for an anti-DKK1/4 antibody.

TABLE 16
Biomarkers for DKK1 and DKK4 targets

| | | | Surrogate |
|---|---|---|---|
| Categories | Tumor | Blood | Tissue |
| Pharmacodynamic | N/A | Free and anti-DKK1/4 Ab bound | Adipose/ |
| (PD) | | DKK-1 levels | skin |
| Target | | Activation of beta-catenin | |
| Downstream | | | |
| Mechanism of Action | | NTx, CTx, PINP, Osteocalcin, | |
| | | RANKL, OPG, PTH, Vitamin D3, | |
| | | calcitonin | |
| Efficacy | Serum M protein, | NTx, CTx, PINP, Osteocalcin, | |
| | Urine total M protein, | RANKL, OPG, PTH, | |
| | b2 microglobin, LDH | calcitoninBone-ALP, CICP, CTx, | |
| | | NTx, etc | |
| Predictive Markers | | DKK-1, CTx, PINP, Osteocalcin, | |
| Stratification | | RANKL, OPG, PTH, Vit D3, | |
| | | calcitonin | |
| Preselection | DKK1 expression | DKK1 serum levels | |
| Safety | | Immunogenicity | |
| Pharmacokinetic | | anti-DKK1/4 Ab | |

### Example 19

### Amino Acid Sequences of Heavy and Light Chain Variable Regions of Anti-DKK1 Antibodies

The amino acid sequences of the variable regions of the light and heavy chains of anti-DKK1 antibodies, whose CDR regions are shown in Tables 5 and 6, are provided in full in Table 17.

TABLE 17
Amino Acid Sequences of Heavy and Light Chain
Variable Regions of anti-DKK1 Antibodies (SEQ ID NOs: 2-39)

MOR04454 VH:
(SEQ ID NO: 2)
QVQLVESGGGLVQPGGSLRLSCAASGFTFSNYGLHWVRQAPGKGLEWVSSISYYGSSTYYADSVKGRFTISRDNSKN
TLYLQMNSLRAEDTAVYYCARDGSHMDKPPGYVFAFWGQGTLVIVSS
MOR04455 VH:
(SEQ ID NO: 3)
QVQLVESGGGLVQPGGSLRLSCAASGFTFSSYGMSWVRQAPGKGLEWVSGISGSGSYTYYADSVKGRFTISRDNSKN
TLYLQMNSLRAEDTAVYYCARHYMDHWGQGTLVIVSS
MOR04456 VH:
(SEQ ID NO: 4)
QVQLVESGGGLVQPGGSLRLSCAASGFTFNNYGMTWVRQAPGKGLEWVSGISGSGSYTYYADSVKGRFTISRDNSKN
TLYLQMNSLRAEDTAVYYCARTIYMDYWGQGTLVIVSS
MOR04461 VH:
(SEQ ID NO: 5)
QVQLVESGGGLVQPGGSLRLSCAASGFTFSSYWMSWVRQAPGKGLEWVSGISYSGSNTHYADSVKGRFTISRDNSKN
TLYLQMNSLRAEDTAVYYCARMGIDLDYWGQGTLVIVSS
MOR04470 VH:
(SEQ ID NO: 6)
QVQLVESGGGLVQPGGSLRLSCAASGFTFSSYWMSWVRQAPGKGLEWVSVISSDSSSTYYADSVKGRFTISRDNSKN
TLYLQMNSLRAEDTAVYYCARHGIDFDHWGQGTLVIVSS
MOR04516 VH:
(SEQ ID NO: 7)
QVQLVQSGAEVKKPGESLKISCKGSGYSFTNYYIGWVRQAPGKGLEWMGIIYPTDSYTNYSPSFQGQVTISADKSIS
TAYLQWSSLKASDTAMYYCARGIPFRMRGFDYWGQGTLVIVSS
MOR04907 VH:
(SEQ ID NO: 8)
QVQLVESGGGLVQPGGSLRLSCAASGFTFNNYGMTWVRQAPGKGLEWVSGISGSGSYTYYADSVKGRFTISRDNSKN
TLYLQMNSLRAEDTAVYYCARTIYMDYWGQGTLVIVSS
MOR04913 VH:
(SEQ ID NO: 9)
QVQLVESGGGLVQPGGSLRLSCAASGFTFNNYGMTWVRQAPGKGLEWVSGISGSGSYTYYADSVKGRFTISRDNSKN
TLYLQMNSLRAEDTAVYYCARTIYMDYWGQGTLVIVSS
MOR04946 VH:
(SEQ ID NO: 10)
QVQLVESGGGLVQPGGSLRLSCAASGFTFNNYGMTWVRQAPGKGLEWVSGISGSGSYTYYADSVKGRFTISRDNSKN
TLYLQMNSLRAEDTAVYYCARTIYMDYWGQGTLVIVSS
MOR04910 VH:
(SEQ ID NO: 11)
QVQLVESGGGLVQPGGSLRLSCAASGFTFSSYWMSWVRQAPGKGLEWVSGISYSGSNTHYADSVKGRFTISRDNSKN
TLYLQMNSLRAEDTAVYYCARMGIDLDYWGQGTLVIVSS
MOR04921 VH:
(SEQ ID NO: 12)
QVQLVESGGGLVQPGGSLRLSCAASGFTFSSYWMSWVRQAPGKGLEWVSGISYSGSNTHYADSVKGRFTISRDNSKN
TLYLQMNSLRAEDTAVYYCARMGIDLDYWGQGTLVIVSS
MOR04948 VH:
(SEQ ID NO: 13)
QVQLVESGGGLVQPGGSLRLSCAASGFIFSSYWMSWVRQAPGKGLEWVSGISYSGSNTHYADSVKGRFTISRDNSKN
TLYLQMNSLRAEDTAVYYCARMGIDLDYWGQGTLVIVSS
MOR04914 VH:
(SEQ ID NO: 14)
QVQLVESGGGLVQPGGSLRLSCAASGFIFSSYWMSWVRQAPGKGLEWVSVISSDSSSTYYADSVKGRFTISRDNSKN
TLYLQMNSLRAEDTAVYYCARHGIDFDHWGQGTLVIVSS
MOR04920 VH:
(SEQ ID NO: 15)
QVQLVESGGGLVQPGGSLRLSCAASGFTFSSYWMSWVRQAPGKGLEWVSSIEHKDAGYTTWYAAGVKGRFTISRDNS
KNTLYLQMNSLRAEDTAVYYCARHGIDFDHWGQGTLVIVSS
MOR04945 VH:
(SEQ ID NO: 16)
QVQLVESGGGLVQPGGSLRLSCAASGFIFSSYWMSWVRQAPGKGLEWVSVISSDSSSTYYADSVKGRFTISRDNSKN
TLYLQMNSLRAEDTAVYYCARHGIDFDHWGQGTLVIVSS
MOR04952 VH:
(SEQ ID NO: 17)
QVQLVESGGGLVQPGGSLRLSCAASGFIFSSYWMSWVRQAPGKGLEWVSVISSDSSSTYYADSVKGRFTISRDNSKN
TLYLQMNSLRAEDTAVYYCARHGIDFDHWGQGTLVIVSS
MOR04954 VH:
(SEQ ID NO: 18)
QVQLVESGGGLVQPGGSLRLSCAASGFIFSSYWMSWVRQAPGKGLEWVSVIEHKDKGGITYYAASVKGRFTISRDNS
KNTLYLQMNSLRAEDTAVYYCARHGIDFDHWGQGTLVIVSS
MOR04947 VH:
(SEQ ID NO: 19)
QVQLVQSGAEVKKPGESLKISCKGSGYSFTNYYIGWVRQAPGKGLEWMGIIVPGTSYTIYSPSFQGQVTISADKSIS
TAYLQWSSLKASDTAMYYCARGIPFRMRGFDYWGQGTLVTVSS
MOR05145 VH:
(SEQ ID NO: 20)
QVQLVESGGGLVQPGGSLRLSCAASGFTFNNYGMTWVRQAPGKGLEWVSGISGSGSYTYYADSVKGRFTISRDNSKN
TLYLQMNSLRAEDTAVYYCARTIYMDYWGQGTLVIVSS
MOR04454 VL:
(SEQ ID NO: 21)
DIQMTQSPSSLSASVGDRVTITCRASQGIKNYLNWYQQKPGKAPKLLIGAASSLQSGVPSRFSGSGSGTDFILTISS
LQPEDFATYYCLQYYGMPPTFGQGTKVEIKRT
MOR04455 VL:
(SEQ ID NO: 22)
DIQMTQSPSSLSASVGDRVTITCRASQDISNYLHWYQQKPGKAPKLLIYGASNLQSGVPSRFSGSGSGTDFILTISS
LQPEDFAVYYCQQYDSIPMTFGQGTKVEIKRT
MOR04456 VL:
(SEQ ID NO: 23)
DIVLIQSPATLSLSPGERATLSCRASQNLFSPYLAWYQQKPGQAPRLLIYGASNRATGVPARFSGSGSGTDFILTIS
SLEPEDFATYYCQQYGDEPITFGQGTKVEIKRT
MOR04461 VL:
(SEQ ID NO: 24)
DIALTQPASVSGSPGQSITISCTWSSDWGFNYVSWYQQHPGKAPKLMIHDGSNRPSWSNRFSGSKSWTASLTI
SGLQAEDEADYYCSTWDMTVDFVFGGGIKLIVLGQ
MOR04470 VL:
(SEQ ID NO: 25)
DI ALT QPASVSGSPGQSITISCTWSSDLGGYNYVSWYQQHPGKAPKLMIYDVNNRPSWSNRFSGSKSWTASLTI
SGLQAEDEADYYCQSYASGNIKVVFGGGIKLIVLGQ
MOR04516 VL:
(SEQ ID NO: 26)
DIVLIQPPSVSGAPGQRVTISCSGSSSNIGSSFVNWYQQLPGTAPKLLIGNNSNRPSGVPDRFSGSKSGTSASLAIT
GLQSEDEADYYCASFDMGSPNVVFGGGIKLIVLGQ
MOR04907 VL:
(SEQ ID NO: 27)
DIVLIQSPATLSLSPGERATLSCRASQNLFSPYLAWYQQKPGQAPRLLIYGASNRATGVPARFSGSGSGTDFILTIS
SLEPEDFAVYYCQQYLSLPTTFGQGTKVEIKRT
MOR04913 VL:
(SEQ ID NO: 28)
DIVLIQSPATLSLSPGERATLSCRASQNLFSPYLAWYQQKPGQAPRLLIYGASNRATGVPARFSGSGSGTDFILTIS
SLEPEDFAVYYCQQYMTLPLIFGQGTKVEINRT
MOR04946 VL:
(SEQ ID NO: 29)
DIVLIQSPATLSLSPGERATLSCRASQNLFSPYLAWYQQKPGQAPRLLIYGASNRATGVPARFSGSGSGTDFILTIS
SLEPEDFAVYYCQQYLTLPLTFGQGTKVEIKRT
MOR04910 VL:
(SEQ ID NO: 30)
DIALTQPASVSGSPGQSITISCIGTSSDVGGFNYVSWYQQHPGKAPKLMIHDGSNRPSGVSNRFSGSKSGNTASLTI
SGLQAEDEADYYCQSWDVSPITAVFGGGIKLIVLGQ
MOR04921 VL:
(SEQ ID NO: 31)
DIALTQPASVSGSPGQSITISCIGTSSDVGGFNYVSWYQQHPGKAPKLMIHDGSNRPSGVSNRFSGSKSGNTASLTI
SGLQAEDEADYYCQTWATSPLSSVFGGGIKLIVLGQ
MOR04948 VL:
(SEQ ID NO: 32)
DIALTQPASVSGSPGQSITISCIGTSSDVGGFNYVSWYQQHPGKAPKLMIHDGSNRPSGVSNRFSGSKSGNTASLTI
SGLQAEDEADYYCQTWDSLSFFVFGGGIKLIVLGQ
MOR04914 VL:
(SEQ ID NO: 33)
DIALTQPASVSGSPGQSITISCIGTSSDLGGYNYVSWYQQHPGKAPKLMIYDVNNRPSGVSNRFSGSKSGNTASLTI
SGLQAEDEADYYCQSYTYTPISPVFGGGIKLIVLGQ
MOR04920 VL:
(SEQ ID NO: 34)
DIALTQPASVSGSPGQSITISCIGTSSDLGGYNYVSWYQQHPGKAPKLMIYDVNNRPSGVSNRFSGSKSGNTASLTI
SGLQAEDEADYYCQSYASGNIKVVFGGGIKLIVLGQ
MOR04945 VL:
(SEQ ID NO: 35)
DIALTQPASVSGSPGQSITISCIGTSSDLGGYNYVSWYQQHPGKAPKLMIYDVNNRPSGVSNRFSGSKSGNTASLTI
SGLQAEDEADYYCQTYDQIKLSAVFGGGTKLTVLGQ
MOR04952 VL:
(SEQ ID NO: 36)
DIALTQPASVSGSPGQSITISCIGTSSDLGGYNYVSWYQQHPGKAPKLMIYDVNNRPSGVSNRFSGSKSGNTASLTI
SGLQAEDEADYYCQSYDSPTDSVVFGGGTKLTVLGQ
MOR04954 VL:
(SEQ ID NO: 37)
DIALTQPASVSGSPGQSITISCIGTSSDLGGYNYVSWYQQHPGKAPKLMIYDVNNRPSGVSNRFSGSKSGNTASLTI
SGLQAEDEADYYCQSYASGNTKVVFGGGTKLIVLGQ
MOR04947 VL:
(SEQ ID NO: 38)
DIVLIQPPSVSGAPGQRVTISCSGSSSNIGSSFVNWYQQLPGTAPKLLIGNNSNRPSGVPDRFSGSKSGTSASLAIT
GLQSEDEADYYCASFDMGSPNVVFGGGTKLTVLGQ
MOR05145 VL:
(SEQ ID NO: 39)
DIVLIQSPATLSLSPGERATLSCRASQNLFSPYLAWYQQKPGQAPRLLIYGASNRATGVPARFSGSGSGTDFTLTIS
SLEPEDFAVYYCQQYMTLPLTFGQGTKVEIKRT

The CDR and FR sections of the variable regions in Table 17 are aligned in Table 18A for heavy chains (SEQ ID NOs:2-20; VH3 is SEQ ID NO:125, VH5 is SEQ ID NO:126), Table 18B for kappa light chains (SEQ ID NOs: 21, 22, 23, 27, 28 and 29; VK1 is SEQ ID NO:127 and VK3 is SEQ ID NO:128), and in Table 18C for lambda light chains (SEQ ID NOs: 24, 25, 30, 31, 32, 33, 34, 35, 36 and 37; VL2 is SEQ ID NO:129 and VL1 is SEQ ID NO:130).

TABLE 18A
Alignment of the Amino Acid Sequences of Heavy Chain Variable Regions of anti-DKK1 Antibodies (SEQ ID NOs: 2-20, 125-126)

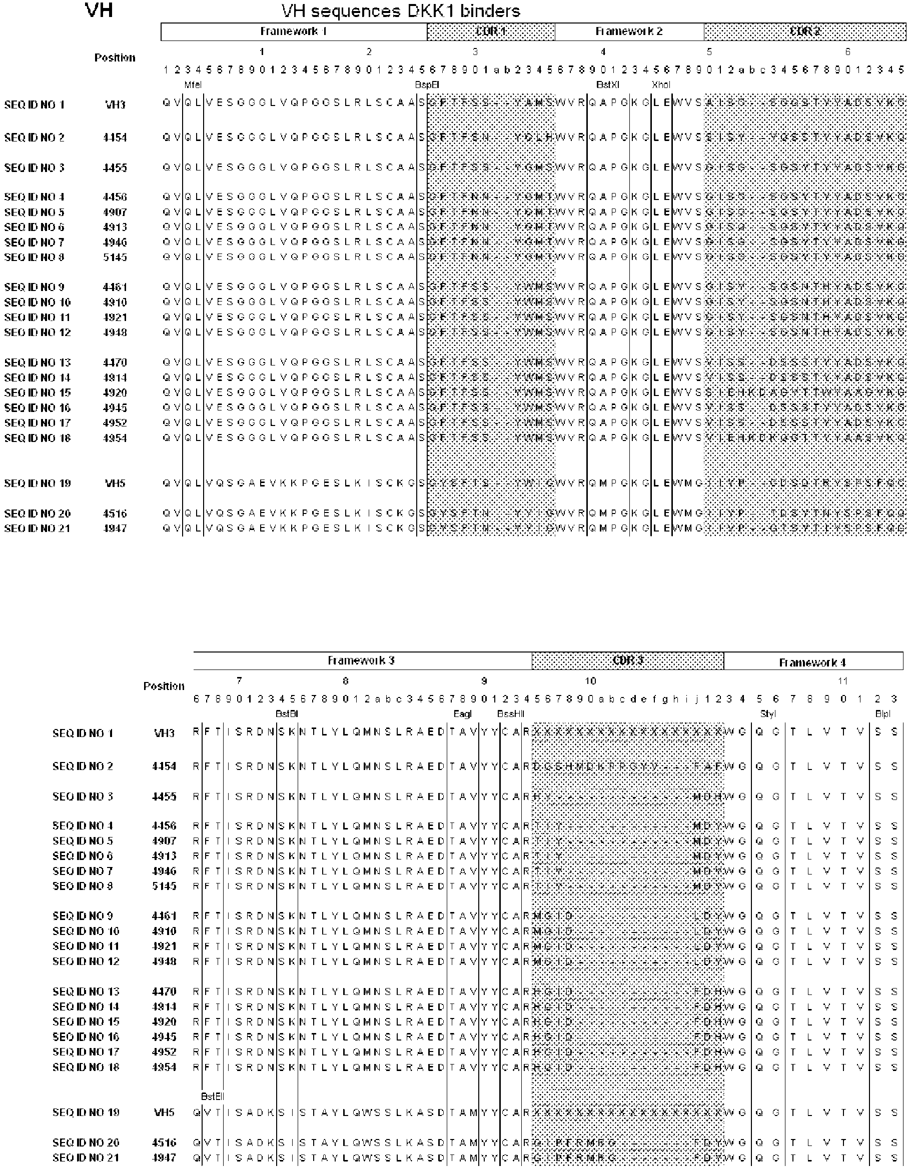

TABLE 18B
Alignment of the Amino Acid Sequences of Kappa Light Chain Variable Regions of anti-DKK1 Antibodies (SEQ ID NOs: 24-25, 30-37, 129-130)

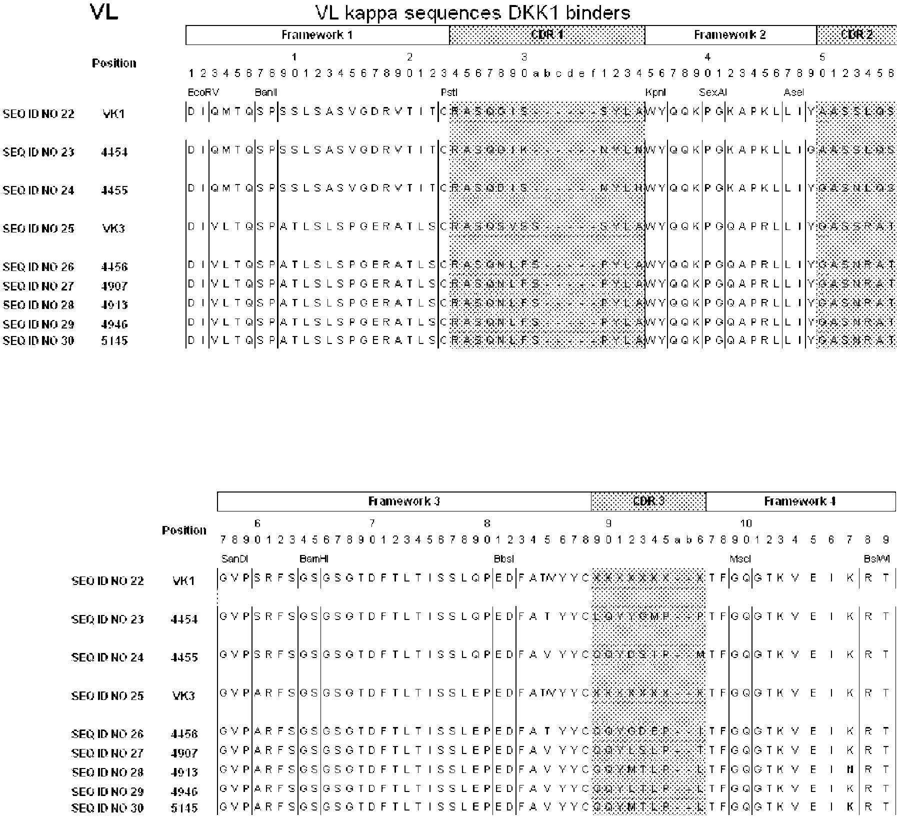

TABLE 18C
Alignment of the Amino Acid Sequences of Lambda Light Chain Variable Regions of anti-DKK1 Antibodies (SEQ ID NOs:)

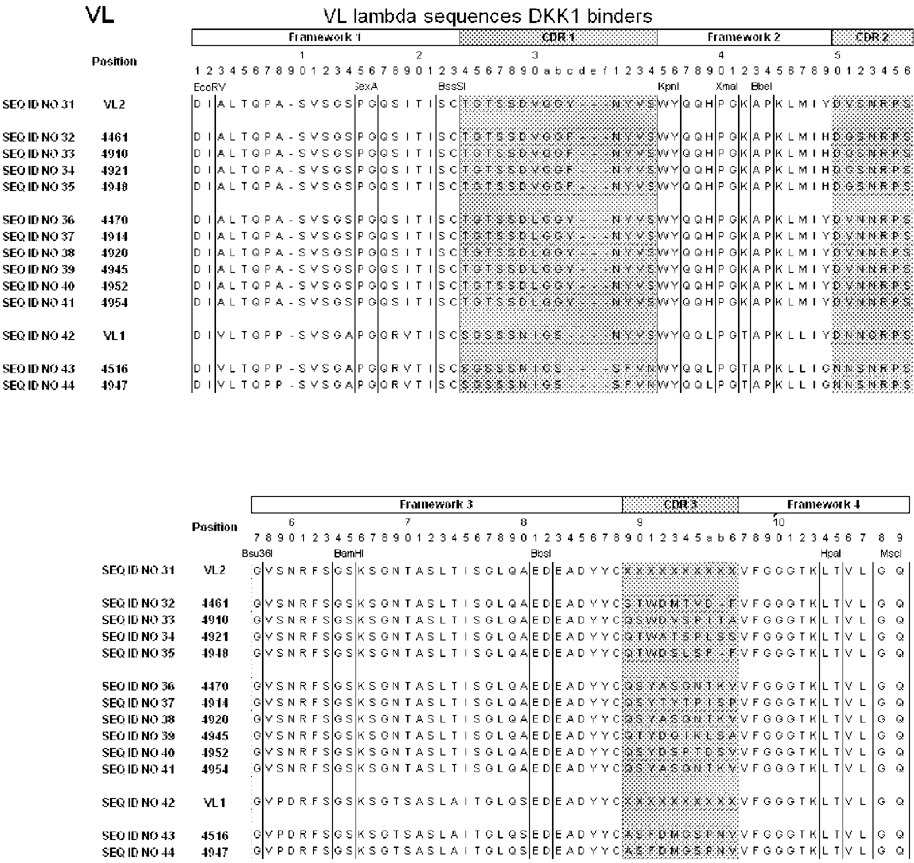

### Example 20

### Anti-DKK1/4 Antibodies in Treating Various Diseases

### A. Evaluation of DKK1/4 Neutralizing Antibody Efficacy

Human mesenchymal stem cells (hMSCs) are progenitors of MFH (malignant fibrous histiocytosis or histiocytoma), as described in Matushanasky et al. 2007 J. Clin. Invest. 117 (11): 3248-3257. DKK1, a mediator of hMSC proliferation, is over-expressed in MFH. DKK1 inhibits hMSC commitment to differentiation via Wnt2/β-catenin canonical signaling. The ability of an anti-DKK1/4 neutralizing antibody to treat MFH and/or inhibit the derivation of sarcomas from hMSCs is evaluated by measuring the effect an antibody of the invention has on the activity levels of those genes and gene products with altered expression in MFH. These markers include nuclear β-catenin, which fails to accumulate in MFH; Wnt2, which is highly over-expressed in MFH; and Wnt5a, which is absent as compared with other sarcoma subtypes. Measurements of alterations of levels and activities of these markers are performed by techniques known in the art.

As described in Matushanasky et al., hMSCs and hMSCs immortalized with SV40 Large T antigen, when grown in medium with DKK1, show tumorigenic colony formation and form tumors when injected into nude mice. In one embodiment, an anti-DKK1/4 antibody is added to the growth medium to evaluate its ability to inhibit derivation of MFH from hMSCs.

In one embodiment, RNA and/or protein levels or activities are evaluated, e.g., by obtaining sample RNA and examining it in a Wnt pathway-specific microarray, as described by You et al. 2008 Dig. Dis. Sci. 53: 1013-1019.

B. Treatment of MFH with a DKK1/4 Antibody

An effective amount of an anti-DKK1/4 antibody of the invention is administered to a subject diagnosed with or at risk for MFH and monitored for therapeutic effect. The presence of metastatic disease is determined with biopsies and CT or MRI scanning. Methods of administration of an antibody are provided here, or may be by methods known to one skilled in the art. These methods include, but are not limited to, i.v. injection in a saline solution or in a solution including about 5% dextrose or glucose in water (“D5W”). Patients are optionally premedicated, e.g., with acetaminophen and diphenhydramine. Dose range (including dose-limited toxicity), safety and pharmacokinetics (including detecting of metabolites and determination of elimination half-life) are determined. Activity of the antibody is determined by measuring levels of DKK1 and nuclear β-catenin, Wnt2, and/or Wnt5a.

Disease treatment also optionally comprises other therapies, including chemotherapy (including ifosfamide and doxorubicin), radiation and surgical excision. Such combination therapy or treatment may be simultaneous, concurrent, separate, or sequential compared to DKK1/4 antibody administration. Patients are monitored throughout treatment for disease state and, after successful treatment, for disease recurrence.

C. Treatment of IBD with a DKK1/4 Antibody

An effective amount of an anti-DKK1/4 antibody of the invention is administered to a subject diagnosed with or at risk for IBD and monitored for therapeutic effect. The presence of IBD (e.g., Crohn's disease and ulcerative colitis) is determined by gastrointestinal inflammation and extra-intestinal manifestations (e.g., liver problems, arthritis, and skin and eye problems). Methods of administration of an antibody are provided here, or may be by methods known to one skilled in the art. These methods include, but are not limited to, i.v. injection in D5W or a saline solution. Patients are optionally premedicated, e.g., with acetaminophen and diphenhydramine. Dose range (including dose-limited toxicity), safety and pharmacokinetics (including detecting of metabolites and determination of elimination half-life) are determined. Activity of the antibody is determined by measuring levels of Wnt genes over-expressed in ulcerative colitis (Wnt2B, Wnt3A, Wnt5B, Wnt6, Wnt7A, Wnt9 and Wnt11).

Disease treatment also optionally comprises other therapies, including surgery and pharmaceutical agents, including immunosuppressive and anti-inflammatory agents, including prednisone, infliximab (Remicade), azathioprine (Imuran), methotrexate, 6-mercaptopurine, and mesalamine. Such combination therapy or treatment may be simultaneous, concurrent, separate, or sequential compared to DKK1/4 antibody administration. Patients are monitored throughout treatment for disease state, including monitoring of symptoms (abdominal pain, vomiting, diarrhea, hematochezia, weight loss, arthritis, pyoderma gangrenosum, and primary sclerosing cholangitis).

D. Treatment of Lung Cancer with a DKK1/4 Antibody

An effective amount of an anti-DKK1/4 antibody of the invention is administered to a subject diagnosed with or at risk for lung cancer (e.g., non-small cell lung cancer) and monitored for therapeutic effect. The presence of lung cancer is determined by chest radiography, bronchoscopy, CT (computed tomography) scan, and/or sputum cytology examination. Methods of administration of an antibody are provided here, or may be by methods known to one skilled in the art. These methods include, but are not limited to, i.v. injection in D5W or a saline solution. Patients are optionally premedicated, e.g., with acetaminophen and diphenhydramine. Dose range (including dose-limited toxicity), safety and pharmacokinetics (including detecting of metabolites and determination of elimination half-life) are determined Activity of the antibody is determined by measuring levels of DKK1, which is over-expressed in this disease.

Disease treatment also optionally comprises other therapies, including surgery, radiotherapy and chemotherapy (e.g., a platinum-based therapy such as cisplatin or carboplatin). Such combination therapy or treatment may be simultaneous, concurrent, separate, or sequential compared to DKK1/4 antibody administration. Patients are monitored throughout treatment for disease state, including monitoring of symptoms (shortness of breath, coughing, coughing up blood, chest or abdominal pain, fatigue, loss of appetite, bone pain, hoarseness, fever, and weight loss).

E. Treatment of Esophageal Squamous Cell Carcinoma (ESCC) with a DKK1/4 Antibody

An effective amount of an anti-DKK1/4 antibody of the invention is administered to a subject diagnosed with or at risk for ESCC and monitored for therapeutic effect. The presence of ESCC is determined by barium swallow, barium meal, esophagogastroduodenoscopy, CT scan, positron emission tomography, and/or esophageal endoscopic ultrasound. Methods of administration of an antibody are provided here, or may be by methods known to one skilled in the art. These methods include, but are not limited to, i.v. injection in D5W or a saline solution. Patients are optionally premedicated, e.g., with acetaminophen and diphenhydramine. Dose range (including dose-limited toxicity), safety and pharmacokinetics (including detecting of metabolites and determination of elimination half-life) are determined. Activity of the antibody is determined by measuring levels of DKK1, which is over-expressed in this disease.

Disease treatment also optionally comprises other therapies, including surgery, laser therapy, radiotherapy and chemotherapy (including fluorouracil and epirubicin, and cisplatin-based compounds, such as carboplatin and oxaliplatin). Such combination therapy or treatment may be simultaneous, concurrent, separate, or sequential compared to DKK1/4 antibody administration. Patients are monitored throughout treatment for disease state, including monitoring of symptoms (including dysphagia and odynophagia, weight loss, vomiting, coughing, pneumonia, hematemesis, pain and poor nutrition).

F. Treatment of Bone Marrow (Skeletal) Metastases with a DKK1/4 Antibody

An effective amount of an anti-DKK1/4 antibody of the invention is administered to a subject diagnosed with or at risk for bone marrow (skeletal) metastases and monitored for therapeutic effect. The presence of bone marrow (skeletal) metastases is determined by biopsy, X-ray analysis, positron emission tomography, bone scan, MRI, and/or scintigraphic imaging. Methods of administration of an antibody are provided here, or may be by methods known to one skilled in the art. These methods include, but are not limited to, i.v. injection in D5W or a saline solution. Patients are optionally premedicated, e.g., with acetaminophen and diphenhydramine. Dose range (including dose-limited toxicity), safety and pharmacokinetics (including detecting of metabolites and determination of elimination half-life) are determined. Activity of the antibody is determined by measuring levels of DKK1, which is over-expressed in this disease.

Disease treatment also optionally comprises other therapies, including surgery, radiotherapy and chemotherapy [including osteoprotegerin; RANKL (receptor activator of nuclear factor-κB) blockers; nuclear factor-κB (NF-κB) antagonists; anti-PTHrP (parathyroid hormone related peptide) antibodies; PDGFR antagonists, such as ST1571 and Imatinib mesylate (Gleevec); ET_{A} (endothelin receptor subtype A) inhibitors, including atrasentan; EMD121974 (cilengitide); matrix metalloproteinase inhibitors; samarium; strontium, and biphosphonates]. Such combination therapy or treatment may be simultaneous, concurrent, separate, or sequential compared to DKK1/4 antibody administration. Patients are monitored throughout treatment for disease state, including monitoring of symptoms, particularly pain.

G. Treatment of Osteosarcoma with a DKK1/4 Antibody

An effective amount of an anti-DKK1/4 antibody of the invention is administered to a subject diagnosed with or at risk for osteosarcoma and monitored for therapeutic effect. The presence of osteosarcoma is determined by biopsy, X-ray analysis, positron emission tomography, bone scan, MRI, and/or scintigraphic imaging. Methods of administration of an antibody are provided here, or may be by methods known to one skilled in the art. These methods include, but are not limited to, i.v. injection in D5W or a saline solution. Patients are optionally premedicated, e.g., with acetaminophen and diphenhydramine. Dose range (including dose-limited toxicity), safety and pharmacokinetics (including detecting of metabolites and determination of elimination half-life) are determined. Activity of the antibody is determined by measuring levels of DKK1, which is over-expressed in this disease.

Disease treatment also optionally comprises other therapies, including surgery chemotherapy (including methotrexate with leucovorin rescue, cisplatin, adriamycin, ifosfamide with mesna, BCD, etoposide, muramyl tri-peptite (MTP). Such combination therapy or treatment may be simultaneous, concurrent, separate, or sequential compared to DKK1/4 antibody administration. Patients are monitored throughout treatment for disease state, including monitoring of symptoms (including pain and tissue necrosis).

### Example 21

3T3-L1 fibroblasts are purchased from ATCC (Catalog # CL173). The cells are grown to confluency and are maintained in DMEM with high glucose (Invitrogen #11995065) supplemented with 10% Fetal Bovine serum and 1% penicillin-streptomycin for an additional 5 days. On the day of differentiation, the culture media is changed to differentiation media supplemented with 11 μg/ml insulin, 115 μg/ml 3-isobuytl-1-methyl xanthine (IBMX) and 0.0975 dexamethasone for 3 days.

The Wnt3a-conditioned media is generated from cells transfected with an expression plasmid encoding Wnt3a. The control conditioned media are generated from cells transfected with an empty vector. On the day of differentiation, Wnt3a-conditioned media containing 11 μg/ml insulin, 115 μg/ml IBMX and 0.0975 μg/ml dexamethasone is added to the cells. On day 3 and 5 of differentiation media is changed to conditioned media. Cells are used for analysis on day 7 of differentiation.

On the day of differentiation various concentrations of Wnt3a alone, Wnt3a with DKK1 or Wnt3a with DKK1 and MOR4910 are added to the differentiation media. Before adding to the cells Wnt3a and DKK1 as well as DKK1 and MOR4910 are combined in a small volume and incubated for 10 minutes on ice. On day 3 and 5 post differentiation, the media is replaced with culture media containing Wnt3a, DKK1 and MOR4910. Cells are used for analysis on day 7 post differentiation. Wnt3a recombinant protein is purchased from R&D systems #GF145.

Preparation of protein lysates and western blotting is performed. Primary antibodies used are goat anti-GLUT4 (Santa Cruz #sc-1608), mouse anti-beta Actin (Abcam #ab6276-100), rabbit anti-phospho AKT (Cell signaling #9271), rabbit anti-AKT (Cell signaling #9272) and anti β-catenin (BD Transduction Lab #610154).

### Example 22

Total RNA is extracted from cells and 1 micro gram of total RNA is used to synthesize cDNA with Superscript III First-Strand synthesis super mix (Invitrogen #18080-400) according to the manufacturer's manual. The newly synthesized cDNA is diluted 1:5 in nuclease free water to a final volume of 100 micro liters and stored at −20 C until used.

Quantitative RT-PCR is performed on an ABI Prism 7900HT Sequence Detection System and analyzed using SDS 2.0 software (Applied Biosystems). One micro liter of cDNA is used in each reaction. The expression of each target gene is normalized by the endogenous control 18S rRNA (Applied Biosystems #4310893E). Assay-on-demand 20× mixes containing specific primers and probe for target genes are obtained from Applied Biosystems.

TABLE 19
Primers and probes used for quantitative real-time PCR

| Target gene | Accession number | Assay-on-demand ID |
|---|---|---|
| 18s RNA | | 4310893E |
| Mouse Axin2 | NM_015732.3 | Mm00443610_m1 |
| Mouse PPARγ | NM_011146.1 | Mm00440945_m1 |
| Mouse C/EBP2 | BC011118.1 | Mm00514283_s1 |
| Mouse FABP4 | NM_024406.1 | Mm00445880_m1 |

### Example 23

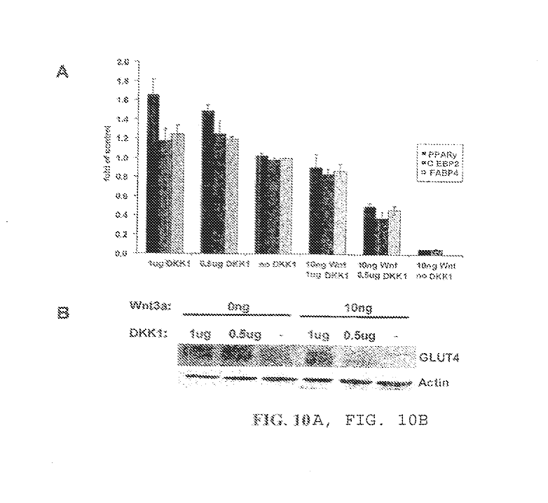

To confirm the effect of DKK1 and Wnt3a on differentiation at the RNA and protein level the following are analyzed: PPARγ (peroxisome proliferators-activated receptor gama), C/EBP2 (CCAAT/enhancer binding protein2) and FABP4 (fatty acid binding protein2) mRNA and GLUT4 (glucose transporter) protein expression. As shown in FIG. 10A treatment with Wnt3a and DKK1 reverses the effect of Wnt3a, therefore mRNA expression level of differentiation markers is increased in these samples. FIG. 10B shows that Wnt3a and DKK1 increase GLUT4 protein expression compared to treatment with Wnt3a alone.

### Example 24

Following the confirmation in accordance with Example 23 that DKK1 can reverse the inhibitory effect of Wnt3a on adipocyte differentiation, it is then tested whether the DKK1 inhibitory antibody MOR4910 can restore the effects of Wnt3a. Cells are treated with differentiation media containing recombinant Wnt3a at 10 ng/ml, DKK1 protein at 1 μg/ml and MOR4910 at 1 micro grams/ml and 2.5 micro grams/ml to the cells. Morphological changes in these cells are monitored as they differentiated, and mRNA expression level of differentiation markers and GLUT4 protein expression is analyzed.

Cells are cultured as described in Example 21. Images are taken on day 4, 5, 6 and 7 to show morphological differences during cell differentiation. As observed previously, inhibition of differentiation by Wnt3a is completely reversed by cotreatment with 1 μg/ml DKK1. The ability of DKK1 to inhibit Wnt3a function is disrupted by addition of 2.5 μg/ml MOR4910. As a result, similar to the cells treated with Wnt3a alone, the cells treated with MOR4910 and DKK1 do not differentiate. Controls show no effect of the control antibody in combination with DKK1 or in combination with Wnt3a and DKK1.

### Example 25

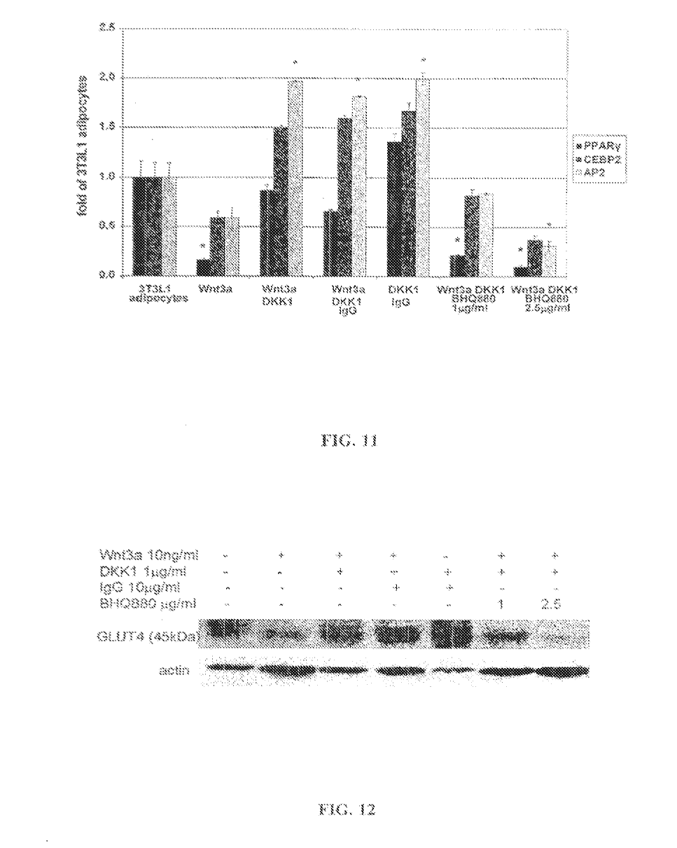

The effect of MOR4910 (labeled “BHQ880” in FIGS. 11 and 12) on differentiation at the RNA and protein level is confirmed by PPARγ, C/EBP2 and FABP4 mRNA and GLUT4 protein expression. As shown in FIG. 11, MOR4910 together with Wnt3a and DKK1 protein reduce expression level of differentiation markers. FIG. 12 shows MOR4910 together with Wnt3a and DKK1 decrease GLUT4 protein expression.

As shown in FIG. 11, total RNA is harvested from the cells treated with Wnt3a, DKK1 and MOR4910 (“BHQ880”). The expression levels of differentiation markers PPARγ, C/EBP2 and AP2 are determined by Q-PCR.

As shown in FIG. 12, lysates from cells are prepared and GLUT4 levels are analyzed with Western blotting. Column 1—expression of Glut 4 in the absence of any additions. Column 2—Wnt3a blocks the expression of Glut4 in 3T3-L1 fibroblasts. Column 3—the addition of DKK-1 in addition to Wnt3a blocks the effects of Wnt3a and causes Glut4 to be expressed. Column 4—the addition of IgG to a combination of DKK-1 and Wnt3a does not effect the expression of Glut4. Column 5—the addition of DKK-1 and IgG to the cells induces Glut4 levels over that of control. Column 6 and 7—addition of 1 ug/ml and 2.5 ug/ml BHQ880 causes the dose-dependent block of Glut4 expression in response to Wnt3a+DKK-1 (compare lanes 3, 6 and 7)—the band for Glut4 decreases in intensity progressively. For all columns, actin shows small variabilities in expression, implying that protein loading is relatively consistent across the columns (lanes).

TABLE 20
Summary of disclosed sequences

| SEQ | | |
|---|---|---|
| ID NO. | Type | Description |
| 1 | PRT | Human DKK1 full length protein |
| 2 | PRT | Heavy Chain Variable Region-VH3 MOR04454 |
| 3 | PRT | Heavy Chain Variable Region-VH3 MOR04455 |
| 4 | PRT | Heavy Chain Variable Region-VH3 MOR04456 |
| 5 | PRT | Heavy Chain Variable Region-VH3 MOR04461 |
| 6 | PRT | Heavy Chain Variable Region-VH3 MOR04470 |
| 7 | PRT | Heavy Chain Variable Region-VH5 MOR04516 |
| 8 | PRT | Heavy Chain Variable Region-VH3 MOR04907 |
| 9 | PRT | Heavy Chain Variable Region-VH3 MOR04913 |
| 10 | PRT | Heavy Chain Variable Region-VH3 MOR04946 |
| 11 | PRT | Heavy Chain Variable Region-VH3 MOR04910 |
| 12 | PRT | Heavy Chain Variable Region-VH3 MOR04921 |
| 13 | PRT | Heavy Chain Variable Region-VH3 MOR04948 |
| 14 | PRT | Heavy Chain Variable Region-VH3 MOR04914 |
| 15 | PRT | Heavy Chain Variable Region-VH3 MOR04920 |
| 16 | PRT | Heavy Chain Variable Region-VH3 MOR04945 |
| 17 | PRT | Heavy Chain Variable Region-VH3 MOR04952 |
| 18 | PRT | Heavy Chain Variable Region-VH3 MOR04954 |
| 19 | PRT | Heavy Chain Variable Region-VH5 MOR04947 |
| 20 | PRT | Heavy Chain Variable Region-VH3 MOR05145 |
| 21 | PRT | Kappa Light Chain Variable Region-VK1 MOR04454 |
| 22 | PRT | Kappa Light Chain Variable Region-VK1 MOR04455 |
| 23 | PRT | Kappa Light Chain Variable Region-VK3 MOR04456 |
| 24 | PRT | Lambda Light Chain Variable Region-VL2 MOR04470 |
| 25 | PRT | Lambda Light Chain Variable Region-VL2 MOR04461 |
| 26 | PRT | Lambda Light Chain Variable Region-VL1 MOR04516 |
| 27 | PRT | Kappa Light Chain Variable Region-VK3 MOR04907 |
| 28 | PRT | Kappa Light Chain Variable Region-VK3 MOR04913 |
| 29 | PRT | Kappa Light Chain Variable Region-VK3 MOR04946 |
| 30 | PRT | Lambda Light Chain Variable Region-VL2 MOR04910 |
| 31 | PRT | Lambda Light Chain Variable Region-VL2 MOR04921 |
| 32 | PRT | Lambda Light Chain Variable Region-VL2 MOR04948 |
| 33 | PRT | Lambda Light Chain Variable Region-VL2 MOR04914 |
| 34 | PRT | Lambda Light Chain Variable Region-VL2 MOR04920 |
| 35 | PRT | Lambda Light Chain Variable Region-VL2 MOR04945 |
| 36 | PRT | Lambda Light Chain Variable Region-VL2 MOR04952 |
| 37 | PRT | Lambda Light Chain Variable Region-VL2 MOR04954 |
| 38 | PRT | Lambda Light Chain Variable Region-VL1 MOR04947 |
| 39 | PRT | Kappa Light Chain Variable Region-VK3 MOR05145 |
| 40 | PRT | Consensus H-CDR1 from TABLE 18a-GFTFNNYGMT |
| 41 | PRT | Consensus H-CDR1 from TABLE 18a-GFTFSSYWMT |
| 42 | PRT | Consensus H-CDR1 from TABLE 18a-GFTF(S/N)(S/N)Y(G/W)X(S/T/X) |
| 43 | PRT | Consensus H-CDR1 from TABLE 18a-GYSFTNYYIG |
| 44 | PRT | Consensus H-CDR2 from TABLE 18a-GISGSGSYTYYADSVKG |
| 45 | PRT | Consensus H-CDR2 from TABLE 18a-GISYSGSNTHYADSVKG |
| 46 | PRT | Consensus H-CDR2 from TABLE 18a-VISSDSSSTYYADSVKG |
| 47 | PRT | Consensus H-CDR2 from TABLE 18a-II(Y/V)PXXSYT(N/I)YSPSFQG |
| 48 | PRT | Consensus H-CDR3 from TABLE 18a-X(G/X)I(D/Y)XD(Y/H) |
| 49 | PRT | HCDR1-VH3-GFTFSSYGMS |
| 50 | PRT | HCDR1-VH3-GFTFNNYGMT |
| 51 | PRT | HCDR1-VH3-GFTFSSYWMS |
| 52 | PRT | HCDR1-VH5-GYSFTNYYIG |
| 53 | PRT | HCDR2-VH3-WVSGISGSGSYTYYADSVKG |
| 54 | PRT | HCDR2-VH3-WVSGISERGVYIFYADSVKG |
| 55 | PRT | HCDR2-VH3-WVSGISYSGSNTHYADSVKG |
| 56 | PRT | HCDR2-VH3-WVSDIEHKRRAGGATSYAASVKG |
| 57 | PRT | HCDR2-VH3-WVSMIEHKTRGGTTDYAAPVKG |
| 58 | PRT | HCDR2-VH3-WVSVISSDSSSTYYADSVKG |
| 59 | PRT | HCDR2-VH3-WVSVIEHKSFGSATFYAASVKG |
| 60 | PRT | HCDR2-VH3-WVSVIEHKDKGGTTYYAASVKG |
| 61 | PRT | HCDR2-VH3-WVSSIEHKDAGYTTWYAAGVKG |
| 62 | PRT | HCDR2-VH5-WMGIIYPTDSYTNYSPSFQG |
| 63 | PRT | HCDR2-VH5-WMGIIYPGTSYTIYSPSFGQ |
| 64 | PRT | HCDR3-VH3-HYMDH |
| 65 | PRT | HCDR3-VH3-TIYMDY |
| 66 | PRT | HCDR3-VH3-MGIDLDY |
| 67 | PRT | HCDR3-VH3-HGIDFDH |
| 68 | PRT | HCDR3-VH5-GIPFRMRGFDY |
| 69 | PRT | HCDR3-VH3-DGSHMDKPPGYVFAF |
| 70 | PRT | LCDR1-VK1-RASQDISNYLH |
| 71 | PRT | LCDR1-VK3-RASQNLFSPYLA |
| 72 | PRT | LCDR1-VL2-TGTSSDVGGFNYVS |
| 73 | PRT | LCDR1-VL2-TGTSSDLGGYNYVS |
| 74 | PRT | LCDR1-VL1-SGSSSNIGSSFVN |
| 75 | PRT | LCDR2-VK1-LLIYGASNLQS |
| 76 | PRT | LCDR2-VK3-LLIYGASNRAT |
| 77 | PRT | LCDR2-VL2-LMIHDGSNRPS |
| 78 | PRT | LCDR2-VL2-LMIYDVNNRPS |
| 79 | PRT | LCDR2-VL1-LLIGNNSNRPS |
| 80 | PRT | LCDR3-VK1-LQYYGMPP |
| 81 | PRT | LCDR3-VK1-QQYDSIPM |
| 82 | PRT | LCDR3-VK3-QQYGDEPI |
| 83 | PRT | LCDR3-VK3-QQYLSLPT |
| 84 | PRT | LCDR3-VK3-QQYLTLPL |
| 85 | PRT | LCDR3-VK3-QQYLFPL |
| 86 | PRT | LCDR3-VK3-QQYMTLPL |
| 87 | PRT | LCDR3-VL2-STWDMTVDF |
| 88 | PRT | LCDR3-VL2-QSWDVSPITA |
| 89 | PRT | LCDR3-VL2-QTWDSLSFF |
| 90 | PRT | LCDR3-VL2-QSWGVGPGGF |
| 91 | PRT | LCDR3-VL2-QTWATSPLSS |
| 92 | PRT | LCDR3-VL2-QSYASGNTKV |
| 93 | PRT | LCDR3-VL2-QSYTYTPISP |
| 94 | PRT | LCDR3-VL2-QTYDQIKLSA |
| 95 | PRT | LCDR3-VL2-QSYDPFLDVV |
| 96 | PRT | LCDR3-VL2-QSYDSPTDSV |
| 97 | PRT | LCDR3-VL2-QSYASGNTKV |
| 98 | PRT | LCDR3-VL1-ASFDMGSPNV |
| 99 | DNA | Optimized LC (opt) 4910 nucleotide |
| 100 | PRT | Optimized LC4910 (BHQ880) polypeptide |
| 101 | DNA | Optimized LC (opt) 4945 nucleotide |
| 102 | PRT | Optimized LC4945 (BHQ892) polypeptide |
| 103 | DNA | Optimized LC (opt) 4946 nucleotide |
| 104 | PRT | Optimized LC4946 (BHQ898) polypeptide |
| 105 | DNA | Optimized LC (opt) 5145 nucleotide |
| 106 | PRT | Optimized LC5145 (BHQ901) polypeptide |
| 107 | DNA | Optimized HC (opt) 4910 nucleotide |
| 108 | PRT | Optimized HC4910 (BHQ880) polypeptide |
| 109 | DNA | Optimized HC (opt) 4945 nucleotide |
| 110 | PRT | Optimized HC4945 (BHQ892) polypeptide |
| 111 | DNA | Optimized HC (opt) 4946=5145 nucleotide |
| 112 | PRT | Optimized HC4946=5145 (BHQ898/901) polypeptide |
| 113 | PRT | VL consensusl CDR1-RASQxxxxxYx |
| 114 | PRT | VL consensusl CDR2-LLIYGASNxxx |
| 115 | PRT | VL consensusl CDR3-QQYxxxPx |
| 116 | PRT | VL consensus2 CDR1-TGTSSDVGGFNYVS |
| 117 | PRT | VL consensus2 CDR2-LMIxDxxNRPS |
| 118 | PRT | VL consensus2 CDR3-xxWDxxxxx |
| 119 | DNA | Optimized VL chain nucleotide-sense strand |
| 120 | DNA | Optimized VL chain nucleotide-antisense strand |
| 121 | PRT | Optimized VL chain polypeptide |
| 122 | DNA | Optimized VH chain nucleotide-sense strand |
| 123 | DNA | Optimized VH chain nucleotide-antisense strand |
| 124 | PRT | Optimized VH chain polypeptide |
| 125 | PRT | VH3 scaffold from FIG. 18 |
| 126 | PRT | VH5 scaffold from FIG. 18 |
| 127 | PRT | VK1 scaffold from FIG. 18 |
| 128 | PRT | VK3 scaffold from FIG. 18 |
| 129 | PRT | VL2 scaffold from FIG. 18 |
| 130 | PRT | VL1 scaffold from FIG. 18 |
| 131 | PRT | Human DKK2 full polypeptide |
| 132 | PRT | Human DKK3 full polypeptide |
| 133 | PRT | Human DKK4 full polypeptide |

### EQUIVALENTS

From the foregoing detailed description of the specific embodiments of the invention, it should be apparent that novel antibodies and immunological fragments thereof have been described. Although particular embodiments have been disclosed herein in detail, this has been done by way of example for purposes of illustration only. In particular, it is contemplated by the inventor that various substitutions, alterations, and modifications may be made to the invention without departing from the spirit and scope of the invention.

What is claimed is:

1. A method for treating a disorder or condition associated with the presence of DKK1 and/or DKK4, comprising administering to a subject in need thereof an effective amount of the pharmaceutical composition for use as a medicament, the composition comprising an antigen-binding region that specifically binds an epitope in a DKK1 polypeptide (SEQ ID NO: 1) and/or in a DKK4 polypeptide (SEQ ID NO: 131), wherein the antibody or functional fragment thereof binds to at least one epitope in DKK1 or DKK4 or both, and the medicament is for use in treating a disorder or condition associated with the presence of DKK1 and/or DKK4.

2. A method for treating a disorder or condition associated with the presence of DKK1 and/or DKK4, wherein the disorder or condition is selected from:
i. malignant fibrous histiocytosis (MFH);
ii. beta thalassemia;
iii. neuroblastoma;
iv. inflammatory bowel disease and irritable bowel syndrome;
v. type 2 diabetes mellitus;
vi. glucocorticoid or other drug associated diabetes;
vii. non-insulin dependent diabetes mellitus;
viii. hypoinsulinemia;
ix. disorders related to pigmentation;
x. cardiovascular disorders;
xi. a cholesterol-related disorder;
xii. MGUS;
xiii. plateau myeloma; and
xiv. smoldering myeloma,
the method comprising administering to a subject in need thereof a pharmaceutically effective amount of a DKK1/4 antibody comprising the CDR1, CDR2 and CDR3 regions selected from Tables 5, 6, 7 and 8.

3. The method according to claim 2, wherein the method further comprises administering a second therapeutic agent.

4. The method according to claim 3, wherein the second therapeutic agent is selected from an anti-cancer agent; an anti-osteoporotic agent; an antibiotic; an antimetabolic agent; an antidiabetic agent; an anti-inflammatory agent; an anti-angiogenic agent; a growth factor; a bone anabolic, a weight loss therapy, a hypylipidemic agent, and anti-obesity agent, an anti-hypertensive agent, and/or an agonist of peroxisome proliferators-activator receptors (PPARs) and a cytokine.

5. The method of claim 3, wherein the second therapeutic agent is a pharmaceutically active agent other than a neutralizing anti-DKK1/4 composition or a derivative thereof, which agent is selected from:
i. an aromatase inhibitor;
ii. an anti-estrogen, an anti-androgen or a gonadorelin agonist;
iii. a topoisomerase I inhibitor or a topoisomerase II inhibitor;
iv. a microtubule active agent, an alkylating agent, an anti-neoplastic anti-metabolite or a platin compound;
v. a compound targeting/decreasing a protein or lipid kinase activity or a protein or lipid phosphatase activity, a further anti-angiogenic compound or a compound which induces cell differentiation processes;
vi. monoclonal antibodies;
vii. a cyclooxygenase inhibitor, a bisphosphonate, a heparanase inhibitor, a biological response modifier;
viii. an inhibitor of Ras oncogenic isoforms;
ix. a telomerase inhibitor;
x. a protease inhibitor, a matrix metalloproteinase inhibitor, a methionine aminopeptidase inhibitor, or a proteasome inhibitor;
xi. agents used in the treatment of hematologic malignancies or compounds which target, decrease or inhibit the activity of Flt-3;
xii. an HSP90 inhibitor;
xiii. antiproliferative antibodies;
xiv. a histone deacetylase (HDAC) inhibitor;
xv. a compound which targets, decreases or inhibits the activity/function of serine/threonine mTOR kinase;
xvi. a somatostatin receptor antagonist;
xvii. an anti-leukemic compound;
xviii. tumor cell damaging approaches;
xix. an EDG binder;
xx. a ribonucleotide reductase inhibitor;
xxi. an S-adenosylmethionine decarboxylase inhibitor;
xxii. a monoclonal antibody of VEGF or VEGFR;
xxiii. photodynamic therapy;
xxiv. an angiostatic steroid;
xxv. an implant containing corticosteroids;
xxvi. an AT1 receptor antagonist;
xxvii. an ACE inhibitor;
xxviii. an antidiabetic agent;
xxix. a hypolipidemic agent;
xxx. an anti-obesity agent;
xxxi. an anti-hypertensive agent; and
xxxii. an agonist of peroxisome proliferators-activator receptors (PPARs);
and optionally a pharmaceutically acceptable carrier

6. A method for treating malignant Fibrous histiocytosis (MFH) comprising administering to a subject in need thereof a pharmaceutically effective amount of a DKK1/4 antibody comprising the CDR1, CDR2 and CDR3 regions selected from Tables 5, 6, 7 and 8.

7. A method of claim 2, wherein the bone disorder is selected from the group consisting of: bone fracture healing, osteolytic lesions and metastases, osteopenia, osteoporosis, bone density abnormality, osteosarcoma, and osteolysis.

8. A method of claim 2, wherein the cancer is selected from the group consisting of:
myeloma, multiple myeloma, MGUS, smoldering or plateau myeloma; a cancer of the bone, breast, colon, melanocytes, hepatocytes, hepatocellular carcinoma (HCC), epithelium, esophagus, brain, lung, prostate or pancreas; or metastasis thereof.

9. A method of claim 2, wherein the muscle disease is selected from the group consisting of: muscle trauma, atrophy, wasting, degeneration, repair, regeneration.

10. A method of claim 2, wherein the metabolic disease is selected from the group consisting of: insulin resistance, non-insulin-dependent diabetes mellitus (NIDDM), hypoinsulinemia, diabetes (especially type 2 diabetes mellitus, or glucocorticoid or other drug associated diabetes), obesity, weight loss, weight loss maintenance, anorexia nervosa, bulimia, cachexia, syndrome X, metabolic syndrome, post-prandial hyperglycemia, post prandial hyperlipidemia and/or hypertriglyceridemia, hypoglycemia, hyperglycemia, hyperuricemia, hyperinsulinemia, hypercholesterolemia, hyperlipidemia, dyslipidemia, mixed dyslipidemia, hypertriglyceridemia, pancreatitis, and nonalcoholic fatty liver disease.

11. A method of claim 2, wherein the cardiovascular disease is selected from the group consisting of: coronary artery disease, vascular calcification, claudication, atherosclerosis, arteriosclerosis, acute heart failure, congestive heart failure, coronary artery disease, cardiomyopathy, myocardial infarction, angina pectoris, hypertension, hypotension, stroke, ischemia, ischemic reperfusion injury, aneurysm, restenosis, and vascular stenosis.

12. A method of claim 2, wherein the cholesterol-related disorder is selected from the group consisting of: elevated cholesterol, a condition associated with elevated cholesterol, a lipid disorder, hyperlipidemia, type I, type II, type III, type IV, and type V hyperlipidemia, secondary hypertriglyceridemia, hypercholesterolemia, xanthomatosis, and cholesterol acetyltransferase deficiency.

13. Method of treating MHF comprising administering to a subject in need thereof a pharmaceutically effective amount of a DKK1/4 antibody comprising the CDR1, CDR2 and CDR3 regions selected from Tables 5, 6, 7 and 8.

14. The method of claim 2 wherein the CDR1, CDR2 and CDR3 regions selected from Tables 5, 6, 7 and 8 are selected from SEQ ID NOs: 49-52 for a V_{H} CDR1, SEQ ID NOs: 53-63 for a V_{H} CDR2, SEQ ID NOs: 64-69 for a V_{H} CDR3, and SEQ ID NOs: 70-74 for a V_{L} CDR1, SEQ ID NOs: 75-79 for a V_{L} CDR2, SEQ ID NOs: 80-98 for a V_{L} CDR3.

15. The method of claim 2 wherein the CDR1, CDR2 and CDR3 regions selected from Tables 5, 6, 7 and 8 comprise a consensus sequence selected from SEQ ID NOs: 40-43 for a V_{H} CDR1, SEQ ID NOs: 44-47 for a V_{H} CDR2, SEQ ID NO: 48 for a V_{H} CDR3, and SEQ ID NOs: 113 and 116 for a V_{L} CDR1, SEQ ID NOs: 114 and 117 for a V_{L} CDR2, SEQ ID NOs: 115 and 118 for a V_{L} CDR3.


SEQUENCE LISTING (133 sequences)

SEQ ID NO: 1 - protein, 266 aa, Homo sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 2 - protein, 124 aa, Homo sapiens
QVQLVESGGGLVQPGGSLRLSCAASGFTFSNYGLHWVRQAPGKGLEWVSSISYYGSSTYYADSVKGRFTISRDNSKNTLYLQMNSLRAEDTAVYYCARDGSHMDKPPGYVFAFWGQGTLVTVSS

SEQ ID NO: 3 - protein, 114 aa, Homo sapiens
QVQLVESGGGLVQPGGSLRLSCAASGFTFSSYGMSWVRQAPGKGLEWVSGISGSGSYTYYADSVKGRFTISRDNSKNTLYLQMNSLRAEDTAVYYCARHYMDHWGQGTLVTVSS

SEQ ID NO: 4 - protein, 115 aa, Homo sapiens
QVQLVESGGGLVQPGGSLRLSCAASGFTFNNYGMTWVRQAPGKGLEWVSGISGSGSYTYYADSVKGRFTISRDNSKNTLYLQMNSLRAEDTAVYYCARTIYMDYWGQGTLVTVSS

SEQ ID NO: 5 - protein, 116 aa, Homo sapiens
QVQLVESGGGLVQPGGSLRLSCAASGFTFSSYWMSWVRQAPGKGLEWVSGISYSGSNTHYADSVKGRFTISRDNSKNTLYLQMNSLRAEDTAVYYCARMGIDLDYWGQGTLVTVSS

SEQ ID NO: 6 - protein, 116 aa, Homo sapiens
QVQLVESGGGLVQPGGSLRLSCAASGFTFSSYWMSWVRQAPGKGLEWVSVISSDSSSTYYADSVKGRFTISRDNSKNTLYLQMNSLRAEDTAVYYCARHGIDFDHWGQGTLVTVSS

SEQ ID NO: 7 - protein, 120 aa, Homo sapiens
QVQLVQSGAEVKKPGESLKISCKGSGYSFTNYYIGWVRQMPGKGLEWMGIIYPTDSYTNYSPSFQGQVTISADKSISTAYLQWSSLKASDTAMYYCARGIPFRMRGFDYWGQGTLVTVSS

SEQ ID NO: 8 - protein, 115 aa, Homo sapiens; identical to SEQ ID NO: 4

SEQ ID NO: 9 - protein, 115 aa, Homo sapiens; identical to SEQ ID NO: 4

SEQ ID NO: 10 - protein, 115 aa, Homo sapiens; identical to SEQ ID NO: 4

SEQ ID NO: 11 - protein, 116 aa, Homo sapiens; identical to SEQ ID NO: 5

SEQ ID NO: 12 - protein, 116 aa, Homo sapiens; identical to SEQ ID NO: 5

SEQ ID NO: 13 - protein, 116 aa, Homo sapiens; identical to SEQ ID NO: 5

SEQ ID NO: 14 - protein, 116 aa, Homo sapiens; identical to SEQ ID NO: 6

SEQ ID NO: 15 - protein, 118 aa, Homo sapiens
QVQLVESGGGLVQPGGSLRLSCAASGFTFSSYWMSWVRQAPGKGLEWVSSIEHKDAGYTTWYAAGVKGRFTISRDNSKNTLYLQMNSLRAEDTAVYYCARHGIDFDHWGQGTLVTVSS

SEQ ID NO: 16 - protein, 116 aa, Homo sapiens; identical to SEQ ID NO: 6

SEQ ID NO: 17 - protein, 116 aa, Homo sapiens; identical to SEQ ID NO: 6

SEQ ID NO: 18 - protein, 118 aa, Homo sapiens
QVQLVESGGGLVQPGGSLRLSCAASGFTFSSYWMSWVRQAPGKGLEWVSVIEHKDKGGTTYYAASVKGRFTISRDNSKNTLYLQMNSLRAEDTAVYYCARHGIDFDHWGQGTLVTVSS

SEQ ID NO: 19 - protein, 120 aa, Homo sapiens
QVQLVQSGAEVKKPGESLKISCKGSGYSFTNYYIGWVRQMPGKGLEWMGIIVPGTSYTIYSPSFQGQVTISADKSISTAYLQWSSLKASDTAMYYCARGIPFRMRGFDYWGQGTLVTVSS

SEQ ID NO: 20 - protein, 115 aa, Homo sapiens; identical to SEQ ID NO: 4

SEQ ID NO: 21 - protein, 109 aa, Homo sapiens
DIQMTQSPSSLSASVGDRVTITCRASQGIKNYLNWYQQKPGKAPKLLIGAASSLQSGVPSRFSGSGSGTDFTLTISSLQPEDFATYYCLQYYGMPPTFGQGTKVEIKRT

SEQ ID NO: 22 - protein, 109 aa, Homo sapiens
DIQMTQSPSSLSASVGDRVTITCRASQDISNYLHWYQQKPGKAPKLLIYGASNLQSGVPSRFSGSGSGTDFTLTISSLQPEDFAVYYCQQYDSIPMTFGQGTKVEIKRT

SEQ ID NO: 23 - protein, 110 aa, Homo sapiens
DIVLTQSPATLSLSPGERATLSCRASQNLFSPYLAWYQQKPGQAPRLLIYGASNRATGVPARFSGSGSGTDFTLTISSLEPEDFATYYCQQYGDEPITFGQGTKVEIKRT

SEQ ID NO: 24 - protein, 112 aa, Homo sapiens
DIALTQPASVSGSPGQSITISCTGTSSDVGGFNYVSWYQQHPGKAPKLMIHDGSNRPSGVSNRFSGSKSGNTASLTISGLQAEDEADYYCSTWDMTVDFVFGGGTKLTVLGQ

SEQ ID NO: 25 - protein, 113 aa, Homo sapiens
DIALTQPASVSGSPGQSITISCTGTSSDLGGYNYVSWYQQHPGKAPKLMIYDVNNRPSGVSNRFSGSKSGNTASLTISGLQAEDEADYYCQSYASGNTKVVFGGGTKLTVLGQ

SEQ ID NO: 26 - protein, 112 aa, Homo sapiens
DIVLTQPPSVSGAPGQRVTISCSGSSSNIGSSFVNWYQQLPGTAPKLLIGNNSNRPSGVPDRFSGSKSGTSASLAITGLQSEDEADYYCASFDMGSPNVVFGGGTKLTVLGQ

SEQ ID NO: 27 - protein, 110 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYLSLPTTFGQGTKVEIKRT

SEQ ID NO: 28 - protein, 110 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYMTLPLTFGQGTKVEINRT

SEQ ID NO: 29 - protein, 110 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYLTLPLTFGQGTKVEIKRT

SEQ ID NO: 30 - protein, 113 aa, Homo sapiens
DIALTQPASVSGSPGQSITISCTGTSSDVGGFNYVSWYQQHPGKAPKLMIHDGSNRPSGVSNRFSGSKSGNTASLTISGLQAEDEADYYCQSWDVSPITAVFGGGTKLTVLGQ

SEQ ID NO: 31 - protein, 113 aa, Homo sapiens
DIALTQPASVSGSPGQSITISCTGTSSDVGGFNYVSWYQQHPGKAPKLMIHDGSNRPSGVSNRFSGSKSGNTASLTISGLQAEDEADYYCQTWATSPLSSVFGGGTKLTVLGQ

SEQ ID NO: 32 - protein, 112 aa, Homo sapiens
DIALTQPASVSGSPGQSITISCTGTSSDVGGFNYVSWYQQHPGKAPKLMIHDGSNRPSGVSNRFSGSKSGNTASLTISGLQAEDEADYYCQTWDSLSFFVFGGGTKLTVLGQ

SEQ ID NO: 33 - protein, 113 aa, Homo sapiens
DIALTQPASVSGSPGQSITISCTGTSSDLGGYNYVSWYQQHPGKAPKLMIYDVNNRPSGVSNRFSGSKSGNTASLTISGLQAEDEADYYCQSYTYTPISPVFGGGTKLTVLGQ

SEQ ID NO: 34 - protein, 113 aa, Homo sapiens; identical to SEQ ID NO: 25

SEQ ID NO: 35 - protein, 113 aa, Homo sapiens
DIALTQPASVSGSPGQSITISCTGTSSDLGGYNYVSWYQQHPGKAPKLMIYDVNNRPSGVSNRFSGSKSGNTASLTISGLQAEDEADYYCQTYDQIKLSAVFGGGTKLTVLGQ

SEQ ID NO: 36 - protein, 113 aa, Homo sapiens
DIALTQPASVSGSPGQSITISCTGTSSDLGGYNYVSWYQQHPGKAPKLMIYDVNNRPSGVSNRFSGSKSGNTASLTISGLQAEDEADYYCQSYDSPTDSVVFGGGTKLTVLGQ

SEQ ID NO: 37 - protein, 113 aa, Homo sapiens; identical to SEQ ID NO: 25

SEQ ID NO: 38 - protein, 112 aa, Homo sapiens; identical to SEQ ID NO: 26

SEQ ID NO: 39 - protein, 110 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYMTLPLTFGQGTKVEIKRT

SEQ ID NO: 40 - protein, 10 aa, Artificial
  CDR consensus
GFTFNNYGMT

SEQ ID NO: 41 - protein, 10 aa, Artificial
  CDR consensus
GFTFSSYWMT

SEQ ID NO: 42 - protein, 10 aa, Artificial
  CDR consensus
GFTFXXYXXX

SEQ ID NO: 43 - protein, 10 aa, Artificial
  CDR consensus
GYSFTNYYIG

SEQ ID NO: 44 - protein, 17 aa, Artificial
  CDR consensus
GISGSGSYTYYADSVKG

SEQ ID NO: 45 - protein, 17 aa, Artificial
  CDR consensus
GISYSGSNTHYADSVKG

SEQ ID NO: 46 - protein, 17 aa, Artificial
  CDR consensus
VISSDSSSTYYADSVKG

SEQ ID NO: 47 - protein, 17 aa, Artificial
  CDR consensus
IIXPXXSYTXYSPSFQG

SEQ ID NO: 48 - protein, 7 aa, Artificial
  CDR consensus
XXIXXDX

SEQ ID NO: 49 - protein, 10 aa, ARTIFICIAL
  CDR fragment
GFTFSSYGMS

SEQ ID NO: 50 - protein, 10 aa, ARTIFICIAL
  CDR fragment
GFTFNNYGMT

SEQ ID NO: 51 - protein, 10 aa, ARTIFICIAL
  CDR fragment
GFTFSSYWMS

SEQ ID NO: 52 - protein, 10 aa, ARTIFICIAL
  CDR fragment
GYSFTNYYIG

SEQ ID NO: 53 - protein, 20 aa, ARTIFICIAL
  CDR fragment
WVSGISGSGSYTYYADSVKG

SEQ ID NO: 54 - protein, 20 aa, ARTIFICIAL
  CDR fragment
WVSGISERGVYIFYADSVKG

SEQ ID NO: 55 - protein, 20 aa, ARTIFICIAL
  CDR fragment
WVSGISYSGSNTHYADSVKG

SEQ ID NO: 56 - protein, 23 aa, ARTIFICIAL
  CDR fragment
WVSDIEHKRRAGGATSYAASVKG

SEQ ID NO: 57 - protein, 22 aa, ARTIFICIAL
  CDR fragment
WVSMIEHKTRGGTTDYAAPVKG

SEQ ID NO: 58 - protein, 20 aa, ARTIFICIAL
  CDR fragment
WVSVISSDSSSTYYADSVKG

SEQ ID NO: 59 - protein, 22 aa, ARTIFICIAL
  CDR fragment
WVSVIEHKSFGSATFYAASVKG

SEQ ID NO: 60 - protein, 22 aa, ARTIFICIAL
  CDR fragment
WVSVIEHKDKGGTTYYAASVKG

SEQ ID NO: 61 - protein, 22 aa, ARTIFICIAL
  CDR fragment
WVSSIEHKDAGYTTWYAAGVKG

SEQ ID NO: 62 - protein, 20 aa, ARTIFICIAL
  CDR fragment
WMGIIYPTDSYTNYSPSFQG

SEQ ID NO: 63 - protein, 20 aa, ARTIFICIAL
  CDR fragment
WMGIIYPGTSYTIYSPSFGQ

SEQ ID NO: 64 - protein, 5 aa, ARTIFICIAL
  CDR fragment
HYMDH

SEQ ID NO: 65 - protein, 6 aa, ARTIFICIAL
  CDR fragment
TIYMDY

SEQ ID NO: 66 - protein, 7 aa, ARTIFICIAL
  CDR fragment
MGIDLDY

SEQ ID NO: 67 - protein, 7 aa, ARTIFICIAL
  CDR fragment
HGIDFDH

SEQ ID NO: 68 - protein, 11 aa, ARTIFICIAL
  CDR fragment
GIPFRMRGFDY

SEQ ID NO: 69 - protein, 15 aa, ARTIFICIAL
  CDR fragment
DGSHMDKPPGYVFAF

SEQ ID NO: 70 - protein, 11 aa, ARTIFICIAL
  CDR fragment
RASQDISNYLH

SEQ ID NO: 71 - protein, 12 aa, ARTIFICIAL
  CDR fragment
RASQNLFSPYLA

SEQ ID NO: 72 - protein, 14 aa, ARTIFICIAL
  CDR fragment
TGTSSDVGGFNYVS

SEQ ID NO: 73 - protein, 14 aa, ARTIFICIAL
  CDR fragment
TGTSSDLGGYNYVS

SEQ ID NO: 74 - protein, 13 aa, ARTIFICIAL
  CDR fragment
SGSSSNIGSSFVN

SEQ ID NO: 75 - protein, 11 aa, ARTIFICIAL
  CDR fragment
LLIYGASNLQS

SEQ ID NO: 76 - protein, 11 aa, ARTIFICIAL
  CDR fragment
LLIYGASNRAT

SEQ ID NO: 77 - protein, 11 aa, ARTIFICIAL
  CDR fragment
LMIHDGSNRPS

SEQ ID NO: 78 - protein, 11 aa, ARTIFICIAL
  CDR fragment
LMIYDVNNRPS

SEQ ID NO: 79 - protein, 11 aa, ARTIFICIAL
  CDR fragment
LLIGNNSNRPS

SEQ ID NO: 80 - protein, 8 aa, ARTIFICIAL
  CDR fragment
LQYYGMPP

SEQ ID NO: 81 - protein, 8 aa, ARTIFICIAL
  CDR fragment
QQYDSIPM

SEQ ID NO: 82 - protein, 8 aa, ARTIFICIAL
  CDR fragment
QQYGDEPI

SEQ ID NO: 83 - protein, 8 aa, ARTIFICIAL
  CDR fragment
QQYLSLPT

SEQ ID NO: 84 - protein, 8 aa, ARTIFICIAL
  CDR fragment
QQYLTLPL

SEQ ID NO: 85 - protein, 7 aa, ARTIFICIAL
  CDR fragment
QQYLFPL

SEQ ID NO: 86 - protein, 8 aa, ARTIFICIAL
  CDR fragment
QQYMTLPL

SEQ ID NO: 87 - protein, 9 aa, ARTIFICIAL
  CDR fragment
STWDMTVDF

SEQ ID NO: 88 - protein, 10 aa, ARTIFICIAL
  CDR fragment
QSWDVSPITA

SEQ ID NO: 89 - protein, 9 aa, ARTIFICIAL
  CDR fragment
QTWDSLSFF

SEQ ID NO: 90 - protein, 10 aa, ARTIFICIAL
  CDR fragment
QSWGVGPGGF

SEQ ID NO: 91 - protein, 10 aa, ARTIFICIAL
  CDR fragment
QTWATSPLSS

SEQ ID NO: 92 - protein, 10 aa, ARTIFICIAL
  CDR fragment
QSYASGNTKV

SEQ ID NO: 93 - protein, 10 aa, ARTIFICIAL
  CDR fragment
QSYTYTPISP

SEQ ID NO: 94 - protein, 10 aa, ARTIFICIAL
  CDR fragment
QTYDQIKLSA

SEQ ID NO: 95 - protein, 10 aa, ARTIFICIAL
  CDR fragment
QSYDPFLDVV

SEQ ID NO: 96 - protein, 10 aa, Artificial
  CDR fragment
QSYDSPTDSV

SEQ ID NO: 97 - protein, 10 aa, ARTIFICIAL
  CDR fragment
QSYASGNTKV

SEQ ID NO: 98 - protein, 10 aa, ARTIFICIAL
  CDR fragment
ASFDMGSPNV

SEQ ID NO: 99 - DNA, 654 bp, Homo sapiens
gatatcgcactgacccagccagcttcagtgagcggctcaccaggtcagagcattaccatctcgtgtacgggtactagcagcgatgttggtggttttaattatgtgtcttggtaccagcagcatcccgggaaggcgccgaaacttatgattcatgatggttctaatcgtccctcaggcgtgagcaaccgttttagcggatccaaaagcggcaacaccgcgagcctgaccattagcggcctgcaagcggaagacgaagcggattattattgccagtcttgggatgtttctcctattactgctgtgtttggcggcggcacgaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 100 - protein, 217 aa, Homo sapiens
DIALTQPASVSGSPGQSITISCTGTSSDVGGFNYVSWYQQHPGKAPKLMIHDGSNRPSGVSNRFSGSKSGNTASLTISGLQAEDEADYYCQSWDVSPITAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - DNA, 654 bp, Homo sapiens
gatatcgcactgacccagccagcttcagtgagcggctcaccaggtcagagcattaccatctcgtgtacgggtactagcagcgatcttggtggttataattatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgccagacttatgatcagattaagttgtctgctgtgtttggcggcggcacgaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 102 - protein, 217 aa, Homo sapiens
DIALTQPASVSGSPGQSITISCTGTSSDLGGYNYVSWYQQHPGKAPKLMIYDVNNRPSGVSNRFSGSKSGNTASLTISGLQAEDEADYYCQTYDQIKLSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - DNA, 648 bp, Homo sapiens
gatatcgtgctgacccagagcccggcgaccctgagcctgtctccgggcgaacgtgcgaccctgagctgcagagcgagccagaatcttttttctccttatctggcttggtaccagcagaaaccaggtcaagcaccgcgtctattaatttatggtgcttctaatcgtgcaactggggtcccggcgcgttttagcggctctggatccggcacggattttaccctgaccattagcagcctggaacctgaagactttgcggtgtattattgccagcagtatcttactcttcctcttacctttggccagggtacgaaagtcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 104 - protein, 215 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYLTLPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 648 bp, Homo sapiens
gatatcgtgctgacccagagcccggcgaccctgagcctgtctccgggcgaacgtgcgaccctgagctgcagagcgagccagaatcttttttctccttatctggcttggtaccagcagaaaccaggtcaagcaccgcgtctattaatttatggtgcttctaatcgtgcaactggggtcccggcgcgttttagcggctctggatccggcacggattttaccctgaccattagcagcctggaacctgaagactttgcggtgtattattgccagcagtatatgactcttcctcttacctttggccagggtacgaaagtcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 106 - protein, 215 aa, Homo sapiens
DIVLTQSPATLSLSPGERATLSCRASQNLFSPYLAWYQQKPGQAPRLLIYGASNRATGVPARFSGSGSGTDFTLTISSLEPEDFAVYYCQQYMTLPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - DNA, 1347 bp, Homo sapiens
caggcacaggtgcaattggtggaaagcggcggcggcctggtgcaaccgggcggcagcctgcgtctgagctgcgcggcctccggatttaccttttcttcttattggatgtcttgggtgcgccaagcccctgggaagggtctcgagtgggtgagcggtatctcttattctggtagcaatacccattatgcggatagcgtgaaaggccgttttaccatttcacgtgataattcgaaaaacaccctgtatctgcaaatgaacagcctgcgtgcggaagatacggccgtgtattattgcgcgcgtatgggtattgatcttgattattggggccaaggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 108 - protein, 446 aa, Homo sapiens
QVQLVESGGGLVQPGGSLRLSCAASGFTFSSYWMSWVRQAPGKGLEWVSGISYSGSNTHYADSVKGRFTISRDNSKNTLYLQMNSLRAEDTAVYYCARMGIDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1347 bp, Homo sapiens
caggcacaggtgcaattggtggaaagcggcggcggcctggtgcaaccgggcggcagcctgcgtctgagctgcgcggcctccggatttaccttttcttcttattggatgtcttgggtgcgccaagcccctgggaagggtctcgagtgggtgagcgttatctcttctgattctagctctacctattatgcggatagcgtgaaaggccgttttaccatttcacgtgataattcgaaaaacaccctgtatctgcaaatgaacagcctgcgtgcggaagatacggccgtgtattattgcgcgcgtcatggtattgattttgatcattggggccaaggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 110 - protein, 446 aa, Homo sapiens
QVQLVESGGGLVQPGGSLRLSCAASGFTFSSYWMSWVRQAPGKGLEWVSVISSDSSSTYYADSVKGRFTISRDNSKNTLYLQMNSLRAEDTAVYYCARHGIDFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1338 bp, Homo sapiens
caggtgcagctggtggagagcggcggaggactggtgcagcctggcggcagcctgagactgagctgtgccgccagcggcttcaccttcaacaactacggcatgacctgggtgaggcaggcccctggcaagggcctggagtgggtgtccggcatcagcggcagcggcagctacacctactacgccgacagcgtgaagggcaggttcaccatcagccgggacaacagcaagaacaccctgtacctgcagatgaacagcctgagagccgaggacaccgccgtgtactactgtgcccggaccatctacatggactactggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 112 - protein, 445 aa, Homo sapiens
QVQLVESGGGLVQPGGSLRLSCAASGFTFNNYGMTWVRQAPGKGLEWVSGISGSGSYTYYADSVKGRFTISRDNSKNTLYLQMNSLRAEDTAVYYCARTIYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 11 aa, Artificial
  Consensus CDR
RASQXXXXXYX

SEQ ID NO: 114 - protein, 11 aa, Artificial
  Consensus CDR
LLIYGASNXXX

SEQ ID NO: 115 - protein, 8 aa, Artificial
  Consensus CDR
QQYXXXPX

SEQ ID NO: 116 - protein, 14 aa, Artificial
  Consensus CDR
TGTSSDVGGFNYVS

SEQ ID NO: 117 - protein, 11 aa, Artificial
  Consensus CDR
LMIXDXXNRPS

SEQ ID NO: 118 - protein, 9 aa, Artificial
  Consensus CDR
XXWDXXXXX

SEQ ID NO: 119 - DNA, 333 bp, Homo sapiens
acgcgttgcgatatcgccctgacccagcccgccagcgtgtccggcagccctggccagagcatcaccatcagctgtaccggcaccagcagcgacctgggcggctacaactacgtgtcctggtatcagcagcaccccggcaaggcccccaagctgatgatctacgacgtgaacaacagacctagcggcgtgtccaacagattcagcggcagcaagagcggcaacaccgccagcctgaccatctctggcctgcaggctgaggacgaggccgactactactgccagacctacgaccagatcaagctgtccgccgtgtttggcggcggaacaaagctt

SEQ ID NO: 120 - DNA, 333 bp, Homo sapiens
aagctttgttccgccgccaaacacggcggacagcttgatctggtcgtaggtctggcagtagtagtcggcctcgtcctcagcctgcaggccagagatggtcaggctggcggtgttgccgctcttgctgccgctgaatctgttggacacgccgctaggtctgttgttcacgtcgtagatcatcagcttgggggccttgccggggtgctgctgataccaggacacgtagttgtagccgcccaggtcgctgctggtgccggtacagctgatggtgatgctctggccagggctgccggacacgctggcgggctgggtcagggcgatatcgcaacgcgt

SEQ ID NO: 121 - protein, 111 aa, Homo sapiens
TRCDIALTQPASVSGSPGQSITISCTGTSSDLGGYNYVSWYQQHPGKAPKLMIYDVNNRPSGVSNRFSGSKSGNTASLTISGLQAEDEADYYCQTYDQIKLSAVFGGGTKL

SEQ ID NO: 122 - DNA, 361 bp, Homo sapiens
gagtccattgggagtgcaggcccaggtgcagctggtggagagcggcggaggactggtgcagcctggcggcagcctgagactgagctgtgccgccagcggcttcaccttcagcagctactggatgagctgggtgaggcaggcccctggcaagggcctggagtgggtgtccgtgatcagcagcgatagcagcagcacctactacgccgatagcgtgaagggccggttcaccatcagccgggacaacagcaagaacaccctgtacctgcagatgaacagcctgagagccgaggacaccgccgtgtactactgtgccaggcacggcatcgacttcgaccactggggccagggcaccctggtcacc

SEQ ID NO: 123 - DNA, 361 bp, Homo sapiens
ggtgaccagggtgccctggccccagtggtcgaagtcgatgccgtgcctggcacagtagtacacggcggtgtcctcggctctcaggctgttcatctgcaggtacagggtgttcttgctgttgtcccggctgatggtgaaccggcccttcacgctatcggcgtagtaggtgctgctgctatcgctgctgatcacggacacccactccaggcccttgccaggggcctgcctcacccagctcatccagtagctgctgaaggtgaagccgctggcggcacagctcagtctcaggctgccgccaggctgcaccagtcctccgccgctctccaccagctgcacctgggcctgcactcccaatggactc

SEQ ID NO: 124 - protein, 117 aa, Homo sapiens
GVQAQVQLVESGGGLVQPGGSLRLSCAASGFTFSSYWMSWVRQAPGKGLEWVSVISSDSSSTYYADSVKGRFTISRDNSKNTLYLQMNSLRAEDTAVYYCARHGIDFDHWGQGTLVT

SEQ ID NO: 125 - protein, 127 aa, Homo sapiens
  MISC_FEATURE (99)..(116): Wherein X may be any amino acid
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARXXXXXXXXXXXXXXXXXXWGQGTLVTVSS

SEQ ID NO: 126 - protein, 127 aa, Homo sapiens
  MISC_FEATURE (99)..(116): Wherein X may be any amino acid
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARXXXXXXXXXXXXXXXXXXWGQGTLVTVSS

SEQ ID NO: 127 - protein, 110 aa, Homo sapiens
  MISC_FEATURE (90)..(97): Wherein X may be any amino acid
DIQMTQSPSSLSASVGDRVTITCRASQGISSYLAWYQQKPGKAPKLLIYAASSLQSGVPSRFSGSGSGTDFTLTISSLQPEDFATVYYCXXXXXXXXTFGQGTKVEIKRT

SEQ ID NO: 128 - protein, 111 aa, Homo sapiens
  MISC_FEATURE (91)..(98): Wherein X may be any amino acid
DIVLTQSPATLSLSPGERATLSCRASQSVSSSYLAWYQQKPGQAPRLLIYGASSRATGVPARFSGSGSGTDFTLTISSLEPEDFATVYYCXXXXXXXXTFGQGTKVEIKRT

SEQ ID NO: 129 - protein, 113 aa, Homo sapiens
  MISC_FEATURE (91)..(100): Wherein X may be any amino acid
DIALTQPASVSGSPGQSITISCTGTSSDVGGYNYVSWYQQHPGKAPKLMIYDVSNRPSGVSNRFSGSKSGNTASLTISGLQAEDEADYYCXXXXXXXXXXVFGGGTKLTVLGQ

SEQ ID NO: 130 - protein, 112 aa, Homo sapiens
  MISC_FEATURE (90)..(99): Wherein X may be any amino acid
DIVLTQPPSVSGAPGQRVTISCSGSSSNIGSNYVSWYQQLPGTAPKLLIYDNNQRPSGVPDRFSGSKSGTSASLAITGLQSEDEADYYCXXXXXXXXXXVFGGGTKLTVLGQ

SEQ ID NO: 131 - protein, 259 aa, Homo sapiens
MAALMRSKDSSCCLLLLAAVLMVESSQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 132 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALGEPAAAAAALLGGEEI

SEQ ID NO: 133 - protein, 224 aa, Homo sapiens
MVAAVLLGLSWLCSPLGALVLDFNNIRSSADLHGARKGSQCLSDTDCNTRKFCLQPRDEKPFCATCRGLRRRCQRDAMCCPGTLCVNDVCTTMEDATPILERQLDEQDGTHAEGTTGHPVQENQPKRKPSIKKSQGRKGQEGESCLRTFDCGPGLCCARHFWTKICKPVLLEGQVCSRRGHKDTAQAPEIFQRCDCGPGLLCRSQLTSNRQHARLRVCQKIEKL